(19) United States
(12) Patent Application Publication
(10) Pub. No.: US 2011/0009335 A1
(43) Pub. Date: Jan. 13, 2011

(54) Anticancer Treatments

(75) Inventors: Doreen LePage, Cambridge, MA (US); Pablo Manuel Aviles Marin, Madrid (ES); Maria Jose Guillen, Madrid (ES)

Correspondence Address: KING & SPALDING, 1185 AVENUE OF THE AMERICAS, NEW YORK, NY, 10036-4003, US

(73) Assignee: Pharma Mar, S.A., Madrid (ES)

(21) Appl. No.: 12/920,427

(22) PCT Filed: Mar. 6, 2009
(86) PCT No.: PCT/US09/36327

§ 371 Date: Aug. 31, 2010

Related U.S. Application Data
(60) Provisional application No. 61/034,870, filed on Mar. 7, 2008.

(51) Int. Cl. A61K 38/15; A61P 35/00

(52) U.S. Cl. 514/19.3

(57) ABSTRACT

The present invention relates to combinations of aplidine with another anticancer drug selected from sorafenib, temsirolimus, and sunitinib, and the use of these combinations in the treatment of cancer.

### FIELD OF THE INVENTION

The present invention relates to the combination of aplidine with other anticancer drugs, in particular other anticancer drugs selected from sorafenib, sunitinib, and temsirolimus, and the use of these combinations in the treatment of cancer.

### BACKGROUND OF THE INVENTION

Cancer develops when cells in a part of the body begin to grow out of control. Although there are many kinds of cancer, they all arise from out-of-control growth of abnormal cells. Cancer cells can invade nearby tissues and can spread through the bloodstream and lymphatic system to other parts of the body. There are several main types of cancer. Carcinoma is a malignant neoplasm, which is an uncontrolled and progressive abnormal growth, arising from epithelial cells. Epithelial cells cover internal and external surfaces of the body, including organs, lining of vessels and other small cavities. Sarcoma is cancer arising from cells in bone, cartilage, fat, muscle, blood vessels, or other connective or supportive tissue. Leukemia is cancer that arises in blood-forming tissue such as the bone marrow, and causes large numbers of abnormal blood cells to be produced and enter the bloodstream. Lymphoma and multiple myeloma are cancers that arise from cells of the immune system.

In addition, cancer is invasive and tends to infiltrate the surrounding tissues and give rise to metastases. It can spread directly into surrounding tissues and also may be spread through the lymphatic and circulatory systems to other parts of the body.

Many treatments are available for cancer, including surgery and radiation for localised disease, and chemotherapy. However, the efficacy of available treatments for many cancer types is limited, and new, improved forms of treatment showing clinical benefits are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells, and would also retain its efficacy against cancer cells, even after prolonged exposure to the drug. Unfortunately, none of the current chemotherapies with known agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

Aplidine (dehydrodidemnin B) is a cyclic depsipeptide that was isolated from a Mediterranean marine tunicate, Aplidium albicans, and is the subject of WO 91/04985. It is related to compounds known as didemnins, and has the following structure:

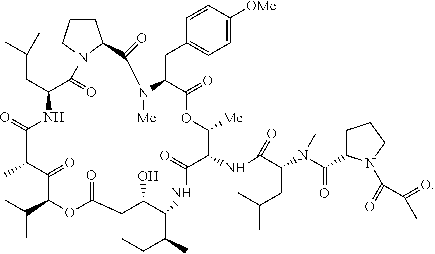

More information on aplidine, its uses, formulations and synthesis can be found in patent applications WO 91/04985, WO 99/42125, WO 01/35974, WO 01/76616, WO 2004/084812, WO 02/30441, WO 02/02596, WO 03/33013, WO 2004/080477, WO 2004/080421, WO 2007/101235, WO 2008/135793, and PCT/EP2008/064117. We incorporate by specific reference the content of each of these patent application texts.

In both animal preclinical studies and human clinical Phase I studies, aplidine has been shown to have cytotoxic potential against a broad spectrum of tumor types including leukemia and lymphoma. See for example:
Faircloth, G. et al.: “Dehydrodidemnin B (DDB) a new marine derived anticancer agent with activity against experimental tumour models”, 9th NCI-EORTC Symp. New Drugs Cancer Ther. (March 12-15, Amsterdam) 1996, Abst 111;
Faircloth, G. et al.: “Preclinical characterization of aplidine, a new marine anticancer depsipeptide”, Proc. Amer. Assoc. Cancer Res. 1997, 38: Abst 692;
Depenbrock H, Peter R, Faircloth G T, Manzanares I, Jimeno J, Hanauske A R.: “In vitro activity of aplidine, a new marine-derived anti-cancer compound, on freshly explanted clonogenic human tumour cells and haematopoietic precursor cells” Br. J. Cancer, 1998; 78: 739-744;
Faircloth G, Grant W, Nam S, Jimeno J, Manzanares I, Rinehart K.: “Schedule-dependency of aplidine, a marine depsipeptide with antitumor activity”, Proc. Am. Assoc. Cancer Res. 1999; 40: 394;
Broggini M, Marchini S, D'Incalci M, Taraboletti G, Giavazzi R, Faircloth G, Jimeno J.: “Aplidine blocks VEGF secretion and VEGF/VEGF-R1 autocrine loop in a human leukemic cell line”, Clin. Cancer Res. 2000; 6 (suppl): 4509;
Erba E, Bassano L, Di Liberti G, Muradore I, Chiorino G, Ubezio P, Vignati S, Codegoni A, Desiderio M A, Faircloth G, Jimeno J and D'Incalci M.: “Cell cycle phase perturbations and apoptosis in tumour cells induced by aplidine”, Br. J. Cancer 2002; 86: 1510-1517;
Paz-Ares L, Anthony A, Pronk L, Twelves C, Alonso S, Cortes-Funes H, Celli N, Gomez C, Lopez-Lazaro L, Guzman C, Jimeno J, Kaye S.: “Phase I clinical and pharmacokinetic study of aplidine, a new marine didemnin, administered as 24-hour infusion weekly” Clin. Cancer Res. 2000; 6 (suppl): 4509;
Raymond E, Ady-Vago N, Baudin E, Ribrag V, Faivre S, Lecot F, Wright T, Lopez Lazaro L, Guzman C, Jimeno J, Ducreux M, Le Chevalier T, Armand J P.: “A phase I and pharmacokinetic study of aplidine given as a 24-hour continuous infusion every other week in patients with solid tumor and lymphoma”, Clin. Cancer Res. 2000; 6 (suppl): 4510;
Maroun J, Belanger K, Seymour L, Soulieres D, Charpentier D, Goel R, Stewart D, Tomiak E, Jimeno J, Matthews S.: “Phase I study of aplidine in a 5 day bolus q 3 weeks in patients with solid tumors and lymphomas”, Clin. Cancer Res. 2000; 6 (suppl): 4509;
Izquierdo M A, Bowman A, Martinez M, Cicchella B, Jimeno J, Guzman C, Germa J, Smyth J.: “Phase I trial of aplidine given as a 1 hour intravenous weekly infusion in patients with advanced solid tumors and lymphoma”, Clin. Cancer Res. 2000; 6 (suppl): 4509.

Mechanistic studies indicate that aplidine can block VEGF secretion in ALL-MOLT4 cells, and in vitro cytotoxic activity at low concentrations (5 nM) has been observed in AML and ALL samples from pediatric patients with de novo or relapsed ALL and AML. Aplidine appears to induce both a G1 and a G2 arrest in drug treated leukemia cells in vitro. Apart from down regulation of the VEGF receptor, little else is known about the mode(s) of action of aplidine.

In phase I clinical studies with aplidine, L-carnitine was given as a 24 hour pretreatment or co-administered to prevent myelotoxicity, see for example WO 02/30441. Co-administration of L-carnitine is thought to improve the recovery from drug-induced muscular toxicity.

Previously, in vitro and in vivo assays were conducted with aplidine in combination with other anticancer agents in order to evaluate whether the assayed drug combinations were useful in combination therapy for the treatment of leukemia and lymphoma. Specifically, in WO 2004/080421, aplidine was evaluated in combination with methotrexate, cytosine arabinoside, mitoxantrone, vinblastine, methylprednisolone, and doxorubicin for the treatment of leukemia and lymphoma. On the other hand, in WO 2007/101235, aplidine was specifically evaluated in combination with paclitaxel, doxorubicin, cisplatin, arsenic trioxide, 5-fluorouracil, cytosine arabinoside, carboplatin, 7-ethyl-10-hydroxycamptothecin, etoposide, melphalan, dexamethasone, cyclophosphamide, bortezomib, erlotinib, trastuzumab, lenalidomide, interleukin-2, interferon-α 2, dacarbazine, bevacizumab, idarubicin, thalidomide, and rituximab for the treatment of lung cancer, breast cancer, colon cancer, prostate cancer, renal cancer, melanoma, multiple myeloma, leukemia and lymphoma. In WO 2008/135793, aplidine was specifically evaluated in combination with carboplatin providing a schedule and dosage feasible for the administration of the combination of both drugs in human patients. Finally, in PCT/EP2008/064117, aplidine was specifically evaluated in combination with gemcitabine for the treatment of pancreatic carcinoma.

Since cancer is a leading cause of death in animals and humans, several efforts have been and are still being undertaken in order to obtain a safe and effective therapy to be administered to patients suffering from a cancer. The problem to be solved by the present invention is to provide anticancer therapies that are useful in the treatment of cancer.

### SUMMARY OF THE INVENTION

The present invention establishes that aplidine potentiates other anticancer agents, in particular sorafenib, sunitinib, and temsirolimus, and therefore aplidine and other anticancer agents can be successfully used in combination therapy for the treatment of cancer. Thus, this invention is directed to pharmaceutical compositions, kits, methods for the treatment of cancer using combination therapies, and uses of aplidine in the manufacture of a medicament for combination therapy.

In accordance with one aspect of this invention, we provide effective combination therapies for the treatment of cancer based on aplidine and using another anticancer drug selected from sorafenib, sunitinib, and temsirolimus.

In another embodiment, the invention encompasses a method of treating cancer comprising administering to a patient in need of such treatment a therapeutically effective amount of aplidine, or a pharmaceutically acceptable salt thereof, and a therapeutically effective amount of another anticancer drug selected from sorafenib, sunitinib, and temsirolimus, or a pharmaceutically acceptable salt thereof, administered prior, during, or after administering aplidine. The two drugs may form part of the same composition, or be provided as a separate composition for administration at the same time or at a different time.

In another aspect, the invention encompasses a method of potentiating the therapeutic efficacy of an anticancer drug selected from sorafenib, sunitinib, and temsirolimus in the treatment of cancer, which comprises administering to a patient in need thereof a therapeutically effective amount of aplidine, or a pharmaceutically acceptable salt thereof. Aplidine is administered prior, during, or after administering sorafenib, sunitinib, or temsirolimus.

In another embodiment, the invention encompasses the use of aplidine, or a pharmaceutically acceptable salt thereof, for the manufacture of a medicament for the treatment of cancer, in combination therapy with another anticancer drug selected from sorafenib, sunitinib, and temsirolimus.

In a related embodiment, the invention encompasses the use of an anticancer drug selected from sorafenib, sunitinib, and temsirolimus, or a pharmaceutically acceptable salt thereof, for the manufacture of a medicament for the treatment of cancer, in combination therapy with aplidine.

In a further aspect, the invention encompasses a pharmaceutical composition comprising aplidine, or a pharmaceutically acceptable salt thereof, and/or another anticancer drug selected from sorafenib, sunitinib, and temsirolimus, or a pharmaceutically acceptable salt thereof, to be used in combination therapy for the treatment of cancer.

The invention also encompasses a kit for use in the treatment of cancer which comprises a dosage form of aplidine, or a pharmaceutically acceptable salt thereof, and/or a dosage form of another anticancer drug selected from sorafenib, sunitinib, and temsirolimus, or a pharmaceutically acceptable salt thereof, and instructions for the use of both drugs in combination.

In one preferred aspect, the present invention is concerned with synergistic combinations of aplidine or a pharmaceutically acceptable salt thereof, with another anticancer drug selected from sorafenib, sunitinib, and temsirolimus, or a pharmaceutically acceptable salt thereof.

### BRIEF DESCRIPTION OF THE FIGURES

FIG. 1. Tumor weight evolution (mean±SEM) of CAKI-1 tumors in mice treated with Control (vehicle), aplidine (APLIDIN®), sorafenib or aplidine plus sorafenib. Aplidine was administered at a dose of 0.06 mg/kg/day and sorafenib at a dose of 60 mg/kg/day. Treatments started on day 13 post-implantation.

FIG. 2. Tumor weight evolution (mean±SEM) of CAKI-1 tumors in mice treated with Control (vehicle), aplidine (APLIDIN®), sorafenib or aplidine plus sorafenib. Aplidine was administered at a dose of 0.06 mg/kg/day and sorafenib at a dose of 30 mg/kg/day. Treatments started on day 13 post-implantation.

FIG. 3. Tumor weight evolution (mean±SEM) of CAKI-1 tumors in mice treated with Control (vehicle), aplidine (APLIDIN®), sorafenib or aplidine plus sorafenib. Aplidine was administered at a dose of 0.04 mg/kg/day and sorafenib at a dose of 60 mg/kg/day. Treatments started on day 13 post-implantation.

FIG. 4. Tumor weight evolution (mean±SEM) of CAKI-1 tumors in mice treated with Control (vehicle), aplidine (APLIDIN®), sorafenib or aplidine plus sorafenib. Aplidine was administered at a dose of 0.04 mg/kg/day and sorafenib at a dose of 30 mg/kg/day. Treatments started on day 13 post-implantation.

FIG. 5. Tumor weight evolution (mean±SEM) of MRI-H-121 tumors in mice treated with Control (vehicle), aplidine (APLIDIN®), sorafenib or aplidine plus sorafenib. Aplidine was administered at a dose of 0.06 mg/kg/day and sorafenib at a dose of 60 mg/kg/day. Treatments started on day 14 post-implantation.

FIG. 6. Tumor weight evolution (mean±SEM) of MRI-H-121 tumors in mice treated with Control (vehicle), aplidine (APLIDIN®), sorafenib or aplidine plus sorafenib. Aplidine was administered at a dose of 0.06 mg/kg/day and sorafenib at a dose of 30 mg/kg/day. Treatments started on day 14 post-implantation.

FIG. 7. Tumor weight evolution (mean±SEM) of MRI-H-121 tumors in mice treated with Control (vehicle), aplidine (APLIDIN®), sorafenib or aplidine plus sorafenib. Aplidine was administered at a dose of 0.04 mg/kg/day and sorafenib at a dose of 60 mg/kg/day. Treatments started on day 14 post-implantation.

FIG. 8. Tumor weight evolution (mean±SEM) of MRI-H-121 tumors in mice treated with Control (vehicle), aplidine (APLIDIN®), sorafenib or aplidine plus sorafenib. Aplidine was administered at a dose of 0.04 mg/kg/day and sorafenib at a dose of 30 mg/kg/day. Treatments started on day 14 post-implantation.

FIG. 9. Tumor weight evolution (mean±SEM) of A498 tumors in mice treated with Control (vehicle), aplidine (APLIDIN®), sorafenib or aplidine plus sorafenib. Aplidine was administered at a dose of 0.06 mg/kg/day and sorafenib at a dose of 60 mg/kg/day. Treatments started on day 26 post-implantation.

FIG. 10. Tumor weight evolution (mean±SEM) of A498 tumors in mice treated with Control (vehicle), aplidine (APLIDIN®), sorafenib or aplidine plus sorafenib. Aplidine was administered at a dose of 0.06 mg/kg/day and sorafenib at a dose of 30 mg/kg/day. Treatments started on day 26 post-implantation.

FIG. 11. Tumor weight evolution (mean±SEM) of A498 tumors in mice treated with Control (vehicle), aplidine (APLIDIN®), sorafenib or aplidine plus sorafenib. Aplidine was administered at a dose of 0.04 mg/kg/day and sorafenib at a dose of 60 mg/kg/day. Treatments started on day 26 post-implantation.

FIG. 12. Tumor weight evolution (mean±SEM) of A498 tumors in mice treated with Control (vehicle), aplidine (APLIDIN®), sorafenib or aplidine plus sorafenib. Aplidine was administered at a dose of 0.04 mg/kg/day and sorafenib at a dose of 30 mg/kg/day. Treatments started on day 26 post-implantation.

FIG. 13. Tumor weight evolution (mean±SEM) of CAKI-1 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 40 mg/kg/day. Treatments started on day 23 post-implantation.

FIG. 14. Tumor weight evolution (mean±SEM) of CAKI-1 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 30 mg/kg/day. Treatments started on day 23 post-implantation.

FIG. 15. Tumor weight evolution (mean±SEM) of CAKI-1 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 40 mg/kg/day. Treatments started on day 23 post-implantation.

FIG. 16. Tumor weight evolution (mean±SEM) of CAKI-1 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 30 mg/kg/day. Treatments started on day 23 post-implantation.

FIG. 17. Tumor weight evolution (mean±SEM) of MRI-H-121 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 40 mg/kg/day. Treatments started on day 10 post-implantation.

FIG. 18. Tumor weight evolution (mean±SEM) of MRI-H-121 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 30 mg/kg/day. Treatments started on day 10 post-implantation.

FIG. 19. Tumor weight evolution (mean±SEM) of MRI-H-121 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 40 mg/kg/day. Treatments started on day 10 post-implantation.

FIG. 20. Tumor weight evolution (mean±SEM) of MRI-H-121 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 30 mg/kg/day. Treatments started on day 10 post-implantation.

FIG. 21. Tumor weight evolution (mean±SEM) of A498 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 40 mg/kg/day. Treatments started on day 19 post-implantation.

FIG. 22. Tumor weight evolution (mean±SEM) of A498 tumors in mice treated with Control (vehicle), aplidine (APLIDIN®), sunitinib (SUTENT®) or aplidine plus sunitinib. Aplidine was administered at a dose of 0.06 mg/kg/day and sunitinib at a dose of 30 mg/kg/day. Treatments started on day 19 post-implantation.

FIG. 23. Tumor weight evolution (mean±SEM) of A498 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 40 mg/kg/day. Treatments started on day 19 post-implantation.

FIG. 24. Tumor weight evolution (mean±SEM) of A498 tumors in mice treated with Control (vehicle), aplidine (APLIDIN®), sunitinib (SUTENT®) or aplidine plus sunitinib. Aplidine was administered at a dose of 0.04 mg/kg/day and sunitinib at a dose of 30 mg/kg/day. Treatments started on day 19 post-implantation.

FIG. 25. Tumor volume evolution (mean±SEM) of MRI-H-121 tumors in mice treated with Control (vehicle), aplidine (APLIDIN®), temsirolimus (TORISEL®) or aplidine plus temsirolimus. Aplidine was administered at a dose of 0.06 mg/kg/day and temsirolimus at a dose of 20 mg/kg/day. Treatments started on day 12 post-implantation.

FIG. 26. Tumor volume evolution (mean±SEM) of MRI-H-121 tumors in mice treated with Control (vehicle), aplidine (APLIDIN®), temsirolimus (TORISEL®) or aplidine plus temsirolimus. Aplidine was administered at a dose of 0.06 mg/kg/day and temsirolimus at a dose of 10 mg/kg/day. Treatments started on day 12 post-implantation.

FIG. 27. Tumor weight evolution (mean±SEM) of CAKI-1 tumors in mice treated with Control (vehicle), aplidine, temsirolimus or aplidine plus temsirolimus. Aplidine was administered at a dose of 0.06 mg/kg/day and temsirolimus at a dose of 10 mg/kg/day. Treatments started on day 21 post-implantation.

FIG. 28. Tumor weight evolution (mean±SEM) of CAKI-1 tumors in mice treated with Control (vehicle), aplidine, temsirolimus or aplidine plus temsirolimus. Aplidine was administered at a dose of 0.06 mg/kg/day and temsirolimus at a dose of 20 mg/kg/day. Treatments started on day 21 post-implantation.

FIG. 29. Tumor weight evolution (mean±SEM) of NCI-H-460 tumors in mice treated with Control (vehicle), aplidine, temsirolimus or aplidine plus temsirolimus. Aplidine was administered at a dose of 0.06 mg/kg/day and temsirolimus at a dose of 10 mg/kg/day. Treatments started on day 7 post-implantation.

FIG. 30. Tumor weight evolution (mean±SEM) of NCI-H-460 tumors in mice treated with Control (vehicle), aplidine, temsirolimus or aplidine plus temsirolimus. Aplidine was administered at a dose of 0.06 mg/kg/day and temsirolimus at a dose of 20 mg/kg/day. Treatments started on day 7 post-implantation.

FIG. 31. Tumor weight evolution (mean±SEM) of HepG2 tumors in mice treated with Control (vehicle), aplidine, sorafenib or aplidine plus sorafenib. Aplidine was administered at a dose of 0.06 mg/kg/day and sorafenib at a dose of 30 mg/kg/day. Treatments started on day 19 post-implantation.

FIG. 32. Tumor weight evolution (mean±SEM) of HepG2 tumors in mice treated with Control (vehicle), aplidine, sorafenib or aplidine plus sorafenib. Aplidine was administered at a dose of 0.06 mg/kg/day and sorafenib at a dose of 60 mg/kg/day. Treatments started on day 19 post-implantation.

FIG. 33. Tumor weight evolution (mean±SEM) of LOX-IMVI tumors in mice treated with Control (vehicle), aplidine, sorafenib or aplidine plus sorafenib. Aplidine was administered at a dose of 0.06 mg/kg/day and sorafenib at a dose of 60 mg/kg/day. Treatments started on day 11 post-implantation.

FIG. 34. Tumor weight evolution (mean±SEM) of LOX-IMVI tumors in mice treated with Control (vehicle), aplidine, sorafenib or aplidine plus sorafenib. Aplidine was administered at a dose of 0.06 mg/kg/day and sorafenib at a dose of 30 mg/kg/day. Treatments started on day 11 post-implantation.

### DETAILED DESCRIPTION OF THE INVENTION

We surprisingly found that the anticancer activity of sorafenib, sunitinib, and temsirolimus is greatly enhanced when each of them is individually combined with aplidine. Thus, the present invention is directed to provide an efficacious treatment of cancer based on the combination of aplidine with another anticancer drug selected from sorafenib, sunitinib, and temsirolimus and mixtures thereof.

By “cancer” it is meant to include tumors, neoplasias, and any other malignant tissue or cells.

In another aspect, the invention relates to synergistic combinations employing aplidine, or a pharmaceutically acceptable salt thereof, and another anticancer drug selected from sorafenib, sunitinib, and temsirolimus, or a pharmaceutically acceptable salt thereof. Such synergistic combinations can be obtained by application of the methodology described herein, including those illustrated in Examples 1 to 7 and analyzing the results for synergistic combinations.

The term “combination” as used throughout the specification, is meant to encompass the administration of the therapeutic agents in the same or separate pharmaceutical formulations, and at the same time or at different times. If the therapeutic agents are administered at different times they should be administered sufficiently close in time to provide for the synergistic response to occur.

In another aspect, the invention is directed to the use of aplidine, or a pharmaceutically acceptable salt thereof, for the manufacture of a medicament for an effective treatment of cancer by combination therapy employing aplidine, or a pharmaceutically acceptable salt thereof, with another anticancer drug selected from sorafenib, sunitinib, and temsirolimus, or a pharmaceutically acceptable salt thereof.

In a related aspect, the invention is directed to the use of an anticancer drug selected from sorafenib, sunitinib, and temsirolimus, or a pharmaceutically acceptable salt thereof, for the manufacture of a medicament for an effective treatment of cancer by combination therapy employing sorafenib, sunitinib, or temsirolimus, or a pharmaceutically acceptable salt thereof, with aplidine, or a pharmaceutically acceptable salt thereof.

In a further aspect, the present invention is directed to a method of treating cancer comprising administering to a patient in need of such treatment a therapeutically effective amount of aplidine, or a pharmaceutically acceptable salt thereof, in combination with a therapeutically effective amount of another anticancer drug selected from sorafenib, sunitinib, and temsirolimus, or a pharmaceutically acceptable salt thereof.

The invention also provides a method of treating cancer comprising administering to a patient in need of such treatment a therapeutically effective amount of an anticancer drug selected from sorafenib, sunitinib, and temsirolimus, or a pharmaceutically acceptable salt thereof, in combination with a therapeutically effective amount of aplidine, or a pharmaceutically acceptable salt thereof.

Depending on the type of tumor and the development stage of the disease, anticancer effects of the methods of treatment of the present invention include, but are not limited to, inhibition of tumor growth, tumor growth delay, regression of tumor, shrinkage of tumor, increased time to regrowth of tumor on cessation of treatment, slowing of disease progression, and prevention of metastasis. It is expected that when a method of treatment of the present invention is administered to a patient, such as a human patient, in need of such treatment, said method of treatment will produce an effect, as measured by, for example, the extent of the anticancer effect, the response rate, the time to disease progression, or the survival rate. In particular, the methods of treatment of the invention are suited for human patients, especially those who are relapsing or refractory to previous chemotherapy. First line therapy is also envisaged.

As mentioned above, aplidine is a cyclic depsipeptide with the following structure:

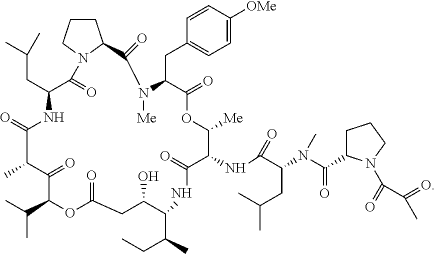

The term “aplidine” is intended here to cover any pharmaceutically acceptable salt, ester, solvate, hydrate, prodrug, or any other compound which, upon administration to the patient is capable of providing (directly or indirectly) the compounds as described herein. However, it will be appreciated that non-pharmaceutically acceptable salts also fall within the scope of the invention since these may be useful in the preparation of pharmaceutically acceptable salts. The preparation of salts, esters, solvates, hydrates, and prodrugs can be carried out by methods known in the art.

Any compound that is a prodrug of aplidine is within the scope and spirit of the invention. The term “prodrug” is used in its broadest sense and encompasses those derivatives that are converted in vivo to aplidine. The prodrug can hydrolyze, oxidize, or otherwise react under biological conditions to provide aplidine. Such derivatives would readily occur to those skilled in the art, and include, for example, compounds where a free hydroxy group is converted into an ester derivative.

Any compound referred to herein is intended to represent such specific compound as well as certain variations or forms. In particular, compounds referred to herein may have asymmetric centers and therefore exist in different enantiomeric forms. All optical isomers and stereoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof. Particularly, the compounds of the present invention may include enantiomers depending on their asymmetry or diastereoisomers. Stereoisomerism about the double bond is also possible, therefore in some cases the molecule could exist as (E)-isomer or (Z)-isomer. If the molecule contains several double bonds, each double bond will have its own stereoisomerism, that could be the same or different than the stereoisomerism of the other double bonds of the molecule. The single isomers and mixtures of isomers fall within the scope of the present invention.

Furthermore, compounds referred to herein may exist as geometric isomers (i.e., cis and trans isomers), as tautomers, or as atropisomers. Specifically, the term tautomer refers to one of two or more structural isomers of a compound that exist in equilibrium and are readily converted from one isomeric form to another. Common tautomeric pairs are amine-imine, amide-imide, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

Aplidine for use in accordance of the present invention may be prepared following a synthetic process such as those disclosed in WO 02/02596, WO 01/76616, and WO 2004/084812, which are incorporated herein by reference.

Pharmaceutical compositions of aplidine that can be used include solutions, suspensions, emulsions, lyophilised compositions, etc., with suitable excipients for intravenous administration. Preferably, aplidine may be supplied and stored as a sterile lyophilized product, comprising aplidine and excipients in a formulation adequate for therapeutic use. In particular a formulation comprising mannitol is preferred. Further guidance on aplidine formulations is given in WO 99/42125 which is incorporated herein by reference in its entirety.

Administration of aplidine, or pharmaceutical compositions thereof, is preferably by intravenous infusion. We prefer that infusion times of up to 72 hours are used, more preferably 1 to 24 hours, with about 1, about 3 or about 24 hours most preferred. Short infusion times which allow treatment to be carried out without an overnight stay in hospital are especially desirable. However, infusion may be around 24 hours or even longer if required. Infusion may be carried out at suitable intervals with varying patterns, illustratively once a week, twice a week, or more frequently per week, repeated each week optionally with gaps of typically one or several weeks.

Sorafenib is a kinase inhibitor with the following structural formula:

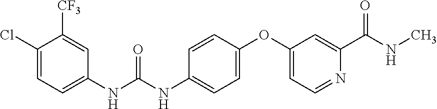

This drug is being marketed in the form of its tosylate salt with the trade name NEXAVAR®. This drug is currently indicated for the treatment of certain types of cancer, specifically for hepatocellular carcinoma and renal cell carcinoma. As single agent, the recommended daily dose, given orally, is 400 mg taken twice daily without food (at least 1 hour before or 2 hours after a meal), and treatment should continue until de patient is no longer clinically benefiting from therapy or until unacceptable toxicity occurs. Information about this drug is available on the website www.nexavar.com and the extensive literature on sorafenib.

Sorafenib was shown to inhibit multiple intracellular (CRAF, BRAF and mutant BRAF) and cell surface kinases (KIT, FLT-3, RET, VEGFR-1, VEGFR-2, VEGFR-3, and PDGFR-β) (Wilhelm S M et al. Cancer Res. 2004, 64, 7099-7109). Several of these kinases are thought to be involved in tumor cell signaling, angiogenesis, and apoptosis. Sorafenib inhibited tumor growth and angiogenesis of human hepatocellular carcinoma and renal cell carcinoma, and several other human tumor xenografts in immunocompromised mice.

Sunitinib is a multi-kinase inhibitor with the following structural formula:

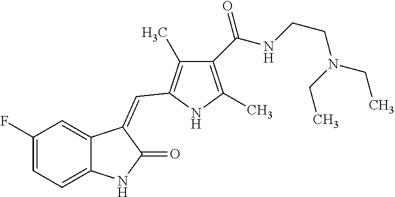

This drug is being marketed in the form of its malate salt with the trade name SUTENT®. This drug is currently indicated for the treatment of certain types of cancer, specifically for gastrointestinal stromal tumor (GIST) and renal cell carcinoma. As single agent, the recommended dose is one 50 mg oral dose taken once daily, on a schedule of 4 weeks on treatment followed by 2 weeks off. Dose increase or reduction of 12.5 mg increments is recommended based on individual safety and tolerability. Information about this drug is available on the website www.sutent.com and the extensive literature on sunitinib.

Sunitinib inhibits multiple receptor tyrosine kinases (RTKs), some of which are implicated in tumor growth, pathologic angiogenesis, and metastatic progression of cancer. Sunitinib was evaluated for its inhibitory activity against a variety of kinases (>80 kinases) and was identified as an inhibitor of platelet-derived growth factor receptors (PDGFRα and PDGFRβ), vascular endothelial growth factor receptors (VEGFR1, VEGFR2 and VEGFR3), stem cell factor receptor (KIT), Fms-like tyrosine kinase-3 (FLT3), colony stimulating factor receptor Type 1 (CSF-1R), and the glial cell-line derived neurotrophic factor receptor (RET) (Bergers G et al. J. Clin. Invest. 2003, 111, 1287-1295). Sunitinib inhibition of the activity of these RTKs has been demonstrated in biochemical and cellular assays, and inhibition of function has been demonstrated in cell proliferation assays. The primary metabolite exhibits similar potency compared to sunitinib in biochemical and cellular assays.

Sunitinib inhibited the phosphorylation of multiple RTKs (PDGFRβ, VEGFR2, KIT) in tumor xenografts expressing RTK targets in vivo and demonstrated inhibition of tumor growth or tumor regression and/or inhibited metastases in some experimental models of cancer. Sunitinib demonstrated the ability to inhibit growth of tumor cells expressing dysregulated target RTKs (PDGFR, RET, or KIT) in vitro and to inhibit PDGFRβ- and VEGFR2-dependent tumor angiogenesis in vivo.

Temsirolimus is an inhibitor of mTOR (mammalian target of rapamycin) with the following structural formula:

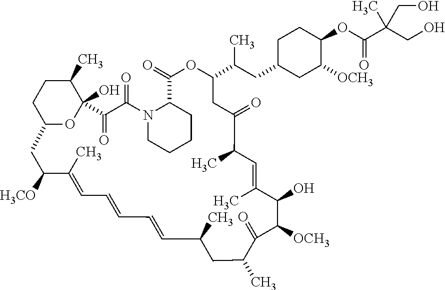

This drug is being marketed with the trade name TORISEL®, and it is currently indicated for the treatment of renal cell carcinoma. As single agent, the recommended dose is 25 mg infused over 30-60 minute period once a week, and treatment should continue until disease progression or unacceptable toxicity occurs. Information about this drug is available on the website www.torisel.com and the extensive literature on temsirolimus.

Temsirolimus binds to an intracellular protein (FKBP-12), and the protein-drug complex inhibits the activity of mTOR that controls cell division. Inhibition of mTOR activity resulted in a G1 growth arrest in treated tumor cells. When mTOR was inhibited, its ability to phosphorylate p70S6k and S6 ribosomal protein, which are downstream of mTOR in the PI3 kinase/AKT pathway was blocked. In in vitro studies using renal cell carcinoma cell lines, temsirolimus inhibited the activity of mTOR and resulted in reduced levels of the hypoxia-inducible factors HIF-1 and HIF-2 alpha, and the vascular endothelial growth factor.

Pharmaceutically acceptable salts of any drug referred to herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In addition, any drug referred to herein may be in crystalline form either as free compound or as solvates (e.g. hydrates) and it is intended that both forms are within the scope of the present invention. Methods of solvation are generally known within the art.

Aplidine, or a pharmaceutically acceptable salt thereof, and the other anticancer drug selected from sorafenib, sunitinib, and temsirolimus, or a pharmaceutically acceptable salt thereof, may be provided as separate medicaments for administration at the same time or at different times. Preferably, aplidine and the other anticancer drug are provided as separate medicaments for administration at different times. When administered separately and at different times, either aplidine or the other anticancer drug, may be administered first. In addition, both drugs can be administered in the same day or at different days, and they can be administered using the same schedule or at different schedules during the treatment cycle. Thus, the pharmaceutical compositions of the present invention may comprise all the components (drugs) in a single pharmaceutically acceptable formulation. Alternatively, the components may be formulated separately and administered in combination with one another. Various pharmaceutically acceptable formulations well known to those of skill in the art can be used in the present invention. Additionally, the drugs of the combination may be given using different administration routes. For instance, one of the drugs may be in a form suitable for oral administration, for example as a tablet or capsule, and the other one in a form suitable for parenteral injection (including intravenous, subcutaneous, intramuscular, intravascular or infusion), for example as a sterile solution, suspension or emulsion. Alternatively, both drugs may be given by the same administration route. Selection of an appropriate formulation for use in the present invention can be performed routinely by those skilled in the art based upon the mode of administration and the solubility characteristics of the components of the composition

The correct dosage of the compounds of the combination will vary according to the particular formulation, the mode of application, and the particular site, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose. Further guidance for the administration of aplidine is given in WO 01/35974 which is incorporated herein by reference in its entirety.

In another aspect, the present invention is directed to a kit for administering aplidine in combination with another anticancer drug selected from sorafenib, sunitinib, and temsirolimus in the treatment of cancer, comprising a supply of aplidine, or a pharmaceutically acceptable salt thereof, in dosage units for at least one cycle, and printed instructions for the use of both drugs in combination.

In a related aspect, the present invention is directed to a kit for administering an anticancer drug selected from sorafenib, sunitinib, and temsirolimus in combination with aplidine in the treatment of cancer, comprising a supply of the anticancer drug selected from sorafenib, sunitinib, and temsirolimus, or a pharmaceutically acceptable salt thereof, in dosage units for at least one cycle, and printed instructions for the use of both drugs in combination.

In a related aspect, the present invention is directed to a kit for administering aplidine in combination with another anticancer drug selected from sorafenib, sunitinib, and temsirolimus in the treatment of cancer, comprising a supply of aplidine, or a pharmaceutically acceptable salt thereof, in dosage units for at least one cycle, a supply of an anticancer drug selected from sorafenib, sunitinib, and temsirolimus, or a pharmaceutically acceptable salt thereof, in dosage units for at least one cycle, and printed instructions for the use of both drugs in combination.

In another aspect, the present invention also provides a pharmaceutical composition comprising aplidine, or a pharmaceutically acceptable salt thereof, and a pharmaceutically acceptable carrier, for use in combination with another anticancer drug selected from sorafenib, sunitinib, and temsirolimus, or a pharmaceutically acceptable salt thereof, in the treatment of cancer.

In a further aspect, the present invention also provides a pharmaceutical composition comprising an anticancer drug selected from sorafenib, sunitinib, and temsirolimus, or a pharmaceutically acceptable salt thereof, and a pharmaceutically acceptable carrier, for use in combination with aplidine, or a pharmaceutically acceptable salt thereof, in the treatment of cancer.

In addition, the present invention also provides a pharmaceutical composition comprising aplidine, or a pharmaceutically acceptable salt thereof, an anticancer drug selected from sorafenib, sunitinib, and temsirolimus, or a pharmaceutically acceptable salt thereof, and a pharmaceutically acceptable carrier, for use in the treatment of cancer.

In another aspect, the invention further provides for the use of aplidine, or a pharmaceutically acceptable salt thereof, in the preparation of a composition for use in combination with another anticancer drug selected from sorafenib, sunitinib, and temsirolimus, or a pharmaceutically acceptable salt thereof, in the treatment of cancer.

In a related aspect, the invention further provides for the use of an anticancer drug selected from sorafenib, sunitinib, and temsirolimus, or a pharmaceutically acceptable salt thereof, in the preparation of a composition for use in combination with aplidine, or a pharmaceutically acceptable salt thereof, in the treatment of cancer.

And in a further aspect, the invention also provides for the use of aplidine, or a pharmaceutically acceptable salt thereof, and another anticancer drug selected from sorafenib, sunitinib, and temsirolimus, or a pharmaceutically acceptable salt thereof, in the preparation of a composition for use in the treatment of cancer.

In another aspect, the invention further provides for the use of aplidine, or a pharmaceutically acceptable salt thereof, for the manufacture of a medicament for the treatment of cancer, in combination therapy with another anticancer drug selected from sorafenib, sunitinib, and temsirolimus, or a pharmaceutically acceptable salt thereof.

In a related aspect, the invention further provides for the use of an anticancer drug selected from sorafenib, sunitinib, and temsirolimus, or a pharmaceutically acceptable salt thereof, for the manufacture of a medicament for the treatment of cancer, in combination therapy with aplidine, or a pharmaceutically acceptable salt thereof.

In a related aspect, the invention further provides for the use of aplidine, or a pharmaceutically acceptable salt thereof, in combination with another anticancer drug selected from sorafenib, sunitinib, and temsirolimus, or a pharmaceutically acceptable salt thereof, for the manufacture of a medicament for the treatment of cancer.

In another aspect, the invention further provides for the use of aplidine, or a pharmaceutically acceptable salt thereof, for the treatment of cancer, in combination therapy with another anticancer drug selected from sorafenib, sunitinib, and temsirolimus, or a pharmaceutically acceptable salt thereof.

In a related aspect, the invention further provides for the use of an anticancer drug selected from sorafenib, sunitinib, and temsirolimus, or a pharmaceutically acceptable salt thereof, for the treatment of cancer, in combination therapy with aplidine, or a pharmaceutically acceptable salt thereof.

In another aspect, the invention further provides for the use of aplidine, or a pharmaceutically acceptable salt thereof, in combination with another anticancer drug selected from sorafenib, sunitinib, and temsirolimus, or a pharmaceutically acceptable salt thereof, for the treatment of cancer.

In another aspect, the invention further provides for the use of aplidine, or a pharmaceutically acceptable salt thereof, as a medicament, in combination therapy with another anticancer drug selected from sorafenib, sunitinib, and temsirolimus, or a pharmaceutically acceptable salt thereof.

In a related aspect, the invention further provides for the use of an anticancer drug selected from sorafenib, sunitinib, and temsirolimus, or a pharmaceutically acceptable salt thereof, as a medicament, in combination therapy with aplidine, or a pharmaceutically acceptable salt thereof.

In another aspect, the invention further provides for the use of aplidine, or a pharmaceutically acceptable salt thereof, in combination with another anticancer drug selected from sorafenib, sunitinib, and temsirolimus, or a pharmaceutically acceptable salt thereof, as a medicament.

In another aspect, the invention further provides for the use of aplidine, or a pharmaceutically acceptable salt thereof, as a medicament for the treatment of cancer, in combination therapy with another anticancer drug selected from sorafenib, sunitinib, and temsirolimus, or pharmaceutically acceptable salt thereof.

In a related aspect, the invention further provides for the use of an anticancer drug selected from sorafenib, sunitinib, and temsirolimus, or a pharmaceutically acceptable salt thereof, as a medicament for the treatment of cancer, in combination therapy with aplidine, or pharmaceutically acceptable salt thereof.

In another aspect, the invention further provides for the use of aplidine, or a pharmaceutically acceptable salt thereof, in combination with another anticancer drug selected from sorafenib, sunitinib, and temsirolimus, or a pharmaceutically acceptable salt thereof, as a medicament for the treatment of cancer.

In another aspect, the invention provides aplidine, or a pharmaceutically acceptable salt thereof, for the treatment of cancer comprising administering a therapeutically effective amount of aplidine, or a pharmaceutically acceptable salt thereof, in combination with a therapeutically effective amount of an anticancer drug selected from sorafenib, sunitinib, and temsirolimus, or a pharmaceutically acceptable salt thereof.

In a related aspect, the invention further provides an anticancer drug selected from sorafenib, sunitinib, and temsirolimus, or a pharmaceutically acceptable salt thereof, for the treatment of cancer comprising administering a therapeutically effective amount of an anticancer drug selected from sorafenib, sunitinib, and temsirolimus, or a pharmaceutically acceptable salt thereof, in combination with a therapeutically effective amount of aplidine, or a pharmaceutically acceptable salt thereof.

In another aspect, the invention provides for the treatment of cancer comprising the administration of a therapeutically effective amount of aplidine, or pharmaceutically acceptable salt thereof, in combination with the administration of a therapeutically effective amount of another anticancer drug selected from sorafenib, sunitinib, and temsirolimus, or a pharmaceutically acceptable salt thereof, wherein the combination may be administered together or separately. In preferred embodiments of the invention aplidine, or pharmaceutically acceptable salts thereof, and another anticancer drug selected from sorafenib, sunitinib and temsirolimus, or pharmaceutically acceptable salts thereof, are administered in synergistically effective amounts.

Preferably, the combination of aplidine, or a pharmaceutically acceptable salt thereof, with another anticancer drug selected from sorafenib, sunitinib, and temsirolimus, or a pharmaceutically acceptable salt thereof, is used for the treatment of renal carcinoma, hepatocarcinoma (also known as hepatoma), melanoma, breast cancer, lung cancer, pancreatic cancer, neuroblastoma, and gastrointestinal stromal tumor (GIST). Specially preferred is the use of the combination for the treatment of renal carcinoma, hepatocarcinoma, melanoma, NSCLC, and breast cancer.

In one embodiment, the combination of aplidine, or a pharmaceutically acceptable salt thereof, and another anticancer drug selected from sorafenib, sunitinib, and temsirolimus, or a pharmaceutically acceptable salt thereof, inhibits tumor growth or reduce the size of a tumor in vivo. In particular, the combination inhibits in vivo growth of carcinoma cells, sarcoma cells, leukemia cells, lymphoma cells and myeloma cells. Preferably, the combination inhibits in vivo growth of renal carcinoma cells, hepatocarcinoma cells, melanoma cells, breast cancer cells, lung cancer cells, pancreatic cancer cells, neuroblastoma cells, and GIST cells. Specifically, the combination inhibits in vivo growth of human renal carcinoma cells, human hepatocarcinoma cells, human melanoma cells, and human NSCLC cells. Similarly, the combination reduces the size of carcinoma, sarcoma, leukemia, lymphoma and myeloma tumors in vivo. Preferably, the combination reduces the size of renal carcinoma, hepatocarcinoma, melanoma, breast cancer, lung cancer, pancreatic cancer, neuroblastoma, and GIST in vivo. Specifically, the combination reduces the size of human renal tumors, human hepatocarcinomas, human melanomas, and human NSCL carcinomas in vivo.

For example, the combination inhibits tumor growth or reduces the size of human cancer xenografts, particularly human renal tumor xenografts, human hepatocarcinoma xenografts, human melanoma xenografts, and human NSCLC xenografts, in animal models. A reduced growth or reduced size of human cancer xenografts in animal models administered with the combination supports the combination of aplidine, or a pharmaceutically acceptable salt thereof, and another anticancer drug selected from sorafenib, sunitinib, and temsirolimus, or a pharmaceutically acceptable salt thereof, as being effective for treating a patient with that particular type of cancer. In addition, a low level of toxicity in animal models provides for the selective cytotoxic activity of the combination against cancer cells.

According to an embodiment of the invention, tumor growth inhibition is assessed comparing the mean tumor weight of the treatment combining the two drugs (aplidine and sorafenib, aplidine and sunitinib, or aplidine and temsirolimus) with those of sorafenib, sunitinib, or temsirolimus monotherapy treatment, respectively. Additionally, the definition and criteria for the evaluation of potentiation and the degree of additivity for the combination therapy are as follows:

Potentiation can be determined when the response of the combination therapy is greater than the best response of the most active drug administered as single agent (monotherapy) on the same schedule and dose as used in the combination therapy.

Additivity is determined by comparing the % of tumor growth inhibition of the monotherapy treatments versus those of the combination treatment as follows:
1. Determination of the % of tumor growth inhibition, as 100-% T/C, for each of the drugs administered as monotherapy at the doses used in the combinations. % T/C is obtained by comparing the mean tumor weight in the treatment groups (T) to the mean tumor weight in the control group (C) (T/C×100%).
2. The two scores are added together to determine the “expected response” if each agent produced the same response as it does when administered as monotherapy.
3. This “expected response” is subtracted from the % of tumor growth inhibition determined for the combination therapy group:
   a. A negative number means that the effect of combining the two drugs is less than additive.
   b. If the resulting number is close to zero, the effect of combining the two drugs is determined as additive.
   c. A positive number means that the effect of combining the two drugs is greater than additive.

Accordingly, a greater than additive effect of the combination treatment corresponds to a synergistic effect, wherein the effect of the combination of the two drugs is therapeutically superior to that expected in view of the effect of each of the drugs when given alone.

Therefore, in another aspect, the invention provides for a method for reducing the size of a tumor, comprising administering an effective amount of aplidine, or a pharmaceutically acceptable salt thereof, in combination with another anticancer drug selected from sorafenib, sunitinib, and temsirolimus, or a pharmaceutically acceptable salt thereof.

In a related aspect, the invention provides for a method for reducing the size of a tumor, comprising administering an effective amount of an anticancer drug selected from sorafenib, sunitinib, and temsirolimus, or a pharmaceutically acceptable salt thereof, in combination with aplidine, or a pharmaceutically acceptable salt thereof.

In a related aspect, the invention provides for a method for reducing the size of a tumor, comprising administering an effective combination of aplidine, or a pharmaceutically acceptable salt thereof, and an anticancer drug selected from sorafenib, sunitinib, and temsirolimus, or a pharmaceutically acceptable salt thereof, together or separately.

In another aspect, the invention provides for a method for inhibiting tumor growth, comprising administering an effective amount of aplidine, or a pharmaceutically acceptable salt thereof, in combination with another anticancer drug selected from sorafenib, sunitinib, and temsirolimus, or a pharmaceutically acceptable salt thereof.

In a related aspect, the invention provides for a method for inhibiting tumor growth, comprising administering an effective amount of an anticancer drug selected from sorafenib, sunitinib, and temsirolimus, or a pharmaceutically acceptable salt thereof, in combination with aplidine, or a pharmaceutically acceptable salt thereof.

In a related aspect, the invention provides for a method for inhibiting tumor growth, comprising administering an effective combination of aplidine, or a pharmaceutically acceptable salt thereof, and an anticancer drug selected from sorafenib, sunitinib, and temsirolimus, or a pharmaceutically acceptable salt thereof, together or separately.

The following examples further illustrate the invention. The examples should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

### Example 1

### In Vivo Studies to Determine the Effect of Aplidine in Combination with Sorafenib in Human Renal Tumor Xenografts

The aim of these studies was to evaluate the ability of aplidine to potentiate the antitumoral activity of sorafenib by using three xenograft models of human renal cancer.

Female athymic nude mice (Harlan Sprague Dawley, Madison, Wis.) were utilized for all experiments. Mice were implanted with tumor fragments or a cell suspension when mice were 5 weeks of age. Animals were housed in ventilated rack caging, 5 mice per cage, with food and water ad libitum. The mice were acclimated for 1 week prior to being implanted with tumor fragments or cell suspensions. The Vehicle Control group contained 15 mice and the treated groups had each 10 mice/group.

The tumor models used in these studies were CAKI-1 cell line, which is a human kidney clear carcinoma cell line obtained from the ATCC (Manassas, Va.), MRI-H-121 cell line, which is a human kidney carcinoma cell line originally obtained from the DCT Tumor Bank, and A498 cell line, which is a human kidney carcinoma cell line obtained from the ATCC (Manassas, Va.).

For MRI-H-121 and CAKI-1 assays, animals were implanted subcutaneously (SC) on the right flank, using a trocar, with 3 mm³ of tissue tumor fragments, from an in vivo transplantable line passage 1, using sterile Earle's Balanced Salt solution as a wetting agent. Bacterial cultures taken at the implantation time were negative for contamination at both 24 and 48 hours post-implant.

A498 cells were grown in MEM, 2 mM L-glutamine and 10% FBS. Cells from in vitro passage 4-20 were implanted SC into study mice: 5×10⁶ cells/mouse in 0.2 ml 50% Matrigel/50% medium without antibiotics or serum, using a 23 G needle and 1 cc syringe. Matrigel is a biological extracellular matrix that is liquid at 4° C. and solid at 37° C., and it promotes tumor growth by maintaining the cells in close association in a localized area. Bacterial cultures were performed on aliquots of the cells prepared for implantation. All cultures were negative for bacterial contamination at both 24 and 48 hours post-implant.

Tumor measurements were determined by using Vernier calipers. The formula to calculate volume for a prolate ellipsoid was used to estimate tumor volume (mm³) from 2-dimensional tumor measurements: Tumor volume (mm³)=[L×W²]÷2, where L is the length and it is the longest diameter in mm, and W is the width and it is the shortest diameter in mm of a tumor. Assuming unit density, volume was converted to weight (i.e., 1 mm³=1 mg). When tumors reached an appropriated volume, within the size range of 143±2 mg for MRI-H-121, 259±32 mg for CAKI-1, and 111±3 mg for A498 (mean±SD), mice were randomized into the treatment and control groups based on tumor weight by using LabCat® In Life module V 8.0 SP1 tumor tracking and measurement software.

The treatments of MRI-H-121-tumor-bearing mice were initiated on DPI (Day Post Implantation) 14, of CAKI-1 on DPI 13 and of A498 DPI 26. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. Then the aplidine solution in CEW was diluted in 0.9% Saline to the dosing formulation concentrations, being the final proportion of CEW of 0.18/0.18/0.84.

Sorafenib was provided in the form of a 200 mg reddish colored tablet containing sorafenib in the form of its tosylate salt. The sorafenib solution was made by solving the tablet in Cremophor EL/ethanol (50/50) to a concentration of 50 mg/ml. Then the solution was diluted in water for infusion (wfi) to a final proportion of CEW of (12.5, 12.5, 75). The sorafenib solution in CEW was diluted in wfi to the dosing formulation concentrations.

Study groups and treatment regimens for the three xenograft models are listed in table I.

TABLE I

| | N of | | | | Sched- | Test |
|---|---|---|---|---|---|---|
| Group | Animals | Dose | | Route | ule | material |
| G1 | 15 | 10 | ml/kg/day | IP | Qdx9x2 | CEW (0.18/ |
| (Control | | | | | | 0.18/0.84) |
| group) | | 10 | ml/kg/day | PO | Qdx21 | CEW (12.5, |
| | | | | | | 12.5, 75) |
| G2 | 10 | 0.060 | mg/kg/day | IP | Qdx9x2 | aplidine |
| G3 | 10 | 0.040 | mg/kg/day | IP | Qdx9x2 | aplidine |
| G4 | 10 | 60 | mg/kg/day | PO | Qdx21 | sorafenib |
| G5 | 10 | 30 | mg/kg/day | PO | Qdx21 | sorafenib |
| G6 | 10 | 0.060 | mg/kg/day | IP | Qdx9x2 | aplidine |
| | | 60 | mg/kg/day | PO | Qdx21 | sorafenib |
| G7 | 10 | 0.060 | mg/kg/day | IP | Qdx9x2 | aplidine |
| | | 30 | mg/kg/day | PO | Qdx21 | sorafenib |
| G8 | 10 | 0.040 | mg/kg/day | IP | Qdx9x2 | aplidine |
| | | 60 | mg/kg/day | PO | Qdx21 | sorafenib |
| G9 | 10 | 0.040 | mg/kg/day | IP | Qdx9x2 | aplidine |
| | | 30 | mg/kg/day | PO | Qdx21 | sorafenib |

IP: Intraperitoneal administration; PO: Oral administration
Qdx9x2: Two cycles wherein test material is administered every day for 9 consecutive days with a rest period of 3 days between cycles
Qdx21: Administration of the test material every day for 21 consecutive days

Tumor size measurements were recorded twice weekly from the treatment initiation until the termination of the study. Tumor growth inhibition was assessed comparing the mean tumor weight between the two agents in combination (aplidine and sorafenib) against sorafenib mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were as follows:
Potentiation was determined when the response of the combination group was greater than the best response of the most active agent administered as single agent (monotherapy) on the same schedule and dose as used in the combination therapy.
Additivity was determined as discussed above by comparing the % of tumor growth inhibition of the monotherapy groups versus those of the combination group as follows:
   1. Determine the % of tumor growth inhibition, as 100−% T/C, for each of the drugs administered as monotherapy at the doses used in the combinations. % T/C was obtained by comparing the mean tumor weight in the treatment groups (T) to the mean tumor weight in the control group (C) (T/C×100%).
   2. The two scores were added together to determine the “expected response” if each agent produced the same response as it did when administered as monotherapy.
   3. This “expected response” was subtracted from the % of tumor growth inhibition determined for the combination therapy group:
      a. A negative number meant that the effect of combining the two drugs was less than additive.
      b. If the resulting number was close to zero, the effect of combining the two drugs was determined as additive.
      c. A positive number meant that the effect of combining the two drugs was greater than additive.

Accordingly, a greater than additive effect of the combination treatment corresponds to a synergistic effect, wherein the effect of the combination of the two drugs is therapeutically superior to that expected in view of the effect of each of the drugs when given alone.

Body weight effects were determined by comparing each mouse body weight measurement with the initial body weight (first day of treatment).

The NCI activity criterion of body weight loss >20% was used to gauge compound toxicity.

### Results in CAKI-1 Human Renal Tumor Xenograft

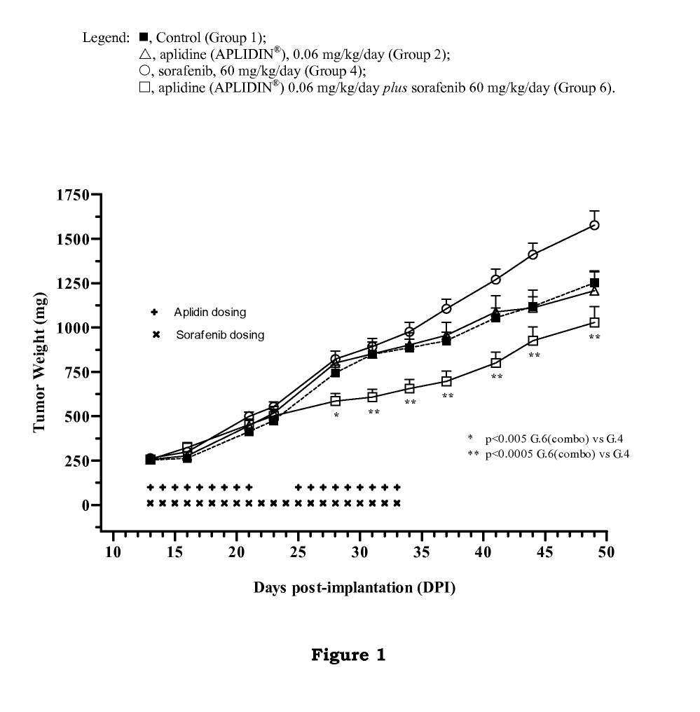

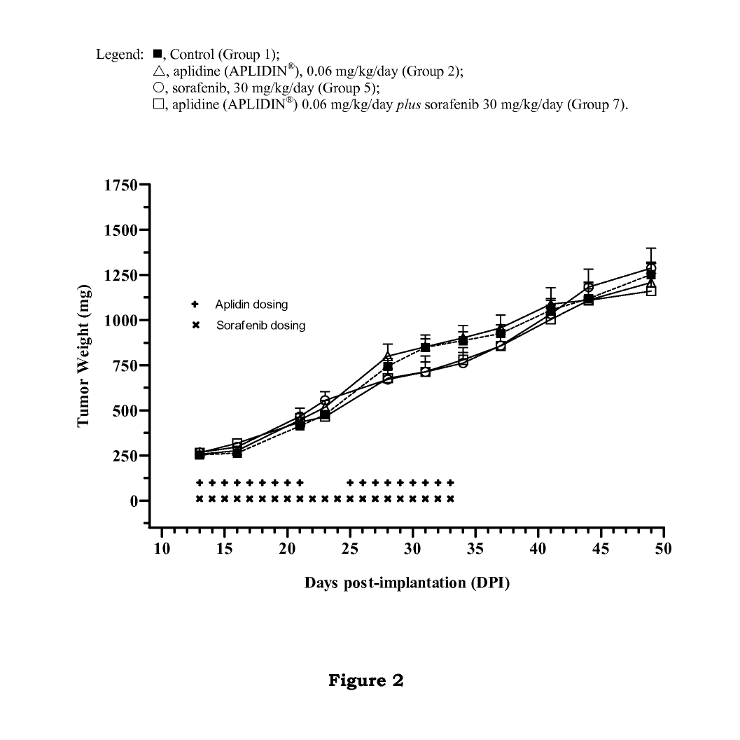

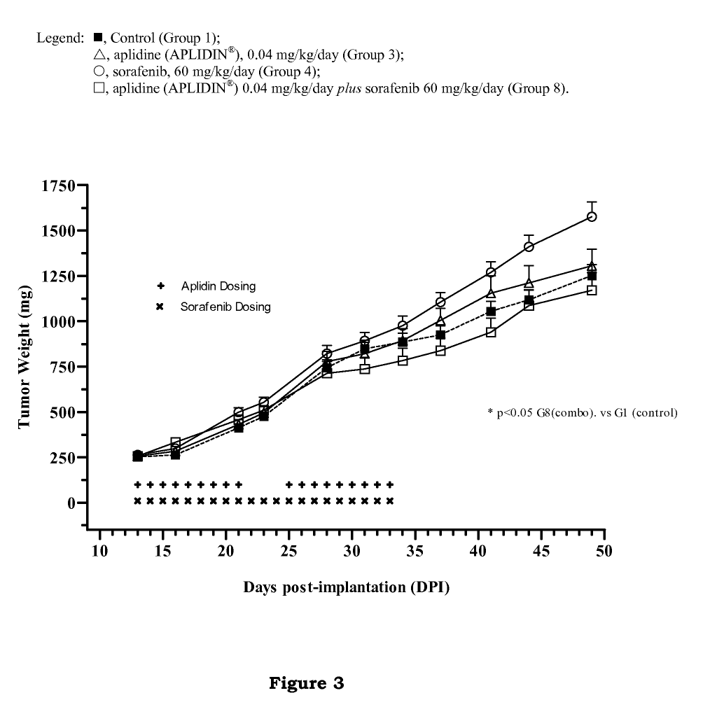

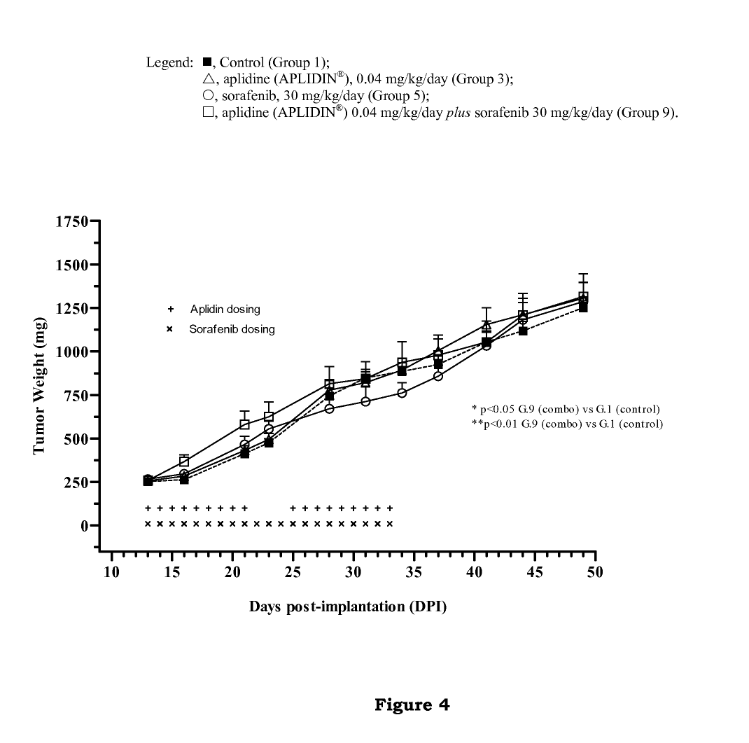

Table II reports the % T/C values obtained with each of the treatments and FIGS. 1-4 show the tumor weight evolution (mean±SEM) of CAKI-1 tumors in mice treated with control (vehicle), aplidine, sorafenib, or aplidine plus sorafenib at different doses.

TABLE II

| | % T/C on day | | | | | | |
|---|---|---|---|---|---|---|---|
| Group | 7 | 9 | 13 | 16 | 21 | 23 | 28 |
| G1 | — | — | — | — | — | — | — |
| (Control | | | | | | | |
| group) | | | | | | | |
| G2 | 103.9 | 113.8 | 101.6 | 105.7 | 108.5 | 109.0 | 107.5 |
| G3 | 115.8 | 124.7 | 101.5 | 108.2 | 104.9 | 104.3 | 104.6 |
| G4 | 100.6 | 128.9 | 104.5 | 114.0 | 121.2 | 116.6 | 110.5 |
| G5 | 116.6 | 130.2 | 105.4 | 113.4 | 113.0 | 117.1 | 90.3 |
| G6 | 120.7 | 111.5 | 101.7 | 123.2 | 109.9 | 105.8 | 78.8 |
| G7 | 113.3 | 115.4 | 104.7 | 121.4 | 105.6 | 97.7 | 91.0 |
| G8 | 93.9 | 119.8 | 101.4 | 126.8 | 111.2 | 107.4 | 95.9 |
| G9 | 113.2 | 122.0 | 102.5 | 140.2 | 140.9 | 131.6 | 109.6 |

% T/C on day

| Group | 31 | 34 | 37 | 41 | 44 | 49 |
|---|---|---|---|---|---|---|
| G1 | — | — | — | — | — | — |
| (Control | | | | | | |
| group) | | | | | | |
| G2 | 100.3 | 101.8 | 107.7 | 103.1 | 99.4 | 96.5 |
| G3 | 96.8 | 100.9 | 108.7 | 109.5 | 108.4 | 104.4 |
| G4 | 105.2 | 110.2 | 119.5 | 120.5 | 126.3 | 126.0 |
| G5 | 84.0 | 86.0 | 92.8 | 98.0 | 105.8 | 102.8 |
| G6 | 71.7 | 74.1 | 75.3 | 75.9 | 82.8 | 82.2 |
| G7 | 84.2 | 88.1 | 92.6 | 95.2 | 99.2 | 92.7 |
| G8 | 87.0 | 88.5 | 90.7 | 89.2 | 97.2 | 93.6 |
| G9 | 99.6 | 105.9 | 105.9 | 100.0 | 108.2 | 105.2 |

Table III shows the % of tumor growth inhibition of aplidine and sorafenib administered as single agents and in combination against CAKI-1 human renal tumor xenograft at a dose of 0.06 mg/kg/day of aplidine and 60 mg/kg/day of sorafenib. Additionally, the potentiation and the degree of additivity of the combination of aplidine with sorafenib at said doses are provided.

TABLE III

| | % Inhibition | | | Expected | Actual | Potent- | Degree of |
|---|---|---|---|---|---|---|---|
| Day | G2 | G4 | G6 | Response | Response | ation | Response |
| 13 | −1.6 | −4.5 | −1.7 | −6.1 | 4.3 | no | — |
| 16 | −5.7 | −14.0 | −23.2 | −19.7 | −3.5 | no | — |
| 21 | −8.5 | −21.2 | −9.9 | −29.7 | 19.8 | yes | Greater |
| | | | | | | | than |
| | | | | | | | additive |
| 23 | −9.0 | −16.6 | −5.8 | −25.6 | 19.8 | yes | Greater |
| | | | | | | | than |
| | | | | | | | additive |
| 28 | −7.5 | −10.5 | 21.2 | −18.0 | 39.2 | yes | Greater |
| | | | | | | | than |
| | | | | | | | additive |
| 31 | −0.3 | −5.2 | 28.3 | −5.5 | 33.8 | yes | Greater |
| | | | | | | | than |
| | | | | | | | additive |
| 34 | −1.8 | −10.2 | 25.9 | −12.0 | 37.9 | yes | Greater |
| | | | | | | | than |
| | | | | | | | additive |
| 37 | −7.7 | −19.5 | 24.7 | −27.2 | 51.9 | yes | Greater |
| | | | | | | | than |
| | | | | | | | additive |
| 41 | −3.1 | −20.5 | 24.1 | −23.6 | 47.7 | yes | Greater |
| | | | | | | | than |
| | | | | | | | additive |
| 44 | 0.6 | −26.3 | 17.2 | −25.7 | 42.9 | yes | Greater |
| | | | | | | | than |
| | | | | | | | additive |
| 49 | 3.5 | −26.0 | 17.8 | −22.4 | 40.2 | yes | Greater |
| | | | | | | | than |
| | | | | | | | additive |

Table IV shows the % of tumor growth inhibition of aplidine and sorafenib administered as single agents and in combination against CAKI-1 human renal tumor xenograft at a dose of 0.06 mg/kg/day of aplidine and 30 mg/kg/day of sorafenib. Additionally, the potentiation and the degree of additivity of the combination of aplidine with sorafenib at said doses are provided.

TABLE IV

| | % Inhibition | | | Expected | Actual | Potent- | Degree of |
|---|---|---|---|---|---|---|---|
| Day | G2 | G5 | G7 | Response | Response | ation | Response |
| 13 | −1.6 | −5.4 | −4.7 | −7.0 | 2.3 | no | — |
| 16 | −5.7 | −13.4 | −21.4 | −19.0 | −2.3 | no | — |
| 21 | −8.5 | −13.0 | −5.6 | −21.4 | 15.9 | yes | Greater |
| | | | | | | | than |
| | | | | | | | additive |
| 23 | −9.0 | −17.1 | 2.3 | −26.1 | 28.4 | yes | Greater |
| | | | | | | | than |
| | | | | | | | additive |
| 28 | −7.5 | 9.7 | 9.0 | 2.2 | 6.8 | yes | Additive |
| 31 | −0.3 | 16.0 | 15.8 | 15.7 | 0.2 | no | — |
| 34 | −1.8 | 14.0 | 11.9 | 12.3 | −0.4 | no | — |
| 37 | −7.7 | 7.2 | 7.4 | −0.5 | 7.9 | yes | Additive |
| 41 | −3.1 | 2.0 | 4.8 | −1.2 | 6.0 | yes | Additive |
| 44 | 0.6 | −5.8 | 0.8 | −5.2 | 6.0 | yes | Additive |
| 49 | 3.5 | −2.8 | 7.3 | 0.7 | 6.6 | yes | Additive |

Table V shows the % of tumor growth inhibition of aplidine and sorafenib administered as single agents and in combination against CAKI-1 human renal tumor xenograft at a dose of 0.04 mg/kg/day of aplidine and 60 mg/kg/day of sorafenib. Additionally, the potentiation and the degree of additivity of the combination of aplidine with sorafenib at said doses are provided.

TABLE V

| | % Inhibition | | | Expected | Actual | Potent- | Degree of |
|---|---|---|---|---|---|---|---|
| Day | G3 | G4 | G8 | Response | Response | ation | Response |
| 13 | −1.5 | −4.5 | −1.4 | −6.0 | 4.6 | yes | Additive |
| 16 | −8.2 | −14.0 | −26.8 | −22.2 | −4.6 | no | Less than |
| | | | | | | | additive |
| 21 | −4.9 | −21.2 | −11.2 | −26.1 | 14.8 | yes | Greater |
| | | | | | | | than |
| | | | | | | | additive |
| 23 | −4.3 | −16.6 | −7.4 | −20.9 | 13.5 | yes | Greater |
| | | | | | | | than |
| | | | | | | | additive |
| 28 | −4.6 | −10.5 | 4.1 | −15.0 | 19.1 | yes | Greater |
| | | | | | | | than |
| | | | | | | | additive |
| 31 | 3.2 | −5.2 | 13.0 | −2.0 | 15.0 | yes | Greater |
| | | | | | | | than |
| | | | | | | | additive |
| 34 | −0.9 | −10.2 | 11.5 | −11.1 | 22.6 | yes | Greater |
| | | | | | | | than |
| | | | | | | | additive |
| 37 | −8.7 | −19.5 | 9.3 | −28.3 | 37.6 | yes | Greater |
| | | | | | | | than |
| | | | | | | | additive |
| 41 | −9.5 | −20.5 | 10.8 | −30.0 | 40.8 | yes | Greater |
| | | | | | | | than |
| | | | | | | | additive |
| 44 | −8.4 | −26.3 | 2.8 | −34.7 | 37.5 | yes | Greater |
| | | | | | | | than |
| | | | | | | | additive |
| 49 | −4.4 | −26.0 | 6.4 | −30.4 | 36.8 | yes | Greater |
| | | | | | | | than |
| | | | | | | | additive |

When aplidine and sorafenib were administered as single agents (monotherapy) against CAKI-1 human renal tumor cell line, they were inactive. Additionally, these treatments did not cause a significant decline in mean body weight, and all mice gained weight by the end of the study.

However, when aplidine, at a dose of 0.060 mg/kg/day, was combined with sorafenib, at a dose of 60 mg/kg/day, a statistically significant inhibition of tumor growth was observed from day 28 through the end of the study on day 49. This combination resulted in a greater than additive potentiation of antitumor activity. Additionally, the combination of aplidine, at a dose of 0.040 mg/kg/day, with sorafenib, at a dose of 60 mg/kg/day, also resulted in a greater than additive potentiation of antitumor activity. The effect in all the cases was observed between days during the end of dosing period or immediately following the dosing period and lasting till the assay was terminated. Finally, combination therapy was well tolerated by the mice with no increased evidence of toxicity.

### Results in MRI-H-121 Human Renal Tumor Xenograft

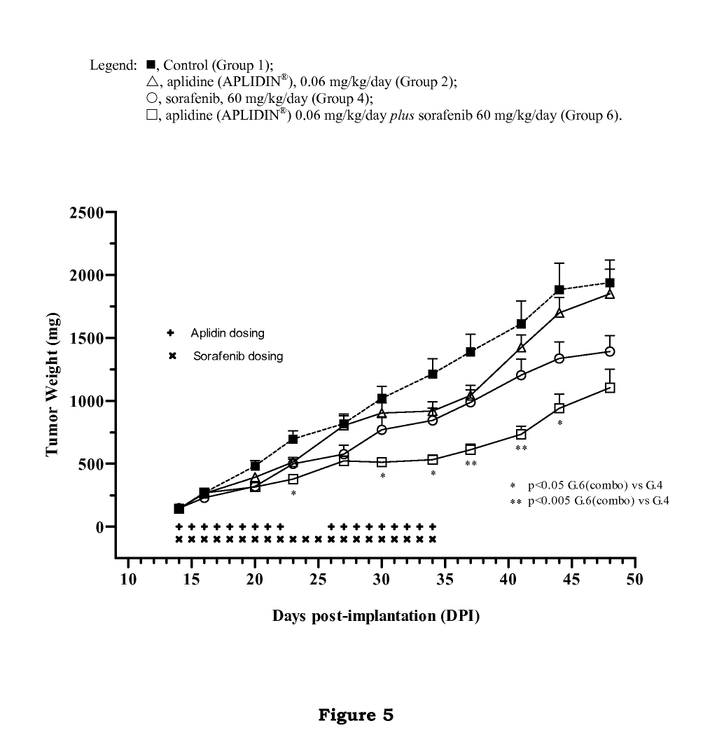

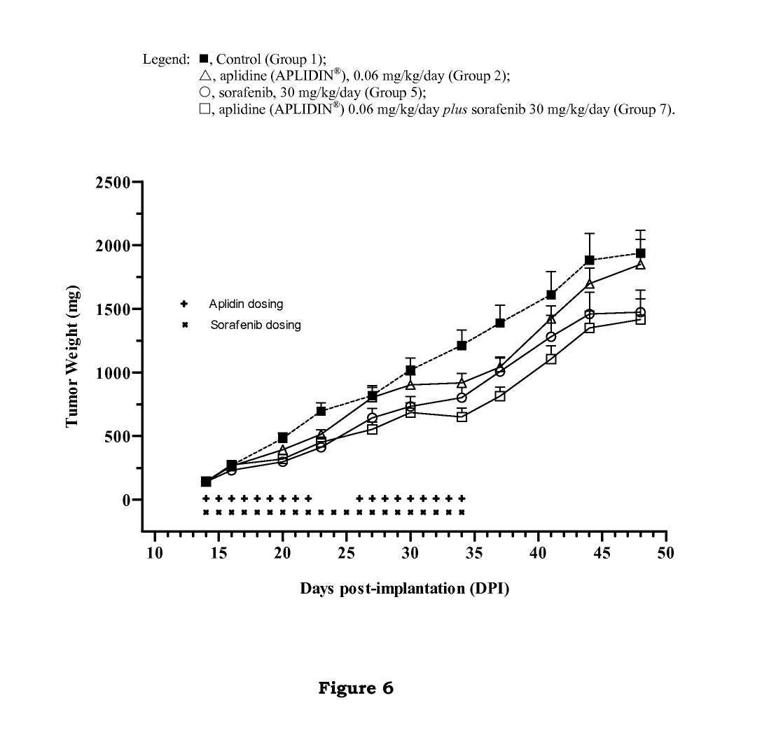

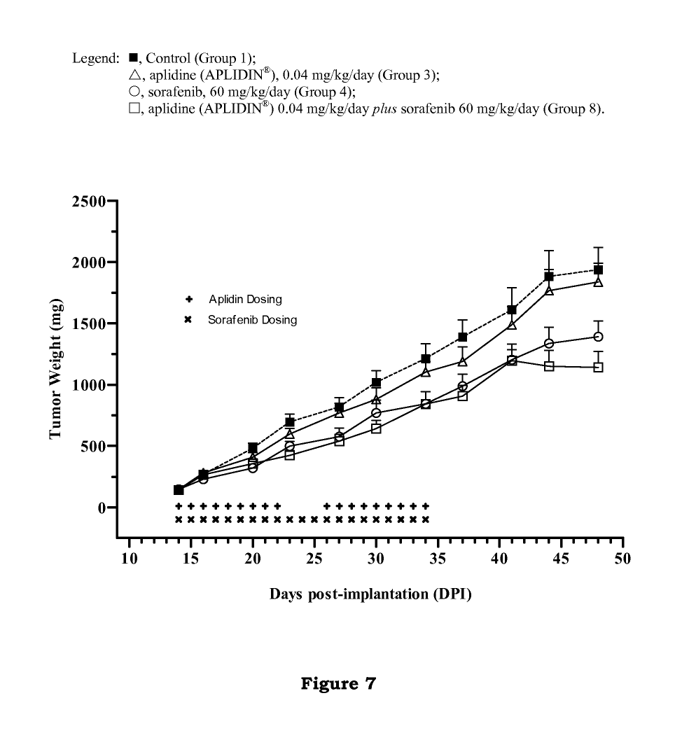

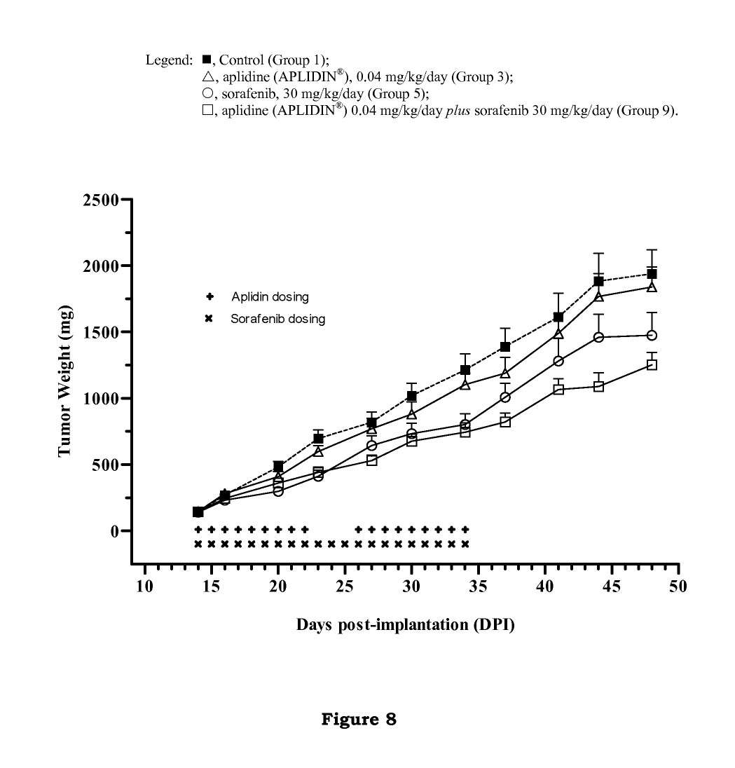

Table VI reports the % T/C values obtained with each of the treatments and FIGS. 5-8 show the tumor weight evolution (mean±SEM) of MRI-H-121 tumors in mice treated with control (vehicle), aplidine, sorafenib, or aplidine plus sorafenib at different doses.

TABLE VI

| | % T/C on day | | | | | | | | | | |
|---|---|---|---|---|---|---|---|---|---|---|---|
| Group | 14 | 16 | 20 | 23 | 27 | 30 | 34 | 37 | 41 | 44 | 48 |
| G1 | — | — | — | — | — | — | — | — | — | — | — |
| (Control | | | | | | | | | | | |
| group) | | | | | | | | | | | |
| G2 | 102.0 | 97.5 | 81.5 | 73.9 | 98.4 | 88.5 | 75.8 | 75.0 | 88.3 | 90.2 | 95.5 |
| G3 | 103.5 | 104.0 | 85.1 | 85.9 | 94.1 | 86.4 | 91.0 | 85.6 | 92.5 | 93.9 | 94.9 |
| G4 | 103.8 | 85.0 | 66.5 | 71.7 | 70.4 | 75.6 | 69.6 | 71.1 | 74.7 | 71.0 | 71.8 |
| G5 | 99.7 | 85.6 | 62.1 | 59.2 | 78.7 | 72.0 | 66.1 | 72.5 | 79.5 | 77.6 | 76.1 |
| G6 | 101.7 | 99.8 | 65.4 | 54.4 | 64.0 | 50.3 | 43.9 | 44.0 | 45.5 | 49.9 | 56.9 |
| G7 | 101.5 | 101.6 | 66.3 | 64.7 | 67.6 | 67.2 | 53.7 | 58.6 | 68.6 | 71.8 | 73.1 |
| G8 | 99.7 | 99.0 | 74.1 | 60.8 | 66.0 | 63.0 | 69.5 | 65.2 | 74.3 | 61.1 | 58.9 |
| G9 | 102.4 | 90.6 | 74.8 | 63.5 | 64.8 | 66.4 | 61.4 | 59.0 | 66.2 | 57.8 | 64.6 |

Table VII shows the % of tumor growth inhibition of aplidine and sorafenib administered as single agents and in combination against MRI-H-121 human renal tumor xenograft at a dose of 0.06 mg/kg/day of aplidine and 60 mg/kg/day of sorafenib. Additionally, the potentiation and the degree of additivity of the combination of aplidine with sorafenib in said doses are provided.

TABLE VII

| | % Inhibition | | | Expected | Actual | Potenti- | Degree of |
|---|---|---|---|---|---|---|---|
| Day | G2 | G4 | G6 | Response | Response | ation | Response |
| 14 | −2.0 | −3.8 | −1.7 | −5.7 | 4.1 | — | — |
| 16 | 2.5 | 15.0 | 0.2 | 17.5 | −17.3 | — | — |
| 20 | 18.5 | 33.5 | 34.6 | 52.1 | −17.5 | no | — |
| 23 | 26.1 | 28.3 | 45.6 | 54.4 | −8.8 | no | — |
| 27 | 1.6 | 29.6 | 36.0 | 31.2 | 4.8 | yes | Additive |
| 30 | 11.5 | 24.4 | 49.7 | 35.9 | 13.8 | yes | Greater |
| | | | | | | | than |
| | | | | | | | additive |
| 34 | 24.2 | 30.4 | 56.1 | 54.6 | 1.4 | yes | Additive |
| 37 | 25.0 | 28.9 | 56.0 | 53.9 | 2.1 | yes | Additive |
| 41 | 11.7 | 25.3 | 54.5 | 37.0 | 17.5 | yes | Greater |
| | | | | | | | than |
| | | | | | | | additive |
| 44 | 9.8 | 29.0 | 50.1 | 38.7 | 11.3 | yes | Greater |
| | | | | | | | than |
| | | | | | | | additive |
| 48 | 4.5 | 28.2 | 43.1 | 32.6 | 10.4 | yes | Greater |
| | | | | | | | than |
| | | | | | | | additive |

Table VIII shows the % of tumor growth inhibition of aplidine and sorafenib administered as single agents and in combination against MRI-H-121 human renal tumor xenograft at a dose of 0.06 mg/kg/day of aplidine and 30 mg/kg/day of sorafenib. Additionally, the potentiation and the degree of additivity of the combination of aplidine with sorafenib in said doses are provided.

TABLE VIII

| | % Inhibition | | | Expected | Actual | Potenti- | Degree of |
|---|---|---|---|---|---|---|---|
| Days | G2 | G5 | G7 | Response | Response | ation | Response |
| 14 | −2.0 | 0.3 | −1.5 | 1.6 | 0.2 | no | |
| 16 | 2.5 | 14.4 | −1.6 | 16.9 | −18.5 | no | |
| 20 | 18.5 | 37.9 | 33.7 | 56.5 | −22.8 | no | |
| 23 | 26.1 | 40.8 | 35.3 | 66.9 | −31.6 | no | |
| 27 | 1.6 | 21.3 | 32.4 | 22.9 | 9.4 | yes | Greater |
| | | | | | | | than |
| | | | | | | | additive |
| 30 | 11.5 | 28.0 | 32.8 | 39.5 | −6.7 | no | — |
| 34 | 24.2 | 33.9 | 46.3 | 58.1 | −11.8 | yes | Less than |
| | | | | | | | additive |
| 37 | 25.0 | 27.5 | 41.4 | 52.6 | −11.2 | yes | Less than |
| | | | | | | | additive |
| 41 | 11.7 | 20.5 | 31.4 | 32.2 | −0.8 | yes | Additive |
| 44 | 9.8 | 22.4 | 28.2 | 32.2 | −4.0 | yes | Additive |
| 48 | 4.5 | 23.9 | 26.9 | 28.3 | −1.4 | no | — |

Table IX shows the % of tumor growth inhibition of aplidine and sorafenib administered as single agents and in combination against MRI-H-121 human renal tumor xenograft at a dose of 0.04 mg/kg/day of aplidine and 60 mg/kg/day of sorafenib. Additionally, the potentiation and the degree of additivity of the combination of aplidine with sorafenib in said doses are provided.

TABLE IX

| | % Inhibition | | | Expected | Actual | Potenti- | Degree of |
|---|---|---|---|---|---|---|---|
| Day | G3 | G4 | G8 | Response | Response | ation | Response |
| 14 | −3.5 | −3.8 | −0.3 | −7.3 | 7.5 | no | — |
| 16 | −4.0 | 15.0 | −1.0 | 11.1 | −10.1 | no | — |
| 20 | 14.9 | 33.5 | −25.9 | 48.5 | −22.5 | no | — |
| 23 | 14.1 | 28.3 | −39.2 | 42.4 | −3.2 | yes | Additive |
| 27 | 5.9 | 29.6 | −34.0 | 35.5 | −1.5 | yes | Additive |
| 30 | 13.6 | 24.4 | −37.0 | 38.0 | −0.9 | yes | Additive |
| 34 | 9.0 | 30.4 | −30.5 | 39.4 | −8.9 | no | — |
| 37 | 14.4 | 28.9 | −34.8 | 43.3 | −8.5 | yes | Less than |
| | | | | | | | additive |
| 41 | 7.5 | 25.3 | −25.7 | 32.9 | −7.2 | no | — |
| 44 | 6.1 | 29.0 | −38.9 | 35.1 | 3.8 | yes | Additive |
| 48 | 5.1 | 28.2 | −41.1 | 33.2 | 7.9 | yes | Greater |
| | | | | | | | than |
| | | | | | | | additive |

Table X shows the % of tumor growth inhibition of aplidine and sorafenib administered as single agents and in combination against MRI-H-121 human renal tumor xenograft at a dose of 0.04 mg/kg/day of aplidine and 30 mg/kg/day of sorafenib. Additionally, the potentiation and the degree of additivity of the combination of aplidine with sorafenib in said doses are provided.

TABLE X

| | % Inhibition | | | Expected | Actual | Potenti- | Degree of |
|---|---|---|---|---|---|---|---|
| Day | G3 | G5 | G9 | Response | Response | ation | Response |
| 14 | −3.5 | 0.3 | −2.4 | −3.2 | 0.8 | — | — |
| 16 | −4.0 | 14.4 | 9.4 | 10.4 | −1.0 | — | — |
| 20 | 14.9 | 37.9 | 25.2 | 52.9 | −27.7 | no | — |
| 23 | 14.1 | 40.8 | 36.5 | 54.8 | −18.3 | no | — |
| 27 | 5.9 | 21.3 | 35.2 | 27.2 | 8.0 | yes | Greater |
| | | | | | | | than |
| | | | | | | | Additive |
| 30 | 13.6 | 28.0 | 33.6 | 41.5 | −8.0 | yes | Less than |
| | | | | | | | Additive |
| 34 | 9.0 | 33.9 | 38.6 | 42.9 | −4.3 | yes | Less than |
| | | | | | | | Additive |
| 37 | 14.4 | 27.5 | 41.0 | 41.9 | −1.0 | yes | Additive |
| 41 | 7.5 | 20.5 | 33.8 | 28.0 | 5.8 | yes | Greater |
| | | | | | | | than |
| | | | | | | | Additive |
| 44 | 6.1 | 22.4 | 42.2 | 28.5 | 13.6 | yes | Greater |
| | | | | | | | than |
| | | | | | | | Additive |
| 48 | 5.1 | 23.9 | 35.4 | 28.9 | 6.4 | yes | Greater |
| | | | | | | | than |
| | | | | | | | Additive |

The MRI-H-121 human renal tumor was refractory to aplidine treatment when administered as single agent (monotherapy) at the doses and schedules tested in this study. In addition, treatment with sorafenib as single agent (monotherapy) administered at 60 or 30 mg/kg/day had a statistical significant inhibition of tumor growth however did not reach the NCI criteria for activity.

On the other hand, when aplidine, at a dose of 0.060 mg/kg/day, was combined with sorafenib, at a dose of 60 mg/kg/day, a statistically significant additive to greater than additive potentiation of antitumor activity was observed. Additionally, the combination of aplidine, at a dose of 0.040 mg/kg/day, with sorafenib, at a dose of 30 mg/kg/day, also resulted in a greater than additive potentiation of antitumor activity.

Finally, the treatments combining aplidine with sorafenib resulted in an acceptable decline in mean body weight, with a maximum of 15% weight loss on day 34. Some isolated individual animals had a body weight loss greater than 20%.

### Results in A498 Human Renal Tumor Xenograft

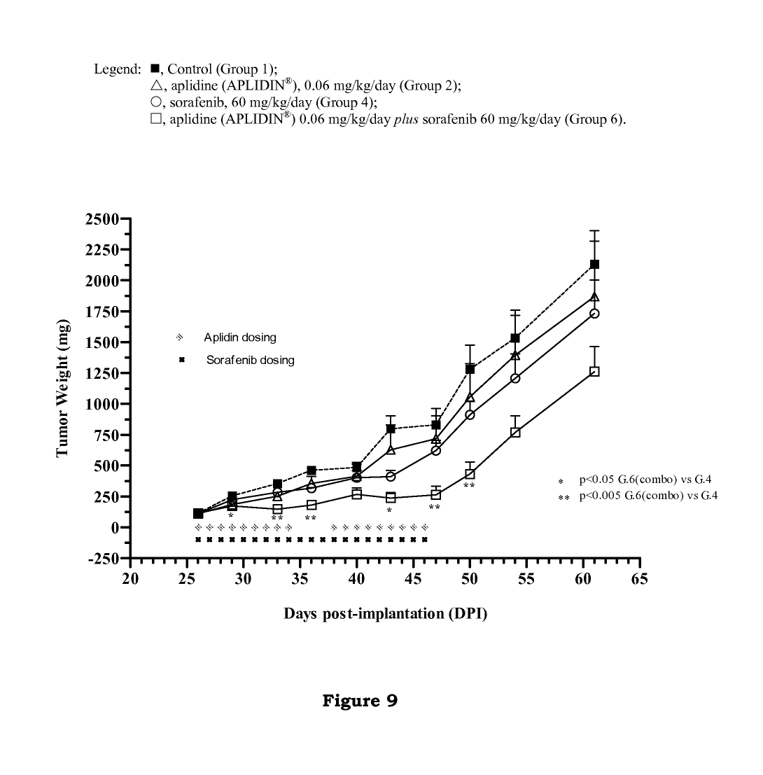

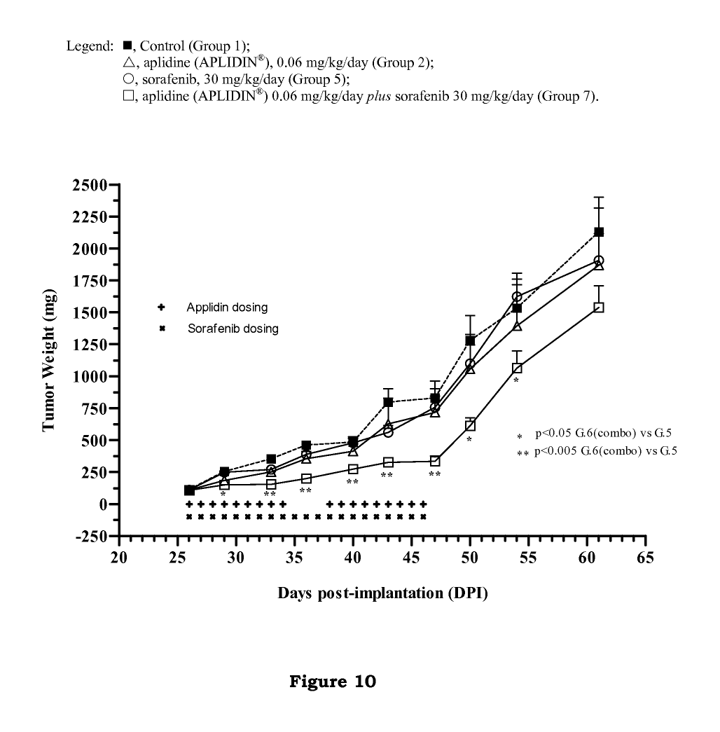

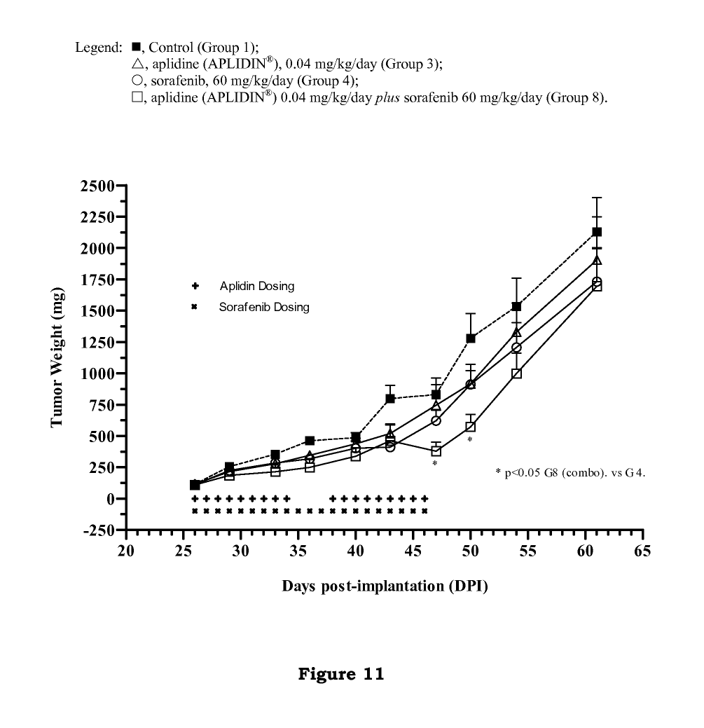

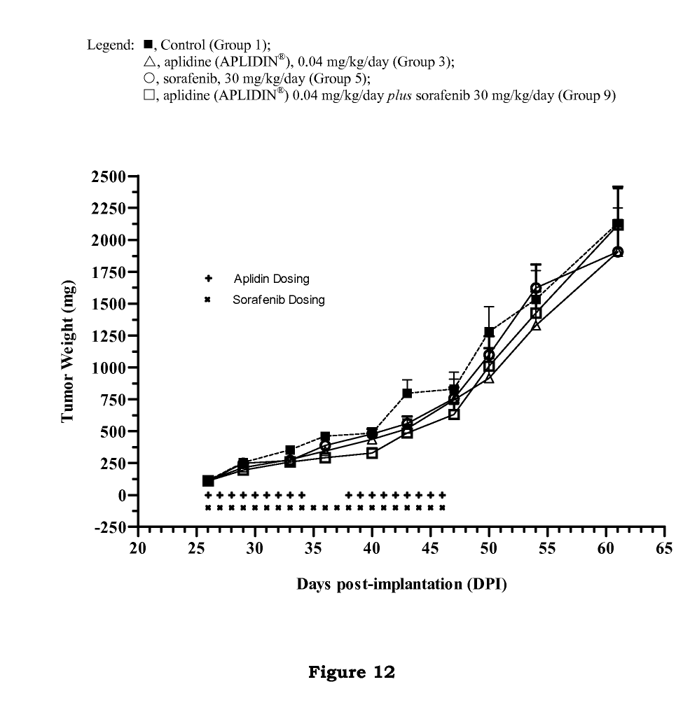

Table XI reports the % T/C values obtained with each of the treatments and FIGS. 9-12 show the tumor weight evolution (mean±SEM) of A498 tumors in mice treated with control (vehicle), aplidine, sorafenib, or aplidine plus sorafenib at different doses.

TABLE XI

| | % T/C on day | | | | | | | | | |
|---|---|---|---|---|---|---|---|---|---|---|
| Group | 26 | 29 | 33 | 36 | 40 | 43 | 47 | 50 | 54 | 61 |
| G1 | — | — | — | — | — | — | — | — | — | — |
| (Control | | | | | | | | | | |
| group) | | | | | | | | | | |
| G2 | 96.8 | 72.3 | 71.0 | 77.0 | 85.3 | 78.6 | 86.5 | 82.6 | 90.9 | 87.8 |
| G3 | 100.8 | 84.6 | 78.7 | 74.8 | 90.2 | 65.1 | 89.8 | 71.5 | 86.7 | 89.5 |
| G4 | 97.4 | 87.8 | 80.2 | 68.6 | 82.9 | 51.4 | 74.9 | 71.1 | 78.7 | 81.3 |
| G5 | 97.0 | 97.4 | 76.6 | 84.2 | 98.7 | 70.1 | 91.2 | 85.9 | 105.9 | 89.6 |
| G6 | 100.7 | 67.8 | 41.3 | 39.1 | 55.0 | 29.6 | 31.7 | 33.7 | 50.1 | 59.2 |
| G7 | 94.1 | 58.6 | 43.7 | 43.2 | 56.5 | 40.8 | 40.4 | 47.9 | 69.4 | 72.3 |
| G8 | 95.3 | 72.3 | 60.6 | 53.9 | 69.6 | 57.8 | 45.7 | 44.7 | 65.1 | 79.7 |
| G9 | 96.8 | 76.2 | 73.1 | 63.5 | 67.7 | 60.9 | 76.2 | 78.9 | 93.0 | 99.4 |

Table XII shows the % of tumor growth inhibition of aplidine and sorafenib administered as single agents and in combination against A498 human renal tumor xenograft at a dose of 0.06 mg/kg/day of aplidine and 60 mg/kg/day of sorafenib. Additionally, the potentiation and the degree of additivity of the combination of aplidine with sorafenib in said doses are provided.

TABLE XII

| | % Inhibition | | | Expected | Actual | Potenti- | Degree of |
|---|---|---|---|---|---|---|---|
| Day | G2 | G4 | G6 | Response | Response | ation | Response |
| 26 | 3.2 | 2.6 | −0.7 | 5.9 | −6.5 | no | — |
| 29 | 27.7 | 12.2 | 32.2 | 40.0 | −7.8 | yes | Less than |
| | | | | | | | additive |
| 33 | 29.0 | 19.8 | 58.7 | 48.8 | 9.9 | yes | Greater |
| | | | | | | | than |
| | | | | | | | additive |
| 36 | 23.0 | 31.4 | 60.9 | 54.4 | 6.5 | yes | Greater |
| | | | | | | | than |
| | | | | | | | additive |
| 40 | 14.7 | 17.1 | 45.0 | 31.8 | 13.2 | yes | Greater |
| | | | | | | | than |
| | | | | | | | additive |
| 43 | 21.4 | 48.5 | 70.4 | 70.0 | 0.4 | yes | Additive |
| 47 | 13.5 | 25.1 | 68.3 | 38.6 | 29.7 | yes | Greater |
| | | | | | | | than |
| | | | | | | | additive |
| 50 | 17.4 | 28.9 | 66.3 | 46.3 | 20.0 | yes | Greater |
| | | | | | | | than |
| | | | | | | | additive |
| 54 | 9.1 | 21.3 | 49.9 | 30.3 | 19.6 | yes | Greater |
| | | | | | | | than |
| | | | | | | | additive |
| 61 | 12.2 | 18.7 | 40.8 | 30.9 | 9.9 | yes | Greater |
| | | | | | | | than |
| | | | | | | | additive |

Table XIII shows the % of tumor growth inhibition of aplidine and sorafenib administered as single agents and in combination against A498 human renal tumor xenograft at a dose of 0.06 mg/kg/day of aplidine and 30 mg/kg/day of sorafenib. Additionally, the potentiation and the degree of additivity of the combination of aplidine with sorafenib in said doses are provided.

TABLE XIII

| | % Inhibition | | | Expected | Actual | Potenti- | Degree of |
|---|---|---|---|---|---|---|---|
| Day | G2 | G5 | G7 | Response | Response | ation | Response |
| 26 | 3.2 | 3.0 | 5.9 | 6.3 | −0.4 | yes | Additive |
| 29 | 27.7 | 2.6 | 41.4 | 30.3 | 11.1 | yes | Greater |
| | | | | | | | than |
| | | | | | | | additive |
| 33 | 29.0 | 23.4 | 56.3 | 52.4 | 3.9 | yes | Greater |
| | | | | | | | than |
| | | | | | | | additive |
| 36 | 23.0 | 15.8 | 56.8 | 38.8 | 18.0 | yes | Greater |
| | | | | | | | than |
| | | | | | | | additive |
| 40 | 14.7 | 1.3 | 43.5 | 16.0 | 27.4 | yes | Greater |
| | | | | | | | than |
| | | | | | | | additive |
| 43 | 21.4 | 29.9 | 59.2 | 51.3 | 7.9 | yes | Greater |
| | | | | | | | than |
| | | | | | | | additive |
| 47 | 13.5 | 8.8 | 59.6 | 22.3 | 37.3 | yes | Greater |
| | | | | | | | than |
| | | | | | | | additive |
| 50 | 17.4 | 14.1 | 52.1 | 31.6 | 20.6 | yes | Greater |
| | | | | | | | than |
| | | | | | | | additive |
| 54 | 9.1 | −5.9 | 30.6 | 3.2 | 27.5 | yes | Greater |
| | | | | | | | than |
| | | | | | | | additive |
| 61 | 12.2 | 10.4 | 27.7 | 22.6 | 5.1 | yes | Greater |
| | | | | | | | than |
| | | | | | | | additive |

Table XIV shows the % of tumor growth inhibition of aplidine and sorafenib administered as single agents and in combination against A498 human renal tumor xenograft at a dose of 0.04 mg/kg/day of aplidine and 60 mg/kg/day of sorafenib. Additionally, the potentiation and the degree of additivity of the combination of aplidine with sorafenib in said doses are provided.

TABLE XIV

| | % Inhibition | | | Expected | Actual | Potenti- | Degree of |
|---|---|---|---|---|---|---|---|
| Day | G3 | G4 | G8 | Response | Response | ation | Response |
| 26 | −0.8 | 2.6 | 4.7 | 1.9 | 2.8 | — | — |
| 29 | 15.4 | 12.2 | 27.7 | 27.7 | 0.0 | yes | Additive |
| 33 | 21.4 | 19.8 | 39.4 | 41.1 | −1.7 | yes | Additive |
| 36 | 25.2 | 31.4 | 46.1 | 56.6 | −10.5 | yes | Less than |
| | | | | | | | additive |
| 40 | 9.8 | 17.1 | 30.4 | 26.9 | 3.4 | yes | Greater |
| | | | | | | | than |
| | | | | | | | additive |
| 43 | 34.9 | 48.5 | 42.2 | 83.5 | −41.3 | no | — |
| 47 | 10.2 | 25.1 | 54.3 | 35.3 | 19.1 | yes | Greater |
| | | | | | | | than |
| | | | | | | | additive |
| 50 | 28.5 | 28.9 | 55.3 | 57.3 | −2.0 | yes | Additive |
| 54 | 13.3 | 21.3 | 34.9 | 34.6 | 0.3 | yes | Additive |
| 61 | 10.5 | 18.7 | 20.3 | 29.1 | −8.8 | yes | Less than |
| | | | | | | | additive |

Table XV shows the % of tumor growth inhibition of aplidine and sorafenib administered as single agents and in combination against A498 human renal tumor xenograft at a dose of 0.04 mg/kg/day of aplidine and 30 mg/kg/day of sorafenib. Additionally, the potentiation and the degree of additivity of the combination of aplidine with sorafenib in said doses are provided.

TABLE XV

| | % Inhibition | | | Expected | Actual | Potenti- | Degree of |
|---|---|---|---|---|---|---|---|
| Day | G3 | G5 | G9 | Response | Response | ation | Response |
| 26 | −0.8 | 3.0 | 3.2 | 2.3 | 0.9 | no | — |
| 29 | 15.4 | 2.6 | 23.8 | 18.0 | 5.8 | no | — |
| 33 | 21.4 | 23.4 | 26.9 | 44.7 | −17.8 | no | — |
| 36 | 25.2 | 15.8 | 36.6 | 41.0 | −4.4 | no | — |
| 40 | 9.8 | 1.3 | 32.3 | 11.2 | 21.1 | yes | Greater |
| | | | | | | | than |
| | | | | | | | additive |
| 43 | 34.9 | 29.9 | 39.1 | 64.8 | −25.8 | no | — |
| 47 | 10.2 | 8.8 | 23.8 | 19.0 | 4.8 | yes | Greater |
| | | | | | | | than |
| | | | | | | | additive |
| 50 | 28.5 | 14.1 | 21.1 | 42.6 | −21.5 | no | — |
| 54 | 13.3 | −5.9 | 7.0 | 7.4 | −0.4 | no | — |
| 61 | 10.5 | 10.4 | 0.6 | 20.9 | −20.3 | no | — |

Treatment of A498 human renal tumor with aplidine or sorafenib administered as single agents (monotherapy), and at the doses and schedules tested in this study, had a trend toward statistical significance against the control group in the inhibition of tumor growth however did not reach the NCI criteria for activity.

A statistically significant antitumor response was observed with the combination groups. A greater than additive potentiation of antitumor activity was observed when aplidine, at a dose of 0.060 mg/kg/day, was combined with sorafenib, at a dose of 60 or 30 mg/kg/day.

Finally, the combination therapy was well tolerated by the mice with no increased evidence of toxicity.

### Example 2

### In Vivo Studies to Determine the Effect of Aplidine in Combination with Sunitinib in Human Renal Tumor Xenografts

The aim of these studies was to evaluate the ability of aplidine to potentiate the antitumoral activity of sunitinib by using three xenograft models of human renal cancer.

Female athymic nude mice (Harlan Sprague Dawley, Madison, Wis.) were utilized for all experiments. Mice were implanted with tumor fragments or a cell suspension when mice were 5 weeks of age. Animals were housed in ventilated rack caging, 5 mice per cage, with food and water ad libitum. The mice were acclimated for 1 week prior to being implanted with tumor fragments or cell suspensions. The Vehicle Control group contained 15 mice and the treated groups had each 10 mice/group.

The tumor models used in these studies were the same as in Example 1 (CAKI-1, MRI-H-121, and A498 cell lines). MRI-H-121 and A498 tumor models were implanted in the animals as disclosed in Example 1.

CAKI-1 cells were grown in McCoy's medium, 2 mM L-glutamine and 10% FBS without antibiotics. Cells from in vitro passage 4-20 were implanted SC into study mice: 5×10⁶ cells/mouse in 0.2 ml 50% Matrigel/50% medium without antibiotics or serum, using a 23 G needle and 1 cc syringe. Bacterial cultures were performed on aliquots of the cells prepared for implantation. All cultures were negative for bacterial contamination at both 24 and 48 hours post-implant.

Tumor measurements were determined as disclosed in Example 1. When tumor growth was within an average size range of 142±52 mg for MRI-H-121, 181±7 mg for CAKI-1, and 207±13 mg for A498 (mean±SD), mice were randomized into the treatment and control groups based on tumor weight by using LabCat® In Life module V 8.0 SP1 tumor tracking and measurement software.

The treatment of MRI-H-121-tumor-bearing mice were initiated on DPI (Day Post Implantation) 10, of CAKI-1 on DPI 23 and of A498 DPI 19. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Sunitinib was provided in the form of a 25 mg capsule containing sunitinib in the form of its malate salt. The formulation was made by solving the capsule content in 0.5% Carboxy Methyl Cellulose (CMC) and further diluting with wfi. The formulation was dosed orally as a suspension to the animals.

Study groups and treatment regimens for the three xenograft models are listed in table XVI.

TABLE XVI

| | N of | | | | Sched- | Test |
|---|---|---|---|---|---|---|
| Group | Animals | Dose | | Route | ule | material |
| G1 | 15 | 10 | ml/kg/day | IP | Qdx9x2 | CEW (0.18/ |
| (Control | | | | | | 0.18/0.84) |
| group) | | 10 | ml/kg/day | PO | Qdx21 | 0.5% CMC |
| G2 | 10 | 0.060 | mg/kg/day | IP | Qdx9x2 | aplidine |
| G3 | 10 | 0.040 | mg/kg/day | IP | Qdx9x2 | aplidine |
| G4 | 10 | 40 | mg/kg/day | PO | Qdx21 | sunitinib |
| G5 | 10 | 30 | mg/kg/day | PO | Qdx21 | sunitinib |
| G6 | 10 | 0.060 | mg/kg/day | IP | Qdx9x2 | aplidine |
| | | 40 | mg/kg/day | PO | Qdx21 | sunitinib |
| G7 | 10 | 0.060 | mg/kg/day | IP | Qdx9x2 | aplidine |
| | | 30 | mg/kg/day | PO | Qdx21 | sunitinib |
| G8 | 10 | 0.040 | mg/kg/day | IP | Qdx9x2 | aplidine |
| | | 40 | mg/kg/day | PO | Qdx21 | sunitinib |
| G9 | 10 | 0.040 | mg/kg/day | IP | Qdx9x2 | aplidine |
| | | 30 | mg/kg/day | PO | Qdx21 | sunitinib |

IP: Intraperitoneal administration; PO: Oral administration
Qdx9x2: Two cycles wherein test material is administered every day for 9 consecutive days with a rest period of 4-5 days between cycles
Qdx21: Administration of the test material every day for 21 consecutive days

Tumor size measurements were recorded twice weekly from the treatment initiation until the termination of the study. Tumor growth inhibition was assessed comparing the mean tumor weight between the two agents in combination (aplidine and sunitinib) against sunitinib mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 1.

Body weight effects were determined by comparing each mouse body weight measurement with the initial body weight (first day of treatment). The NCI activity criterion of body weight loss >20% was used to gauge compound toxicity.

### Results in CAKI-1 Human Renal Tumor Xenograft

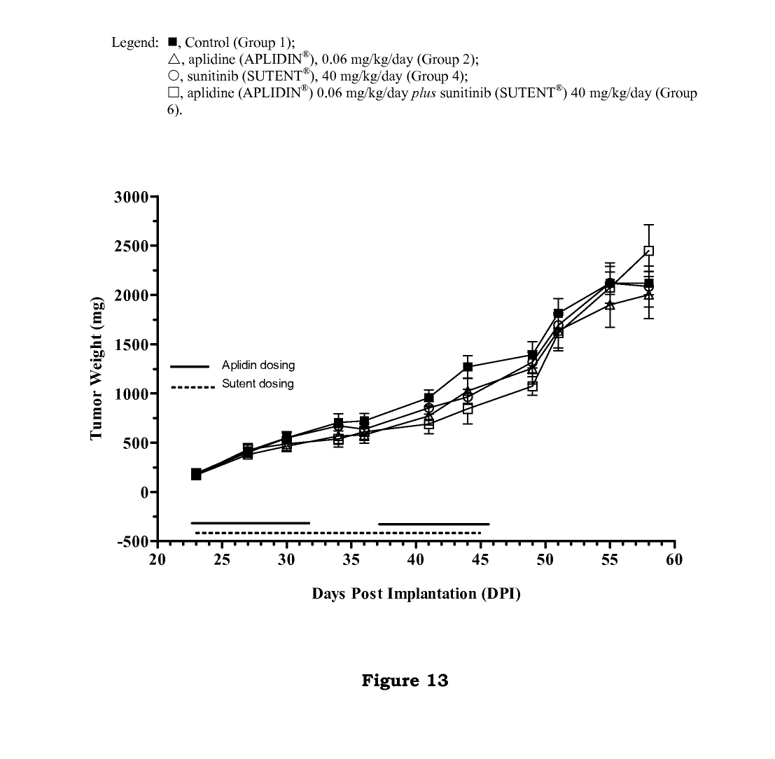

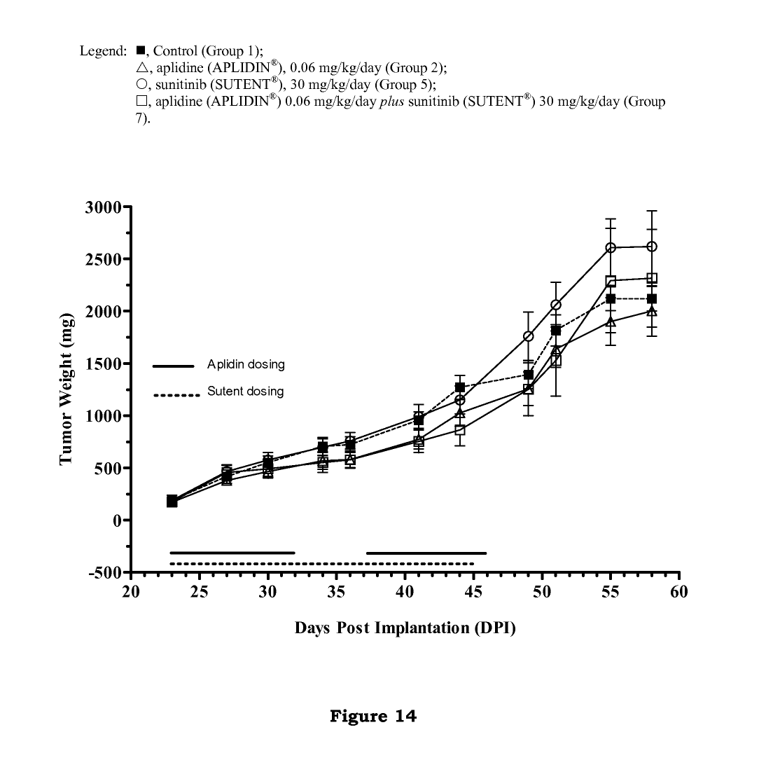

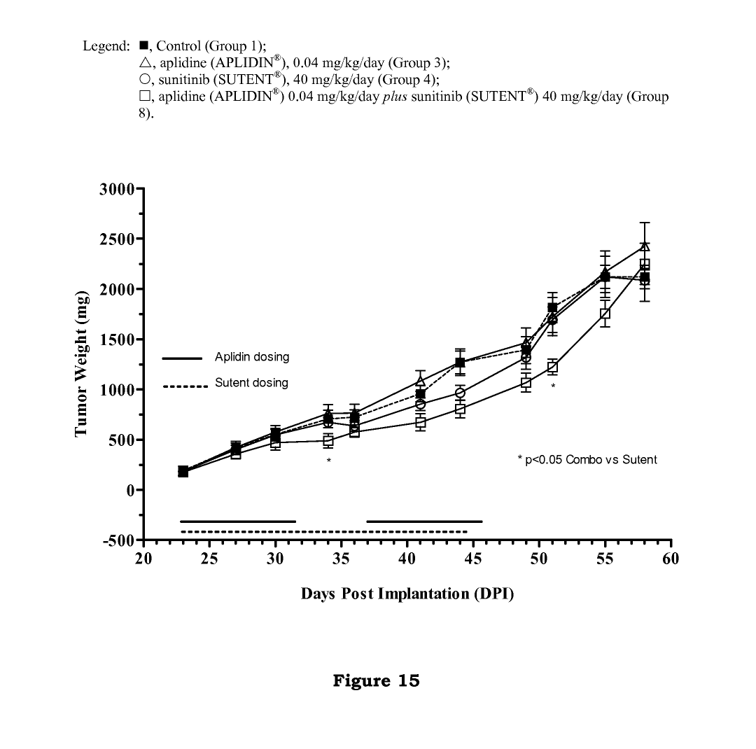

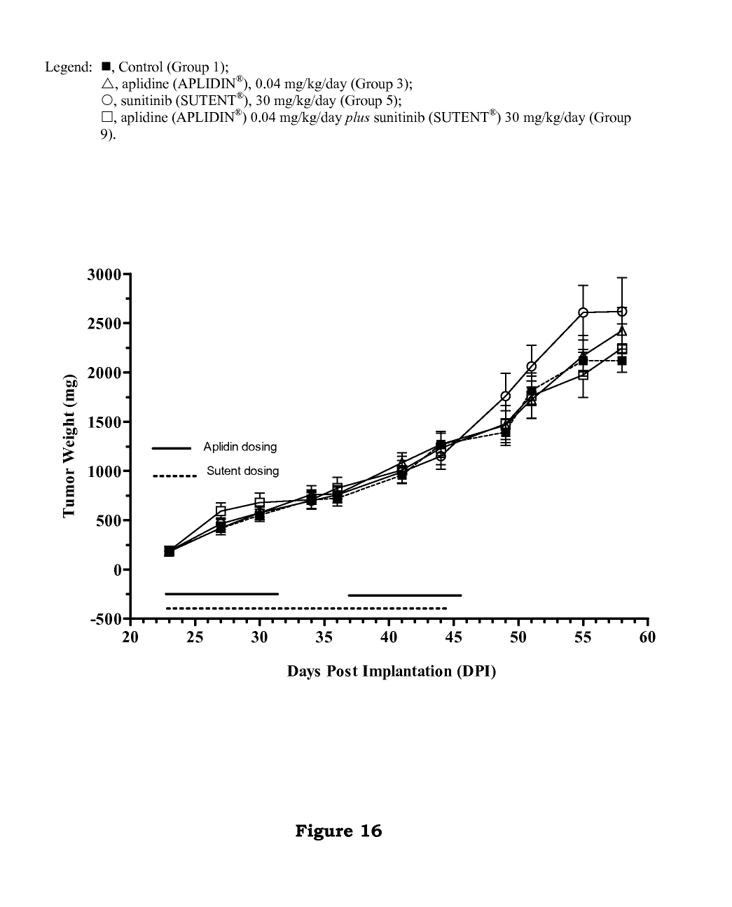

Table XVII reports the % T/C values obtained with each of the treatments and FIGS. 13-16 show the tumor weight evolution (mean±SEM) of CAKI-1 tumors in mice treated with control (vehicle), aplidine, sunitinib, or aplidine plus sunitinib at different doses.

TABLE XVII

| | % T/C on day | | | | | |
|---|---|---|---|---|---|---|
| Group | 23 | 27 | 30 | 34 | 36 | 41 |
| G1 | — | — | — | — | — | — |
| (Control | | | | | | |
| group) | | | | | | |
| G2 | 90.33 | 90.94 | 84.62 | 80.57 | 80.30 | 80.79 |
| G3 | 94.62 | 101.52 | 104.62 | 107.71 | 105.76 | 113.26 |
| G4 | 95.88 | 96.41 | 99.68 | 95.13 | 88.14 | 89.22 |
| G5 | 98.81 | 111.52 | 104.73 | 98.93 | 104.92 | 103.65 |
| G6 | 92.51 | 103.62 | 88.88 | 76.18 | 84.56 | 72.27 |
| G7 | 92.76 | 109.48 | 89.10 | 78.16 | 79.99 | 78.70 |
| G8 | 91.71 | 85.85 | 85.41 | 69.05 | 79.64 | 70.35 |
| G9 | 99.92 | 142.26 | 123.52 | 100.40 | 114.34 | 105.44 |

| | % T/C on day | | | | |
|---|---|---|---|---|---|
| Group | 44 | 49 | 51 | 55 | 58 |
| G1 | — | — | — | — | — |
| (Control | | | | | |
| group) | | | | | |
| G2 | 80.84 | 90.24 | 90.18 | 89.69 | 94.45 |
| G3 | 100.00 | 105.27 | 94.96 | 102.37 | 114.40 |
| G4 | 76.01 | 94.67 | 93.33 | 100.06 | 98.39 |
| G5 | 90.62 | 126.46 | 113.64 | 123.04 | 123.58 |
| G6 | 66.52 | 77.20 | 88.95 | 97.88 | 115.53 |
| G7 | 67.98 | 89.96 | 84.21 | 108.22 | 109.25 |
| G8 | 63.46 | 76.68 | 67.39 | 82.79 | 106.19 |
| G9 | 96.97 | 106.15 | 97.20 | 93.20 | 105.99 |

Table XVIII shows the % of tumor growth inhibition of aplidine and sunitinib administered as single agents and in combination against CAKI-1 human renal tumor xenograft at a dose of 0.06 mg/kg/day of aplidine and 30 mg/kg/day of sunitinib. Additionally, the potentiation and the degree of additivity of the combination of aplidine with sunitinib at said doses are provided.

TABLE XVIII

| | % Inhibition | | | Expected | Actual | Potent- | Degree of |
|---|---|---|---|---|---|---|---|
| Day | G2 | G5 | G7 | Response | Response | ation | Response |
| 23 | 9.7 | 1.2 | 7.2 | 10.9 | −3.6 | no | — |
| 27 | 9.1 | −11.5 | −9.5 | −2.5 | −7.0 | no | — |
| 30 | 15.4 | −4.7 | 10.9 | 10.6 | 0.3 | no | — |
| 34 | 19.4 | 1.1 | 21.8 | 20.5 | 1.3 | no | — |
| 36 | 19.7 | 4.9 | 20.0 | 14.8 | 5.2 | no | — |
| 41 | 19.2 | −3.7 | 21.3 | 15.6 | 5.7 | no | — |
| 44 | 19.2 | 9.4 | 32.0 | 28.5 | 3.5 | no | — |
| 49 | 9.8 | −26.5 | 10.0 | −16.7 | 26.7 | yes | greater |
| | | | | | | | than |
| | | | | | | | additive |
| 51 | 9.8 | −13.6 | 15.8 | −3.8 | 19.6 | yes | — |
| 55 | 10.3 | 23.0 | −8.2 | −12.7 | 4.5 | yes | — |
| 58 | 5.6 | −23.6 | −9.2 | −18.0 | 8.8 | yes | — |

Table XIX shows the % of tumor growth inhibition of aplidine and sunitinib administered as single agents and in combination against CAKI-1 human renal tumor xenograft at a dose of 0.04 mg/kg/day of aplidine and 40 mg/kg/day of sunitinib. Additionally, the potentiation and the degree of additivity of the combination of aplidine with sunitinib at said doses are provided.

TABLE XIX

| | % Inhibition | | | Expected | Actual | Potent- | Degree of |
|---|---|---|---|---|---|---|---|
| Day | G3 | G4 | G8 | Response | Response | ation | Response |
| 23 | 5.4 | 4.1 | 8.3 | 9.5 | −1.2 | no | — |
| 27 | −1.5 | 3.6 | 14.1 | 2.1 | 12.1 | yes | Greater |
| | | | | | | | than |
| | | | | | | | additive |
| 30 | −4.6 | 0.3 | 14.6 | −4.3 | 18.9 | yes | Greater |
| | | | | | | | than |
| | | | | | | | additive |
| 34 | −7.7 | 4.9 | 31.0 | −2.8 | 33.8 | yes | Greater |
| | | | | | | | than |
| | | | | | | | additive |
| 36 | 5.8 | 11.9 | 20.4 | 6.1 | 14.3 | yes | Greater |
| | | | | | | | than |
| | | | | | | | additive |
| 41 | −13.3 | 10.8 | 29.7 | −2.5 | 32.1 | yes | Greater |
| | | | | | | | than |
| | | | | | | | additive |
| 44 | 0.0 | 24.0 | 36.5 | 24.0 | 12.6 | yes | Greater |
| | | | | | | | than |
| | | | | | | | additive |
| 49 | −5.3 | 5.3 | 23.3 | 0.1 | 23.3 | yes | Greater |
| | | | | | | | than |
| | | | | | | | additive |
| 51 | 5.0 | 6.7 | 32.6 | 11.7 | 20.9 | yes | Greater |
| | | | | | | | than |
| | | | | | | | additive |
| 55 | −2.4 | −0.1 | 17.2 | −2.4 | 19.6 | yes | Greater |
| | | | | | | | than |
| | | | | | | | additive |
| 58 | −14.4 | 1.6 | −6.2 | −12.8 | 6.6 | no | — |

Table XX shows the % of tumor growth inhibition of aplidine and sunitinib administered as single agents and in combination against CAKI-1 human renal tumor xenograft at a dose of 0.04 mg/kg/day of aplidine and 30 mg/kg/day of sunitinib. Additionally, the potentiation and the degree of additivity of the combination of aplidine with sunitinib at said doses are provided.

TABLE XX

| | % Inhibition | | | Expected | Actual | Potent- | Degree of |
|---|---|---|---|---|---|---|---|
| Day | G3 | G5 | G9 | Response | Response | ation | Response |
| 23 | 5.4 | 1.2 | 0.1 | 6.6 | −6.5 | no | — |
| 27 | −1.5 | −11.5 | −42.3 | −13.0 | −29.2 | no | — |
| 30 | −4.6 | −4.7 | −23.5 | −9.4 | −14.2 | no | — |
| 34 | −7.7 | 1.1 | −0.4 | −6.6 | 6.2 | no | — |
| 36 | 5.8 | 4.9 | −14.3 | −10.7 | −3.7 | no | — |
| 41 | −13.3 | −3.7 | −5.4 | −16.9 | 11.5 | no | — |
| 44 | 0.0 | 9.4 | 3.0 | 9.4 | −6.4 | no | — |
| 49 | −5.3 | −26.5 | −6.1 | −31.7 | 25.6 | no | — |
| 51 | 5.0 | −13.6 | 2.8 | −8.6 | 11.4 | no | — |
| 55 | −2.4 | 23.0 | 6.8 | −25.4 | 32.2 | yes | Greater |
| | | | | | | | than |
| | | | | | | | additive |
| 58 | −14.4 | −23.6 | −6.0 | −38.0 | 32.0 | no | — |

When aplidine and sunitinib were administered as single agents (monotherapy) against CAKI-1 human renal tumor cell line, they were inactive. Additionally, these treatments did not cause a significant decline in mean body weight, and all mice gained weight by the end of the study.

However, when aplidine, at a dose of 0.040 mg/kg/day, was combined with sunitinib at a dose of 40 mg/kg/day, a potentiation of activity was observed, resulting in a greater than additive potentiation of tumor growth inhibition. In addition, the combination of aplidine with sunitinib caused an acceptable decline in body weight, with a maximum of 7.2% weight loss.

### Results in MRI-H-121 Human Renal Tumor Xenograft

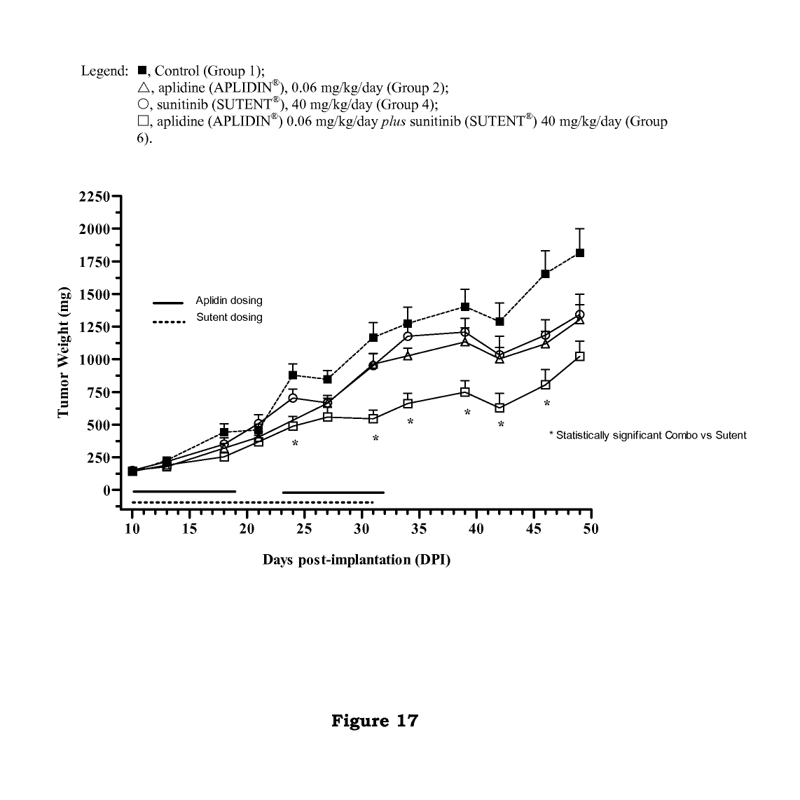

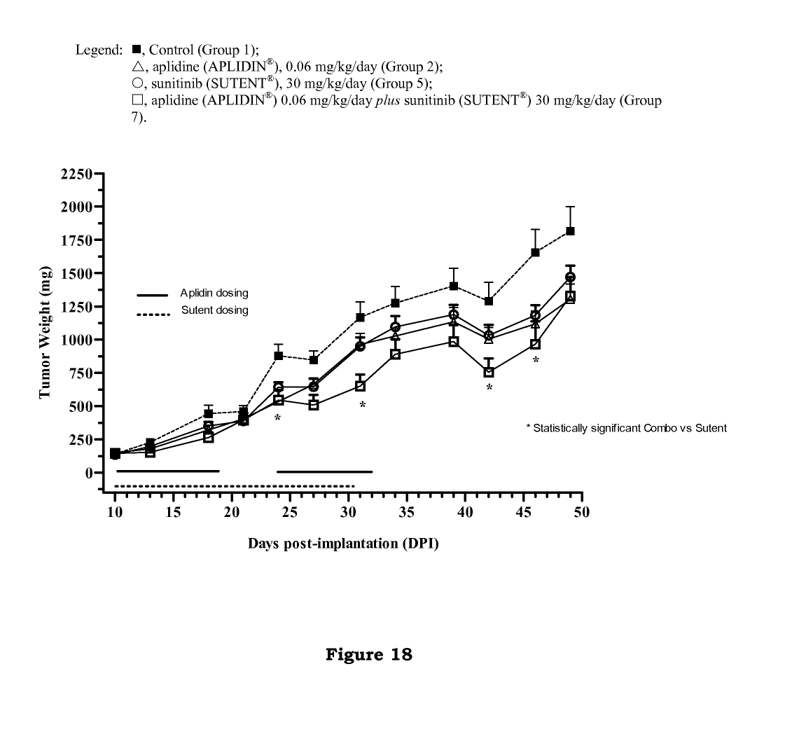

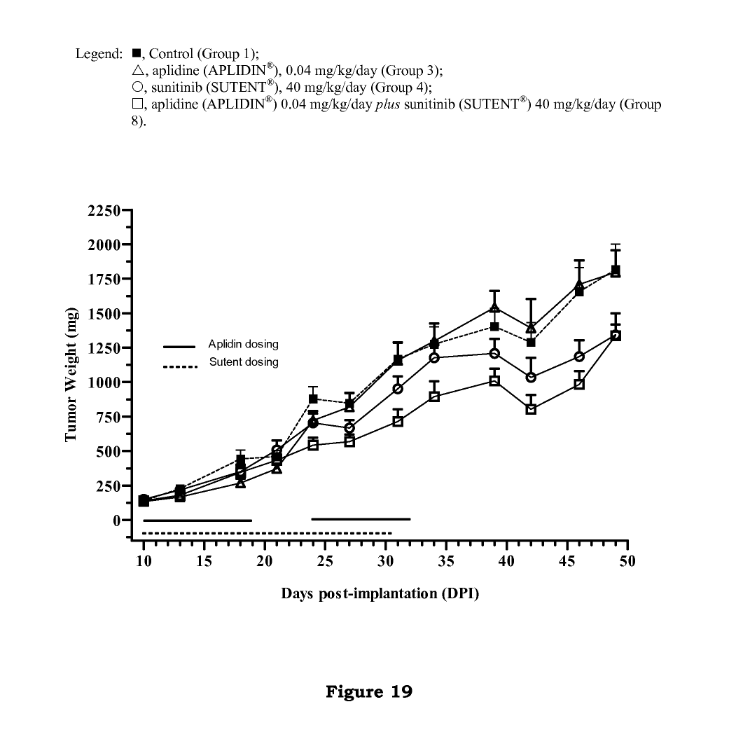

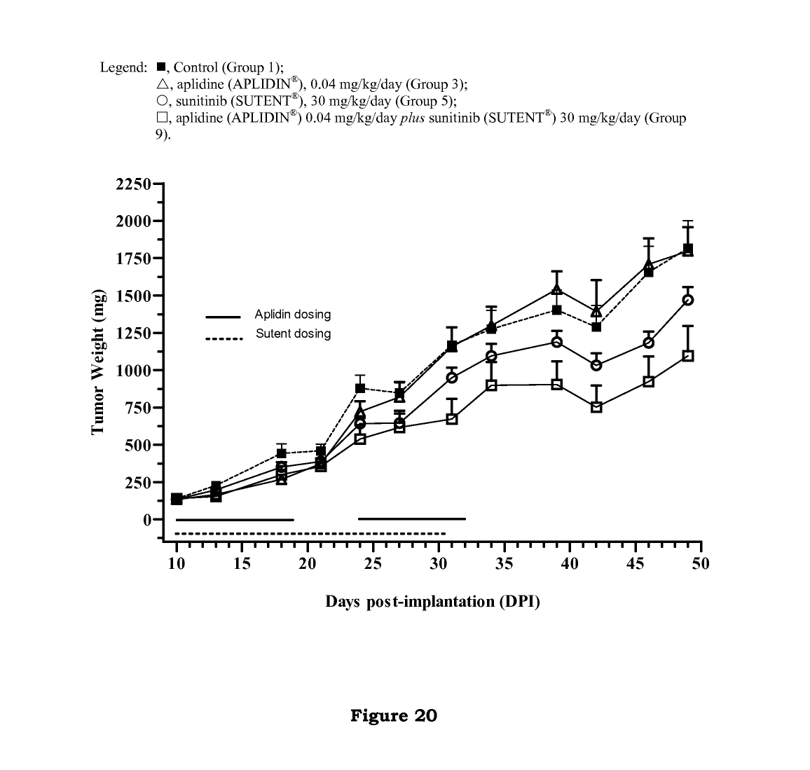

Table XXI reports the % T/C values obtained with each of the treatments and FIGS. 17-20 show the tumor weight evolution (mean±SEM) of MRI-H-121 tumors in mice treated with control (vehicle), aplidine, sunitinib, or aplidine plus sunitinib at different doses.

TABLE XXI

| | % T/C on day | | | | |
|---|---|---|---|---|---|
| Group | 10 | 13 | 18 | 21 | 27 |
| G1 | — | — | — | — | — |
| (Control | | | | | |
| group) | | | | | |
| G2 | 108.70 | 79.07 | 72.53 | 87.99 | 78.35 |
| G3 | 96.38 | 73.72 | 60.59 | 81.08 | 96.89 |
| G4 | 108.36 | 96.29 | 79.33 | 110.64 | 78.89 |
| G5 | 97.20 | 87.21 | 79.17 | 84.52 | 76.30 |
| G6 | 103.12 | 84.48 | 57.61 | 80.35 | 65.88 |
| G7 | 105.09 | 67.70 | 59.59 | 86.23 | 60.13 |
| G8 | 100.54 | 79.52 | 78.22 | 94.45 | 67.13 |
| G9 | 100.97 | 68.40 | 67.92 | 77.57 | 72.92 |

| | % T/C on day | | | | | |
|---|---|---|---|---|---|---|
| Group | 31 | 34 | 39 | 42 | 46 | 49 |
| G1 | — | — | — | — | — | — |
| (Control | | | | | | |
| group) | | | | | | |
| G2 | 82.53 | 80.60 | 80.78 | 77.98 | 67.60 | 71.83 |
| G3 | 99.23 | 101.86 | 109.79 | 108.15 | 103.30 | 98.97 |
| G4 | 81.59 | 92.32 | 86.16 | 80.27 | 71.65 | 73.97 |
| G5 | 81.39 | 88.33 | 84.63 | 80.17 | 71.53 | 80.97 |
| G6 | 46.71 | 51.94 | 53.36 | 48.74 | 48.63 | 56.36 |
| G7 | 55.79 | 69.82 | 70.18 | 58.58 | 58.38 | 73.04 |
| G8 | 61.22 | 70.24 | 71.90 | 62.33 | 59.43 | 73.67 |
| G9 | 57.67 | 70.52 | 64.40 | 58.47 | 55.79 | 60.32 |

Table XXII shows the % of tumor growth inhibition of aplidine and sunitinib administered as single agents and in combination against MRI-H-121 human renal tumor xenograft at a dose of 0.06 mg/kg/day of aplidine and 40 mg/kg/day of sunitinib. Additionally, the potentiation and the degree of additivity of the combination of aplidine with sunitinib at said doses are provided.

TABLE XXII

| | % Inhibition | | | Expected | Actual | Potent- | Degree of |
|---|---|---|---|---|---|---|---|
| Day | G2 | G4 | G6 | Response | Response | ation | Response |
| 10 | −8.7 | −8.4 | −3.1 | −17.1 | 13.9 | no | — |
| 13 | 20.9 | 3.7 | 15.5 | 24.6 | −9.1 | no | — |
| 18 | 27.5 | 20.7 | 42.4 | 48.1 | −5.8 | no | — |
| 21 | 12.0 | −10.6 | 19.7 | 1.4 | 18.3 | no | — |
| 27 | 21.6 | 21.1 | 34.1 | 42.8 | −8.6 | yes | Less than |
| | | | | | | | additive |
| 31 | 17.5 | 18.4 | 53.3 | 35.9 | 17.4 | yes | Greater |
| | | | | | | | than |
| | | | | | | | additive |
| 34 | 19.4 | 7.7 | 48.1 | 27.1 | 21.0 | yes | Greater |
| | | | | | | | than |
| | | | | | | | additive |
| 39 | 19.2 | 13.8 | 46.6 | 33.1 | 13.6 | yes | Greater |
| | | | | | | | than |
| | | | | | | | additive |
| 42 | 22.0 | 19.7 | 51.3 | 41.7 | 9.5 | yes | Greater |
| | | | | | | | than |
| | | | | | | | additive |
| 46 | 32.4 | 28.4 | 51.4 | 60.8 | −9.4 | yes | Less than |
| | | | | | | | additive |
| 49 | 28.2 | 26.0 | 43.6 | 54.2 | −10.6 | no | — |

Table XXIII shows the % of tumor growth inhibition of aplidine and sunitinib administered as single agents and in combination against MRI-H-121 human renal tumor xenograft at a dose of 0.06 mg/kg/day of aplidine and 30 mg/kg/day of sunitinib. Additionally, the potentiation and the degree of additivity of the combination of aplidine with sunitinib at said doses are provided.

TABLE XXIII

| | % Inhibition | | | Expected | Actual | Potent- | Degree of |
|---|---|---|---|---|---|---|---|
| Day | G2 | G5 | G7 | Response | Response | ation | Response |
| 10 | −8.7 | 2.8 | −5.1 | −5.9 | 0.8 | no | — |
| 13 | 20.9 | 12.8 | 32.3 | 33.7 | −1.4 | no | — |
| 18 | 27.5 | 20.8 | 40.4 | 48.3 | −7.9 | no | — |
| 21 | 12.0 | 15.5 | 13.8 | 27.5 | −13.7 | no | — |
| 27 | 21.6 | 23.7 | 39.9 | 45.3 | −5.5 | yes | Less than |
| | | | | | | | additive |
| 31 | 17.5 | 18.6 | 44.2 | 36.1 | 8.1 | yes | Greater |
| | | | | | | | than |
| | | | | | | | additive |
| 34 | 19.4 | 11.7 | 30.2 | 31.1 | −0.9 | yes | Less than |
| | | | | | | | additive |
| 39 | 19.2 | 15.4 | 29.8 | 34.6 | −4.8 | yes | Less than |
| | | | | | | | additive |
| 42 | 22.0 | 19.8 | 41.4 | 41.8 | −0.4 | yes | Less than |
| | | | | | | | additive |
| 46 | 32.4 | 28.5 | 41.6 | 60.9 | −19.3 | yes | Less than |
| | | | | | | | additive |
| 49 | 28.2 | 19.0 | 27.0 | 47.2 | −20.2 | no | — |

Table XXIV shows the % of tumor growth inhibition of aplidine and sunitinib administered as single agents and in combination against MRI-H-121 human renal tumor xenograft at a dose of 0.04 mg/kg/day of aplidine and 40 mg/kg/day of sunitinib. Additionally, the potentiation and the degree of additivity of the combination of aplidine with sunitinib at said doses are provided.

TABLE XXIV

| | % Inhibition | | | Expected | Actual | Potent- | Degree of |
|---|---|---|---|---|---|---|---|
| Day | G3 | G4 | G8 | Response | Response | ation | Response |
| 10 | 3.6 | −8.4 | −0.5 | −4.7 | 4.2 | no | — |
| 13 | 26.3 | 3.7 | 20.5 | 30.0 | −9.5 | no | — |
| 18 | 39.4 | 20.7 | 21.8 | 60.1 | −38.3 | no | — |
| 21 | 18.9 | −10.6 | 5.5 | 8.3 | −2.7 | no | — |
| 27 | 3.1 | 21.1 | 32.9 | 24.2 | 8.7 | yes | Greater |
| | | | | | | | than |
| | | | | | | | additive |
| 31 | 0.8 | 18.4 | 38.8 | 19.2 | 19.6 | yes | Greater |
| | | | | | | | than |
| | | | | | | | additive |
| 34 | −1.9 | 7.7 | 29.8 | 5.8 | 23.9 | yes | Greater |
| | | | | | | | than |
| | | | | | | | additive |
| 39 | −9.8 | 13.8 | 28.1 | 4.0 | 24.1 | yes | Greater |
| | | | | | | | than |
| | | | | | | | additive |
| 42 | −8.2 | 19.7 | 37.7 | 11.6 | 26.1 | yes | Greater |
| | | | | | | | than |
| | | | | | | | additive |
| 46 | −3.3 | 28.4 | 40.6 | 25.1 | 15.5 | yes | Greater |
| | | | | | | | than |
| | | | | | | | additive |
| 49 | 1.0 | 26.0 | 26.3 | 27.1 | 0.7 | no | — |

Table XXV shows the % of tumor growth inhibition of aplidine and sunitinib administered as single agents and in combination against MRI-H-121 human renal tumor xenograft at a dose of 0.04 mg/kg/day of aplidine and 30 mg/kg/day of sunitinib. Additionally, the potentiation and the degree of additivity of the combination of aplidine with sunitinib at said doses are provided.

TABLE XXV

| | % Inhibition | | | Expected | Actual | Potent- | Degree of |
|---|---|---|---|---|---|---|---|
| Day | G3 | G5 | G9 | Response | Response | ation | Response |
| 10 | 3.6 | 2.8 | −4.1 | 6.4 | −10.5 | no | — |
| 13 | 26.3 | 12.8 | 26.0 | 39.1 | −13.1 | no | — |
| 18 | 39.4 | 20.8 | 25.1 | 60.2 | −35.2 | yes | Less than |
| | | | | | | | additive |
| 21 | 18.9 | 15.5 | 13.9 | 34.4 | −20.5 | no | — |
| 27 | 3.1 | 23.7 | 19.0 | 26.8 | −7.8 | yes | Less than |
| | | | | | | | additive |
| 31 | 0.8 | 18.6 | 35.9 | 19.4 | 16.5 | yes | Greater |
| | | | | | | | than |
| | | | | | | | additive |
| 34 | −1.9 | 11.7 | 21.6 | 9.8 | 11.8 | yes | Greater |
| | | | | | | | than |
| | | | | | | | additive |
| 39 | −9.8 | 15.4 | 28.4 | 5.6 | 22.9 | yes | Greater |
| | | | | | | | than |
| | | | | | | | additive |
| 42 | −8.2 | 19.8 | 27.1 | 11.7 | 15.4 | yes | Greater |
| | | | | | | | than |
| | | | | | | | additive |
| 46 | −3.3 | 28.5 | 30.5 | 25.2 | 5.3 | yes | Additive |
| 49 | 1.0 | 19.0 | 24.6 | 20.1 | 4.5 | no | — |

The MRI-H-121 human renal tumor was refractory to aplidine and sunitinib when administered as single agents (monotherapy) in the doses and schedules tested, although aplidine treatment showed hints of activity on inhibition of tumor growth at 0.060 mg/kg/day. Additionally, these treatments did not cause a significant decline in mean body weight, and all mice gained weight by the end of the study.

However, when both drugs were combined, a statistically significant potentiation of activity was observed at all dose levels tested. This potentiation was determined to be greater than additive. In addition, the combination of aplidine with sunitinib caused an acceptable decline in body weight, with a maximum of 8.7% weight loss.

### Results in A498 Human Renal Tumor Xenograft

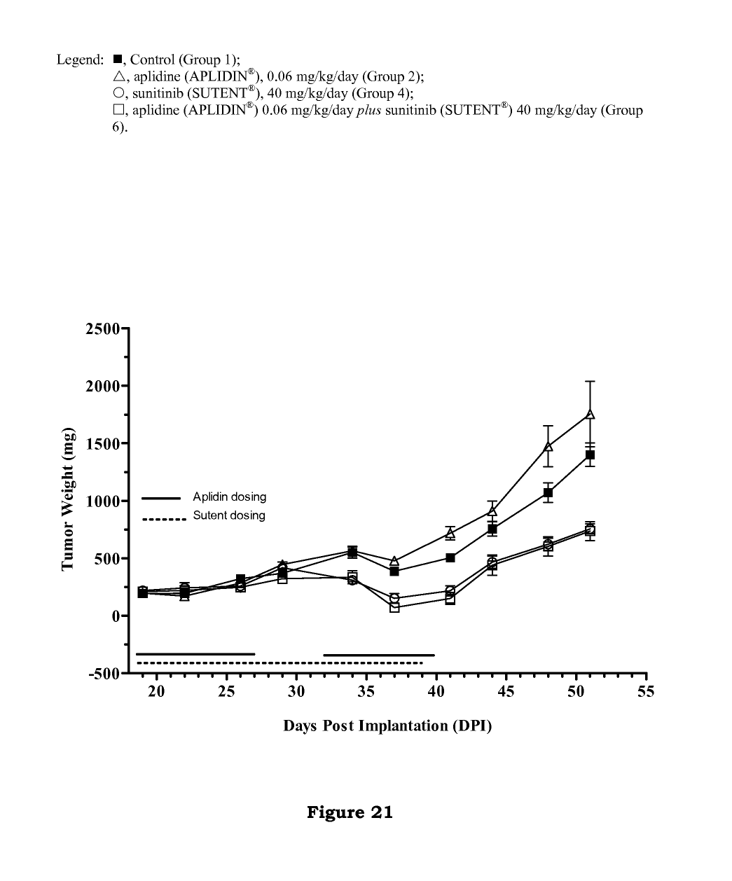

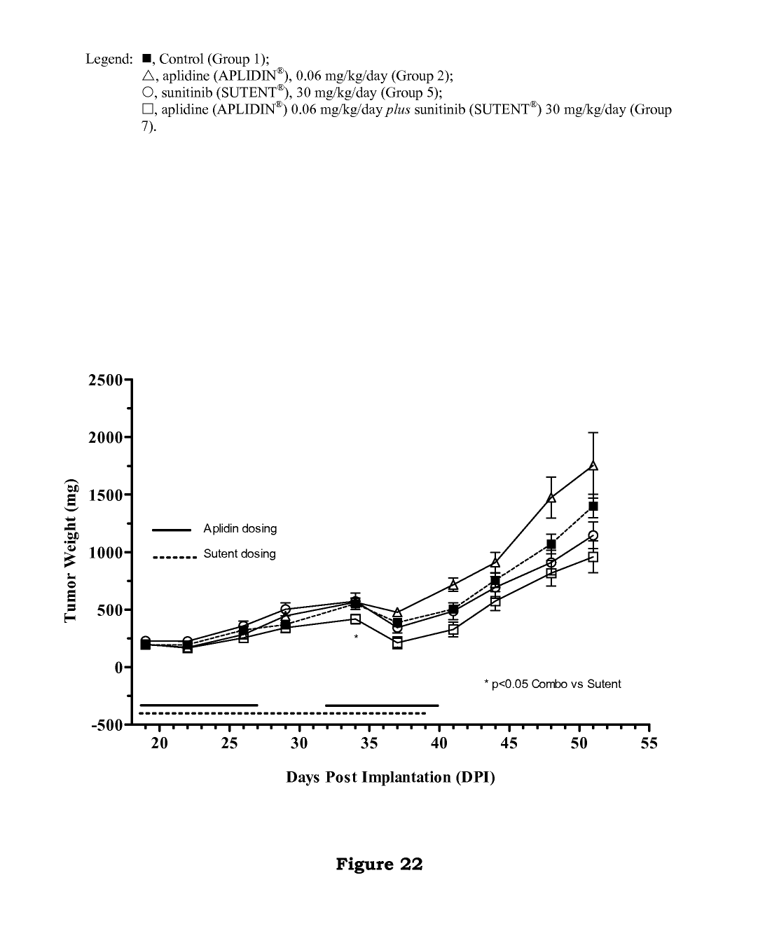

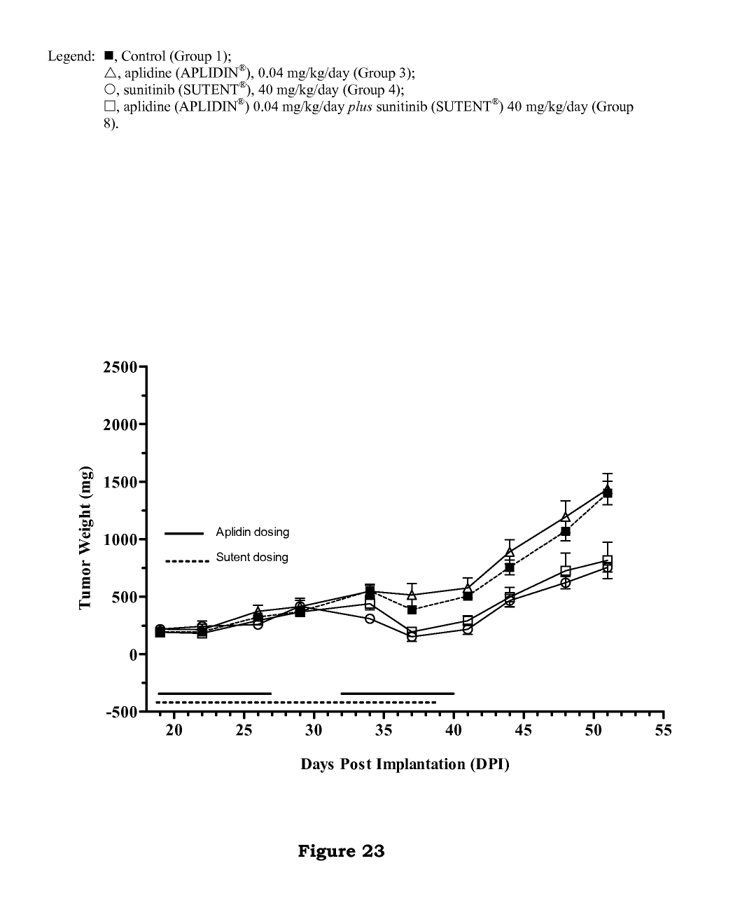

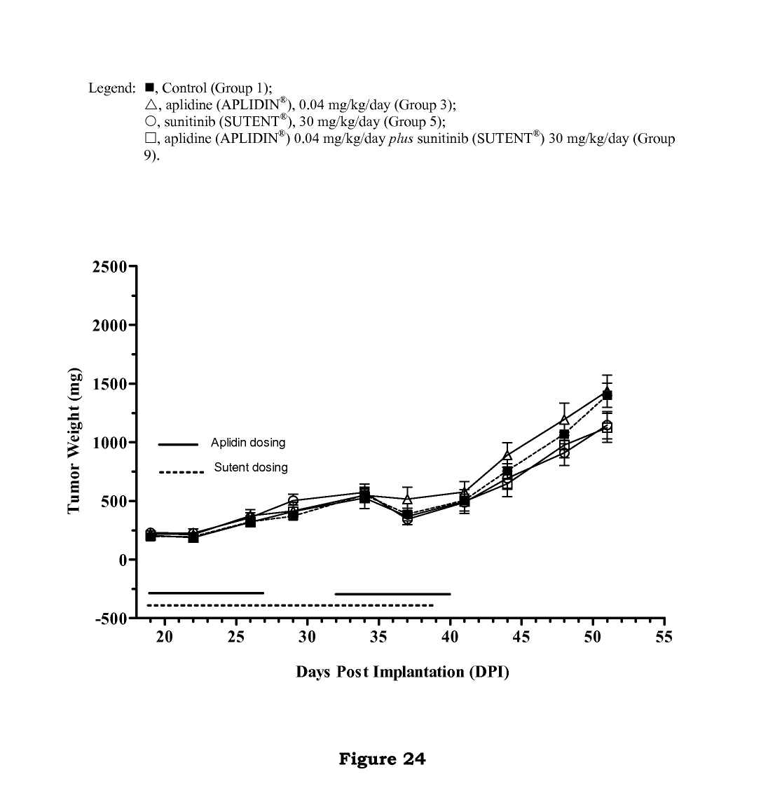

Table XXVI reports the % T/C values obtained with each of the treatments and FIGS. 21-24 show the tumor weight evolution (mean±SEM) of A498 tumors in mice treated with control (vehicle), aplidine, sunitinib, or aplidine plus sunitinib at different doses.

TABLE XXVI

| | % T/C on day | | | | | | | | | |
|---|---|---|---|---|---|---|---|---|---|---|
| Group | 19 | 22 | 26 | 29 | 34 | 37 | 41 | 44 | 48 | 51 |
| G1 | — | — | — | — | — | — | — | — | — | — |
| (Control | | | | | | | | | | |
| group) | | | | | | | | | | |
| G2 | 101.0 | 87.5 | 88.3 | 120.5 | 102.7 | 123.5 | 142.2 | 120.5 | 137.8 | 125.2 |
| G3 | 113.0 | 109.5 | 116.0 | 111.9 | 99.4 | 133.2 | 114.2 | 118.1 | 111.6 | 102.5 |
| G4 | 112.8 | 124.9 | 80.5 | 112.8 | 56.1 | 39.3 | 43.1 | 61.7 | 58.1 | 54.0 |
| G5 | 117.3 | 115.8 | 111.6 | 136.0 | 104.0 | 89.0 | 96.5 | 91.9 | 84.9 | 81.8 |
| G6 | 108.0 | 112.8 | 77.1 | 87.2 | 60.9 | 18.3 | 29.9 | 58.2 | 56.4 | 52.6 |
| G7 | 102.9 | 85.3 | 79.2 | 92.7 | 76.0 | 55.0 | 65.0 | 76.1 | 76.3 | 68.5 |
| G8 | 99.2 | 93.7 | 91.2 | 99.5 | 79.9 | 50.4 | 57.8 | 65.9 | 67.8 | 58.2 |
| G9 | 106.5 | 96.3 | 99.3 | 110.7 | 95.3 | 95.2 | 98.1 | 85.6 | 91.2 | 80.2 |

Table XXVII shows the % of tumor growth inhibition of aplidine and sunitinib administered as single agents and in combination against A498 human renal tumor xenograft at a dose of 0.06 mg/kg/day of aplidine and 40 mg/kg/day of sunitinib. Additionally, the potentiation and the degree of additivity of the combination of aplidine with sunitinib at said doses are provided.

TABLE XXVII

| | % Inhibition | | | Expected | Actual | Potenti- | Degree of |
|---|---|---|---|---|---|---|---|
| Day | G2 | G4 | G6 | Response | Response | ation | Response |
| 19 | −1.0 | −12.8 | 8.0 | −13.8 | 5.8 | no | — |
| 22 | 12.5 | −24.9 | −12.8 | −12.4 | −0.4 | no | — |
| 26 | 11.7 | 19.5 | 22.9 | 31.2 | 8.3 | no | — |
| 29 | −20.5 | −12.8 | 12.8 | −33.3 | 46.1 | yes | Greater |
| | | | | | | | than |
| | | | | | | | additive |
| 34 | −2.7 | 43.9 | 39.1 | 41.2 | −2.1 | no | — |
| 37 | −23.5 | 60.5 | 81.7 | 37.0 | 44.7 | yes | Greater |
| | | | | | | | than |
| | | | | | | | additive |
| 41 | −42.2 | 56.9 | 70.1 | 14.7 | 55.4 | yes | Greater |
| | | | | | | | than |
| | | | | | | | additive |
| 44 | −20.5 | 38.3 | 41.8 | 17.8 | 24.0 | yes | Greater |
| | | | | | | | than |
| | | | | | | | additive |
| 48 | −37.8 | 41.9 | 43.6 | 4.1 | 39.5 | yes | Greater |
| | | | | | | | than |
| | | | | | | | additive |
| 51 | 25.2 | 46.0 | 47.4 | 20.8 | 26.6 | yes | Greater |
| | | | | | | | than |
| | | | | | | | additive |

Table XXVIII shows the % of tumor growth inhibition of aplidine and sunitinib administered as single agents and in combination against A498 human renal tumor xenograft at a dose of 0.06 mg/kg/day of aplidine and 30 mg/kg/day of sunitinib. Additionally, the potentiation and the degree of additivity of the combination of aplidine with sunitinib at said doses are provided.

TABLE XXVIII

| | % Inhibition | | | Expected | Actual | Potenti- | Degree of |
|---|---|---|---|---|---|---|---|
| Day | G2 | G5 | G7 | Response | Response | ation | Response |
| 19 | −1.0 | −17.3 | −2.9 | −18.3 | 15.4 | yes | Greater |
| | | | | | | | than |
| | | | | | | | additive |
| 22 | 12.5 | −15.8 | 14.7 | −3.3 | 18.0 | yes | Greater |
| | | | | | | | than |
| | | | | | | | additive |
| 26 | 11.7 | −11.6 | 20.8 | 0.1 | 20.7 | yes | Greater |
| | | | | | | | than |
| | | | | | | | additive |
| 29 | −20.5 | −36.0 | 7.3 | −56.4 | 63.7 | yes | Greater |
| | | | | | | | than |
| | | | | | | | additive |
| 34 | −2.7 | −4.0 | 24.0 | 6.7 | 30.7 | yes | Greater |
| | | | | | | | than |
| | | | | | | | additive |
| 37 | −23.5 | 11.0 | 45.0 | −12.5 | 57.5 | yes | Greater |
| | | | | | | | than |
| | | | | | | | additive |
| 41 | −42.2 | 3.5 | 35.0 | −38.8 | 73.8 | yes | Greater |
| | | | | | | | than |
| | | | | | | | additive |
| 44 | −20.5 | 8.1 | 23.9 | −12.4 | 36.3 | yes | Greater |
| | | | | | | | than |
| | | | | | | | additive |
| 48 | −37.8 | 15.1 | 23.7 | −22.7 | 46.4 | yes | Greater |
| | | | | | | | than |
| | | | | | | | additive |
| 51 | 25.2 | 18.2 | 31.5 | −7.1 | 38.6 | yes | Greater |
| | | | | | | | than |
| | | | | | | | additive |

Table XXIX shows the % of tumor growth inhibition of aplidine and sunitinib administered as single agents and in combination against A498 human renal tumor xenograft at a dose of 0.04 mg/kg/day of aplidine and 40 mg/kg/day of sunitinib. Additionally, the potentiation and the degree of additivity of the combination of aplidine with sunitinib at said doses are provided.

TABLE XXIX

| | % Inhibition | | | Expected | Actual | Potenti- | Degree of |
|---|---|---|---|---|---|---|---|
| Day | G3 | G4 | G8 | Response | Response | ation | response |
| 19 | −13.0 | −12.8 | 0.8 | −25.8 | 26.6 | yes | Greater |
| | | | | | | | than |
| | | | | | | | additive |
| 22 | −9.5 | −24.9 | 6.3 | −34.4 | 40.7 | yes | Greater |
| | | | | | | | than |
| | | | | | | | additive |
| 26 | −16.0 | 19.5 | 8.8 | 3.5 | 5.3 | no | — |
| 29 | −11.9 | −12.8 | 0.5 | −24.7 | 25.2 | yes | Greater |
| | | | | | | | than |
| | | | | | | | additive |
| 34 | 0.6 | 43.9 | 20.1 | 44.5 | −24.3 | no | — |
| 37 | −33.2 | 60.5 | 49.6 | 27.3 | 22.3 | no | — |
| 41 | −14.2 | 56.9 | 42.2 | 42.7 | −0.5 | no | — |
| 44 | −18.1 | 38.3 | 34.1 | 20.2 | 13.9 | no | — |
| 48 | −11.6 | 41.9 | 32.2 | 30.3 | 1.9 | no | — |
| 51 | −2.5 | 46.0 | 41.8 | 43.5 | −1.7 | no | — |

Aplidine administered as single agent (monotherapy) had no effect on the inhibition of tumor growth at the dose, route and schedule of administration tested on this study. Additionally, sunitinib administered as single agent (monotherapy) at a dose of 40 mg/kg resulted in a trend toward activity on tumor growth inhibition, however this level of activity did not reach the NCI criteria. These treatments did not cause a significant decline in mean body weight, and all mice gained weight by the end of the study.

On the contrary, a greater than additive potentiation of antitumor activity was observed when aplidine, at a dose of 0.060 mg/kg/day, was combined with sunitinib, at a dose of 40 or 30 mg/kg/day.

Finally, the combination of aplidine with sunitinib caused an acceptable decline in body weight, with a maximum of 13% weight loss.

### Example 3

### In Vivo Study to Determine the Effect of Aplidine in Combination with Temsirolimus in a Human Renal Tumor Xenograft

The aim of this study was to evaluate the ability of aplidine to potentiate the antitumoral activity of temsirolimus by using a xenograft model of human renal cancer.

Female athymic nude mice (Harlan Sprague Dawley, Madison, Wis.) were utilized for all experiments. Mice were implanted with tumor fragments when mice were 5 weeks of age. Animals were housed in ventilated rack caging, 5 mice per cage, with food and water ad libitum. The mice were acclimated for 1 week prior to being implanted with tumor fragments. The Vehicle Control group contained 15 mice and the treated groups had each 10 mice/group.

The tumor model used in this study was MRI-H-121 cell line, which is a human kidney carcinoma cell line originally obtained from the DCT Tumor Bank. For MRI-H-121 assay, animals were implanted subcutaneously (SC) on the right flank, using a trocar, with 3 mm³ of tissue tumor fragments, from an in vivo transplantable line passage 1, using sterile Earle's Balanced Salt solution as a wetting agent. Bacterial cultures taken at the implantation time were negative for contamination at both 24 and 48 hours post-implant.

Tumor measurements were determined as disclosed in Example 1. When tumor growth was within an average size range of 95±1.6 mg (mean±SD), mice were randomized into the treatment and control groups based on tumor weight by using LabCat® In Life module V 8.0 SP1 tumor tracking and measurement software.

The treatment of MRI-H-121-tumor-bearing mice were initiated on DPI (Day Post Implantation) 12. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Temsirolimus was provided in the form of vials containing a non-aqueous, ethanolic, sterile solution of temsirolimus, 25 mg/ml. This solution was diluted with a diluent solution containing polysorbate 80 (40% w/v), polyethylene glycol 400 (42.8% w/v) and dehydrated alcohol (19.9% w/v). Then, the solution was further diluted in 0.9% Saline to the dosing formulation concentrations.

Study groups and treatment regimens for the xenograft model are listed in table XXX.

TABLE XXX

| | N of | | | | Sched- | Test |
|---|---|---|---|---|---|---|
| Group | Animals | Dose | | Route | ule | material |
| G1 | 15 | 10 | ml/kg/day | IP | A | CEW (0.18/ |
| (Control | | | | | | 0.18/0.84) |
| group) | | | | | | |
| G2 | 10 | 0.060 | mg/kg/day | IP | A | aplidine |
| G3 | 10 | 20 | mg/kg/day | IP | B | temsirolimus |
| G4 | 10 | 10 | mg/kg/day | IP | B | temsirolimus |
| G5 | 10 | 0.060 | mg/kg/day | IP | A | aplidine |
| | | 20 | mg/kg/day | IP | B | temsirolimus |
| G6 | 10 | 0.060 | mg/kg/day | IP | A | aplidine |
| | | 10 | mg/kg/day | IP | B | temsirolimus |

IP: Intraperitoneal administration
A: Two cycles wherein test material is firstly administered every day for 9 consecutive days, and secondly every day for 5 consecutive days, with a rest period of 5 days between cycles
B: Three cycles wherein test material is administered every day for 5 consecutive days with a rest period of 2 days between cycles

Tumor size measurements were recorded twice weekly from the treatment initiation until the termination of the study. Tumor growth inhibition was assessed comparing the mean tumor weight between the two agents in combination (aplidine and temsirolimus) against temsirolimus mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 1.

Body weight effects were determined by comparing each mouse body weight measurement with the initial body weight (first day of treatment). The NCI activity criterion of body weight loss >20% was used to gauge compound toxicity.

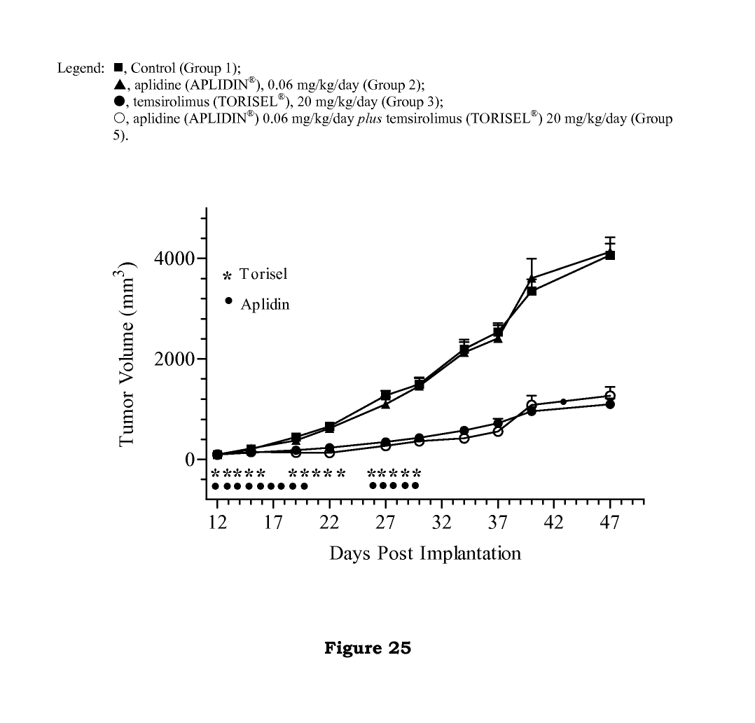

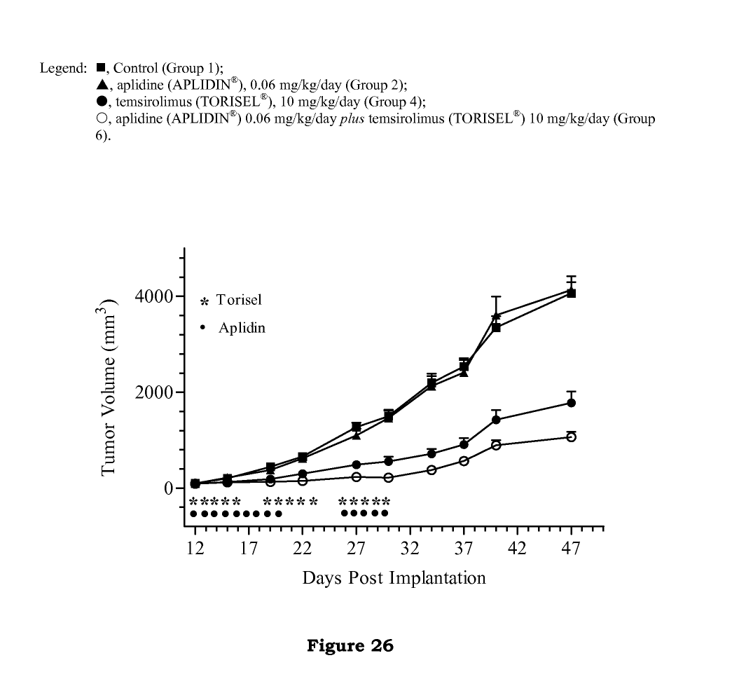

Table XXXI reports the % T/C values obtained with each of the treatments and FIGS. 25-26 show the tumor volume evolution (mean±SEM) of MRI-H-121 tumors in mice treated with control (vehicle), aplidine, temsirolimus, or aplidine plus temsirolimus at different doses.

TABLE XXXI

| | % T/C on day | | | | | | | | | |
|---|---|---|---|---|---|---|---|---|---|---|
| Group | 12 | 15 | 19 | 22 | 27 | 30 | 34 | 37 | 40 | 47 |
| G1 | — | — | — | — | — | — | — | — | — | — |
| (Control | | | | | | | | | | |
| group) | | | | | | | | | | |
| G2 | 98.3 | 109.0 | 84.9 | 94.0 | 86.3 | 97.5 | 97.0 | 95.0 | 107.6 | 101.6 |
| G3 | 97.7 | 66.8 | 41.5 | 35.1 | 27.4 | 28.5 | 26.3 | 28.4 | 28.5 | 27.0 |
| G4 | 95.8 | 62.2 | 42.3 | 45.7 | 38.3 | 36.9 | 32.6 | 35.8 | 42.5 | 43.8 |
| G5 | 98.2 | 74.8 | 30.2 | 19.9 | 21.4 | 24.0 | 19.1 | 21.9 | 32.3 | 31.1 |
| G6 | 95.6 | 59.1 | 29.7 | 22.5 | 18.1 | 14.5 | 17.2 | 22.2 | 26.7 | 26.1 |

Table XXXII shows the % of tumor growth inhibition of aplidine and temsirolimus administered as single agents and in combination against MRI-H-121 human renal tumor xenograft at a dose of 0.06 mg/kg/day of aplidine and 20 mg/kg/day of temsirolimus. Additionally, the potentiation and the degree of additivity of the combination of aplidine with temsirolimus at said doses are provided.

TABLE XXXII

| | % Inhibition | | | Expected | Actual | Potenti- | Degree of |
|---|---|---|---|---|---|---|---|
| Day | G2 | G3 | G5 | Response | Response | ation | Response |
| 12 | 1.7 | 2.3 | 1.8 | 4.0 | −2.2 | No | — |
| 15 | −9.0 | 33.2 | 25.2 | 24.2 | 1.0 | No | — |
| 19 | 15.1 | 58.5 | 69.8 | 73.6 | −3.8 | Yes | Less than |
| | | | | | | | additive |
| 22 | 6.0 | 64.9 | 80.1 | 70.9 | 9.2 | Yes | Additive |
| 27 | 13.7 | 72.6 | 78.6 | 86.3 | −7.7 | Yes | Less than |
| | | | | | | | additive |
| 30 | 2.5 | 71.5 | 76.0 | 74.0 | 2.0 | Yes | Additive |
| 34 | 3.0 | 73.7 | 80.9 | 76.7 | 4.2 | Yes | Additive |
| 37 | 5.0 | 71.6 | 78.1 | 76.6 | 1.5 | Yes | Additive |
| 40 | −7.6 | 71.5 | 67.7 | 63.9 | 3.8 | No | — |
| 47 | −1.6 | 73.0 | 68.9 | 71.4 | −2.5 | No | — |

Table XXXIII shows the % of tumor growth inhibition of aplidine and temsirolimus administered as single agents and in combination against MRI-H-121 human renal tumor xenograft at a dose of 0.06 mg/kg/day of aplidine and 10 mg/kg/day of temsirolimus. Additionally, the potentiation and the degree of additivity of the combination of aplidine with temsirolimus at said doses are provided.

TABLE XXXIII

| | % Inhibition | | | Expected | Actual | Potenti- | Degree of |
|---|---|---|---|---|---|---|---|
| Day | G2 | G3 | G5 | Response | Response | ation | Response |
| 12 | 1.7 | 4.2 | 4.4 | 5.9 | −1.5 | Yes | Less than |
| | | | | | | | additive |
| 15 | −9.0 | 37.8 | 40.9 | 28.8 | 12.1 | Yes | Greater |
| | | | | | | | then |
| | | | | | | | additive |
| 19 | 15.1 | 57.7 | 70.3 | 72.8 | −2.5 | Yes | Additive |
| 22 | 6.0 | 54.3 | 77.5 | 60.3 | 17.2 | Yes | Greater |
| | | | | | | | then |
| | | | | | | | additive |
| 27 | 13.7 | 61.7 | 81.9 | 75.4 | 6.5 | Yes | Additive |
| 30 | 2.5 | 63.1 | 85.5 | 65.6 | 19.9 | Yes | Greater |
| | | | | | | | then |
| | | | | | | | additive |
| 34 | 3.0 | 67.4 | 82.8 | 70.4 | 12.4 | Yes | Greater |
| | | | | | | | then |
| | | | | | | | additive |
| 37 | 5.0 | 64.2 | 77.8 | 69.2 | 8.6 | Yes | Additive |
| 40 | −7.6 | 57.5 | 73.3 | 49.9 | 23.4 | Yes | Greater |
| | | | | | | | then |
| | | | | | | | additive |
| 47 | −1.6 | 56.2 | 73.9 | 54.6 | 19.3 | Yes | Greater |
| | | | | | | | then |
| | | | | | | | additive |

MRI-H-121 xenograft tumors were refractory to aplidine therapy at the doses, schedule and route of administration tested in this experiment. Xenograft tumors were very sensitive to therapy with temsirolimus administered at both 10 and 20 mg/kg/day, with the higher dose providing too much activity to properly evaluate the effect of the combination of aplidine at 0.06 mg/kg/day plus temsirolimus at 20 mg/kg/day.

However, when aplidine at 0.06 mg/kg/day was combined with temsirolimus at 10 mg/kg/day, a statistically significant potentiation of activity was observed. This potentiation was determined to be greater than additive. The therapeutic combination of aplidine plus temsirolimus was well tolerated by the mice without any evidence of additional toxicity.

### Example 4

### In Vivo Study of Aplidine in Combination with Temsirolimus in a Human Renal Tumor Xenograft CAKI-1

Female athymic nude mice (Harlan Sprague Dawley, Madison, Wis.) were utilized for all experiments. Mice were implanted with a cell suspension when mice were 6-8 weeks of age. Animals were housed in ventilated rack caging with food and water ad libitum. The mice were acclimated for at least 5 days prior to tumor implantation. The Vehicle Control group contained 15 mice and the treated groups each had 10 mice/group. The tumor model used in this study was CAKI-1, which was implanted in the animals as disclosed in Example 2.

Tumor measurements were determined as disclosed in Example 1. When tumor growth was within an average size range of 140±34 mg (mean±SD), mice were randomized into the treatment and control groups based on tumor weight by using LabCat® In Life module V 8.0 SP1 tumor tracking and measurement software.

The treatment of CAKI-1-tumor-bearing mice was initiated on DPI (Day Post Implantation) 21. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Temsirolimus was provided in the form of vials containing a non-aqueous, ethanolic, sterile solution of temsirolimus, 25 mg/ml. This solution was diluted with a diluent solution containing polysorbate 80 (40% w/v), polyethylene glycol 400 (42.8% w/v) and dehydrated alcohol (19.9% w/v). Then, the solution was further diluted in 0.9% Saline to the dosing formulation concentrations.

Study groups and treatment regimens for the three xenograft models are listed in table XXXIV.

TABLE XXXIV

| | N of | | | | Sched- | Test |
|---|---|---|---|---|---|---|
| Group | Animals | Dose | | Route | ule | material |
| G1 | 15 | 10 | ml/kg/day | IP | Qdx9x2 | CEW (0.18/ |
| (Control | | | | | | 0.18/0.84) |
| group) | | 10 | ml/kg/day | IP | Qdx5x2 | 0.9% Saline |
| G2 | 10 | 0.060 | mg/kg/day | IP | Qdx9x2 | aplidine |
| G3 | 10 | 20 | mg/kg/day | IP | Qdx5x2 | temsirolimus |
| G4 | 10 | 10 | mg/kg/day | IP | Qdx5x2 | temsirolimus |
| G5 | 10 | 0.060 | mg/kg/day | IP | Qdx9x2 | aplidine |
| | | 20 | mg/kg/day | IP | Qdx5x2 | temsirolimus |
| G6 | 10 | 0.060 | mg/kg/day | IP | Qdx9x2 | aplidine |
| | | 10 | mg/kg/day | IP | Qdx5x2 | temsirolimus |

IP: Intraperitoneal administration
Qdx9x2: Two cycles wherein test material is administered every day for 9 consecutive days with a rest period of 6 days between cycles
Qdx5x2: Two cycles wherein test material is administered every day for 5 consecutive days with a rest period of 4 days between cycles

Tumor size measurements were recorded twice weekly from the treatment initiation until the termination of the study. Tumor growth inhibition was assessed comparing the mean tumor weight between the two agents in combination (aplidine and temsirolimus) against temsirolimus mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclosed in Example 1.

Body weight effects were determined by comparing each mouse body weight measurement with the initial body weight (first day of treatment). The NCI activity criterion of body weight loss >20% was used to gauge compound toxicity.

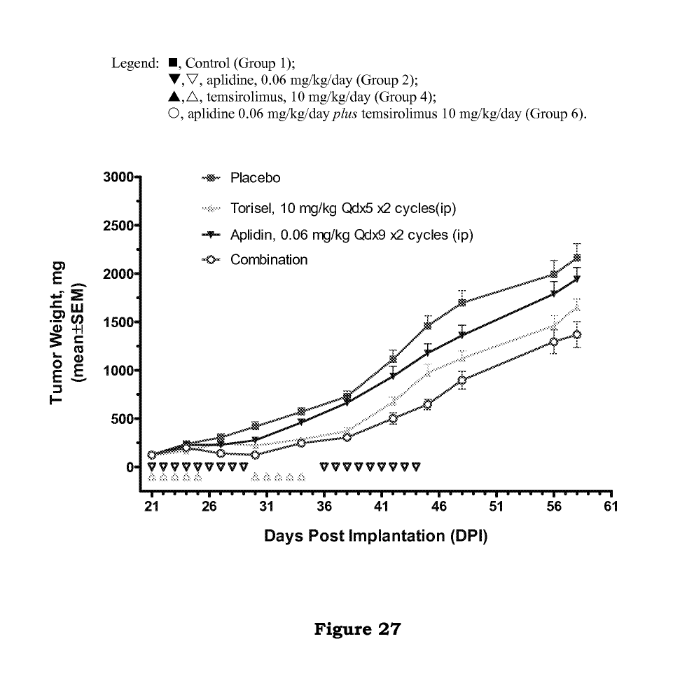

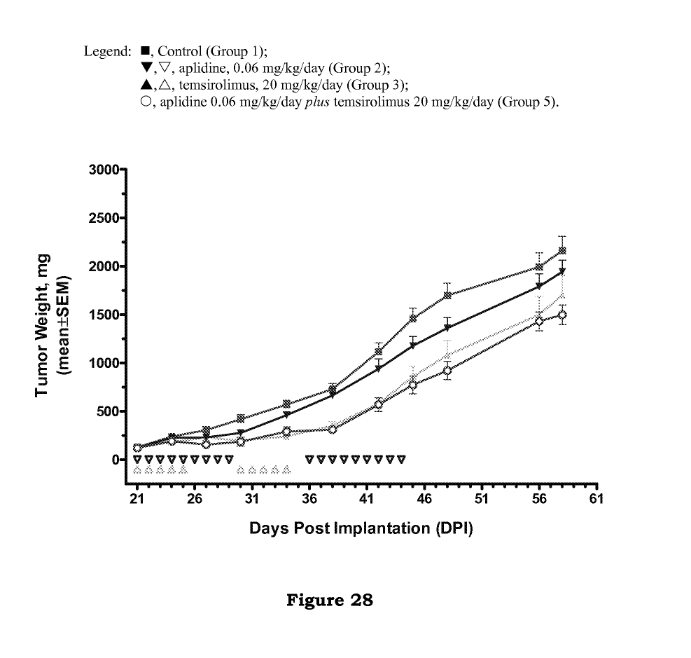

Table XXXV reports the % T/C values obtained with each of the treatments and FIGS. 27 and 28 show the tumor weight evolution (mean±SEM) of CAKI-1 tumors in mice treated with control (vehicle), aplidine, temsirolimus, or aplidine plus temsirolimus at different doses.

TABLE XXXV

% T/C on day

| Group | 21 | 24 | 27 | 30 | 34 | 38 |
|---|---|---|---|---|---|---|
| G1 | — | — | — | — | — | — |
| (Control | | | | | | |
| group) | | | | | | |
| G2 | 98.2 | 94.6 | 75.6 | 66.0 | 81.0 | 91.1 |
| G3 | 98.3 | 76.7 | 75.3 | 47.0 | 42.2 | 48.0 |
| G4 | 95.7 | 69.9 | 81.3 | 53.2 | 50.4 | 51.1 |
| G5 | 96.0 | 79.8 | 50.5 | 44.2 | 51.1 | 42.5 |
| G6 | 97.7 | 82.9 | 46.1 | 29.7 | 43.3 | 41.9 |

% T/C on day

| Group | 42 | 45 | 48 | 56 | 58 |
|---|---|---|---|---|---|
| G1 | — | — | — | — | — |
| (Control | | | | | |
| group) | | | | | |
| G2 | 84.1 | 80.7 | 80.1 | 89.8 | 89.9 |
| G3 | 51.9 | 58.9 | 63.6 | 75.3 | 78.8 |
| G4 | 60.3 | 66.7 | 66.2 | 73.4 | 76.5 |
| G5 | 51.0 | 53.0 | 54.2 | 71.8 | 69.3 |
| G6 | 44.9 | 44.3 | 52.8 | 65.0 | 63.3 |

Table XXXVI shows the % of tumor growth inhibition of aplidine and temsirolimus administered as single agents and in combination against CAKI-1 human renal tumor xenograft at a dose of 0.06 mg/kg/day of aplidine and 20 mg/kg/day of temsirolimus. Additionally, the potentiation and the degree of additivity of the combination of aplidine with temsirolimus at said doses are provided.

TABLE XXXVI

| | % Inhibition | | | Expected | Actual | Potenti- | Degree of |
|---|---|---|---|---|---|---|---|
| Day | G2 | G3 | G5 | Response | Response | ation | Response |
| 21 | 1.8 | 1.7 | 4.0 | 3.5 | 0.5 | No | — |
| 24 | 5.4 | 23.3 | 20.2 | 28.7 | −8.5 | No | — |
| 27 | 24.4 | 24.7 | 49.5 | 49.1 | 0.4 | Yes | Additive |
| 30 | 34.0 | 53.0 | 55.8 | 87.0 | −31.2 | Yes | Less than |
| | | | | | | | additive |
| 34 | 19.0 | 57.8 | 48.9 | 76.8 | −27.9 | No | — |
| 38 | 8.9 | 52.0 | 57.5 | 60.9 | −3.4 | Yes | Additive |
| 42 | 15.9 | 48.1 | 49.0 | 64.0 | −15.0 | Yes | Less than |
| | | | | | | | additive |
| 45 | 19.3 | 41.1 | 47.0 | 60.4 | −13.4 | Yes | Less than |
| | | | | | | | additive |
| 48 | 19.9 | 36.4 | 45.8 | 56.3 | −10.5 | Yes | Less than |
| | | | | | | | additive |
| 56 | 10.2 | 24.7 | 28.2 | 34.9 | −6.7 | Yes | Less than |
| | | | | | | | additive |
| 58 | 10.1 | 21.2 | 30.7 | 31.3 | −0.6 | Yes | Additive |

Table XXXVII shows the % of tumor growth inhibition of aplidine and temsirolimus administered as single agents and in combination against CAKI-1 human renal tumor xenograft at a dose of 0.06 mg/kg/day of aplidine and 10 mg/kg/day of temsirolimus. Additionally, the potentiation and the degree of additivity of the combination of aplidine with temsirolimus at said doses are provided.

TABLE XXXVII

| | % Inhibition | | | Expected | Actual | Potenti- | Degree of |
|---|---|---|---|---|---|---|---|
| Day | G2 | G4 | G6 | Response | Response | ation | Response |
| 21 | 1.8 | 4.3 | 2.3 | 6.1 | −3.8 | No | — |
| 24 | 5.4 | 30.1 | 17.1 | 35.5 | −18.4 | No | — |
| 27 | 24.4 | 18.7 | 53.9 | 43.1 | 10.8 | Yes | Greater |
| | | | | | | | than |
| | | | | | | | additive |
| 30 | 34.0 | 46.8 | 70.3 | 80.8 | −10.5 | Yes | Less than |
| | | | | | | | additive |
| 34 | 19.0 | 49.6 | 56.7 | 68.6 | −11.9 | Yes | Less than |
| | | | | | | | additive |
| 38 | 8.9 | 48.9 | 58.1 | 57.8 | 0.3 | Yes | Additive |
| 42 | 15.9 | 39.7 | 55.1 | 55.6 | −0.5 | Yes | Additive |
| 45 | 19.3 | 33.3 | 55.7 | 52.6 | 3.1 | Yes | Additive |
| 48 | 19.9 | 33.8 | 47.2 | 53.7 | −6.5 | Yes | Less than |
| | | | | | | | additive |
| 56 | 10.2 | 26.6 | 35.0 | 36.8 | −1.8 | Yes | Less than |
| | | | | | | | additive |
| 58 | 10.1 | 23.5 | 36.7 | 33.6 | 3.1 | Yes | Additive |

Aplidine was inactive when it was administered as single agent (monotherapy) against CAKI-1 human renal tumor cell line. In addition, treatment with temsirolimus as single agent at doses of 10 and 20 mg/kg/day showed activity against CAKI-1 cell line based on dose response but it did not meet the NCI criteria for activity. Additionally, these treatments did not cause a significant decline in mean body weight, and all mice gained weight by the end of the study.

However, when aplidine, at a dose of 0.060 mg/kg/day, was combined with temsirolimus at a dose of 10 mg/kg/day, a potentiation of activity was observed, resulting in an additive potentiation of tumor growth inhibition. In addition, the combination of aplidine with temsirolimus caused an acceptable decline in body weight, and all mice gained weight by the end of the study.

### Example 5

### In Vivo Study of Aplidine in Combination with Temsirolimus in a Human Non-Small Cell Lung Cancer (NSCLC) Xenograft

Female athymic nude mice (Harlan Sprague Dawley, Madison, Wis.) were utilized for all experiments. Mice were implanted with a cell suspension when mice were 6-8 weeks of age. Animals were housed in ventilated rack caging with food and water ad libitum. The mice were acclimated for at least 5 days prior to tumor implantation. The Vehicle Control group contained 15 mice and the treated groups each had 10 mice/group.

The tumor model used in this study was NCI-H-460, which is a human NSCLC cell line obtained from the ATCC (Manassas, Va.). NCI-H-460 cells were grown in RPMI-1640 medium, 10% FBS, 10 mM Hepes, 1 mM sodium pyruvate, 4.5 g/l glucose, 1.5 g/l sodium bicarbonate and 2 mM L-glutamine. Cells from in vitro passage 11 were implanted SC into study mice: 5×10⁶ cells/mouse in 0.2 ml 50% Matrigel/50% medium without antibiotics or serum, using a 23 G needle and 1 cc syringe.

Bacterial cultures were performed on aliquots of the cells prepared for implantation. All cultures were negative for bacterial contamination at both 24 and 48 hours post-implant.

Tumor measurements were determined as disclosed in Example 1. When tumor growth was within an average size range of 110±25 mg (mean±SD), mice were randomized into the treatment and control groups based on tumor weight by using LabCat® In Life module V 8.0 SP1 tumor tracking and measurement software.

The treatment of NCI-H-460-tumor-bearing mice was initiated on DPI (Day Post Implantation) 7. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Temsirolimus was provided in the form of vials containing a non-aqueous, ethanolic, sterile solution of temsirolimus, 25 mg/ml. This solution was diluted with a diluent solution containing polysorbate 80 (40% w/v), polyethylene glycol 400 (42.8% w/v) and dehydrated alcohol (19.9% w/v). Then, the solution was further diluted in 0.9% Saline to the dosing formulation concentrations.

Study groups and treatment regimens for the three xenograft models are listed in table XXXVIII.

TABLE XXXVIII

| | N of | | | | Sched- | Test |
|---|---|---|---|---|---|---|
| Group | Animals | Dose | | Route | ule | material |
| G1 | 15 | 10 | ml/kg/day | IP | Qdx9x2 | CEW (0.18/ |
| (Control | | | | | | 0.18/0.84) |
| group) | | 10 | ml/kg/day | IP | Qdx5x2 | 0.9% Saline |
| G2 | 10 | 0.060 | mg/kg/day | IP | Qdx9x2 | aplidine |
| G3 | 10 | 20 | mg/kg/day | IP | Qdx5x2 | temsirolimus |
| G4 | 10 | 10 | mg/kg/day | IP | Qdx5x2 | temsirolimus |
| G5 | 10 | 0.060 | mg/kg/day | IP | Qdx9x2 | aplidine |
| | | 20 | mg/kg/day | IP | Qdx5x2 | temsirolimus |
| G6 | 10 | 0.060 | mg/kg/day | IP | Qdx9x2 | aplidine |
| | | 10 | mg/kg/day | IP | Qdx5x2 | temsirolimus |

IP: Intraperitoneal administration
Qdx9x2: Two cycles wherein test material is administered every day for 9 consecutive days with a rest period of 5 days between cycles
Qdx5x2: Two cycles wherein test material is administered every day for 5 consecutive days with a rest period of 4 days between cycles

Tumor size measurements were recorded twice weekly from the treatment initiation until the termination of the study. Tumor growth inhibition was assessed comparing the mean tumor weight between the two agents in combination (aplidine and temsirolimus) against temsirolimus mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 1.

Body weight effects were determined by comparing each mouse body weight measurement with the initial body weight (first day of treatment). The NCI activity criterion of body weight loss >20% was used to gauge compound toxicity.

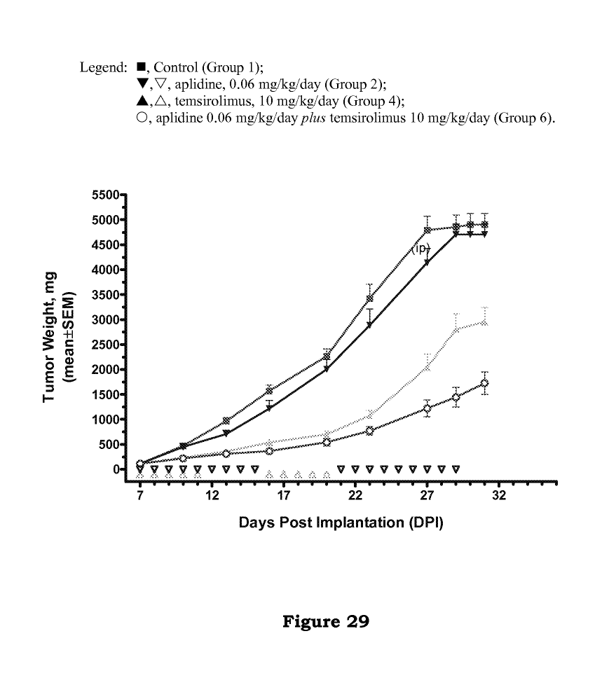

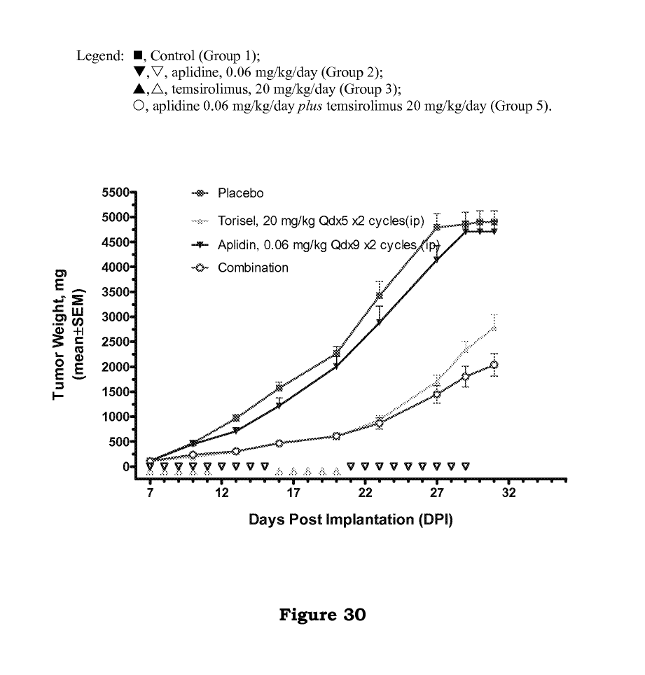

Table XXXIX reports the % T/C values obtained with each of the treatments and FIGS. 29 and 30 show the tumor weight evolution (mean±SEM) of NCI-H-460 tumors in mice treated with control (vehicle), aplidine, temsirolimus, or aplidine plus temsirolimus at different doses.

TABLE XXXIX

| | % T/C on day | | | | | | | | |
|---|---|---|---|---|---|---|---|---|---|
| Group | 7 | 10 | 13 | 16 | 20 | 23 | 27 | 29 | 31 |
| G1 | — | — | — | — | — | — | — | — | — |
| (Control | | | | | | | | | |
| group) | | | | | | | | | |
| G2 | 101.2 | 95.5 | 73.2 | 77.4 | 88.5 | 84.3 | 86.4 | 96.9 | 96.0 |
| G3 | 98.2 | 41.2 | 30.5 | 30.9 | 26.3 | 27.4 | 35.6 | 48.2 | 56.8 |
| G4 | 102.1 | 49.9 | 37.1 | 34.1 | 30.9 | 31.4 | 42.6 | 57.8 | 60.3 |
| G5 | 99.2 | 50.3 | 31.5 | 29.5 | 26.9 | 25.2 | 30.2 | 37.1 | 41.5 |
| G6 | 101.8 | 46.4 | 32.2 | 23.3 | 23.9 | 22.5 | 25.5 | 29.8 | 35.1 |

Table XXXX shows the % of tumor growth inhibition of aplidine and temsirolimus administered as single agents and in combination against NCI-H-460 human NSCLC xenograft at a dose of 0.06 mg/kg/day of aplidine and 20 mg/kg/day of temsirolimus. Additionally, the potentiation and the degree of additivity of the combination of aplidine with temsirolimus at said doses are provided.

TABLE XXXX

| | % Inhibition | | | Expected | Actual | Potenti- | Degree of |
|---|---|---|---|---|---|---|---|
| Day | G2 | G3 | G5 | Response | Response | ation | Response |
| 7 | −1.2 | 1.8 | 0.8 | 0.6 | 0.2 | No | — |
| 10 | 4.5 | 58.8 | 49.7 | 63.3 | −13.6 | No | — |
| 13 | 26.8 | 69.5 | 68.5 | 96.3 | −27.8 | No | — |
| 16 | 22.6 | 69.1 | 70.5 | 91.7 | −21.2 | No | — |
| 20 | 11.5 | 73.7 | 73.1 | 85.2 | −12.1 | No | — |
| 23 | 15.7 | 72.6 | 74.8 | 88.3 | −13.5 | Yes | Less than |
| | | | | | | | additive |
| 27 | 13.6 | 64.4 | 69.8 | 78.0 | −8.2 | Yes | Less than |
| | | | | | | | additive |
| 29 | 3.1 | 51.8 | 62.9 | 54.9 | 8.0 | Yes | Greater |
| | | | | | | | than |
| | | | | | | | additive |
| 31 | 4.0 | 43.2 | 58.5 | 47.2 | 11.3 | Yes | Geater |
| | | | | | | | than |
| | | | | | | | additive |

Table XXXXI shows the % of tumor growth inhibition of aplidine and temsirolimus administered as single agents and in combination against NCI-H-460 human NSCLC xenograft at a dose of 0.06 mg/kg/day of aplidine and 10 mg/kg/day of temsirolimus. Additionally, the potentiation and the degree of additivity of the combination of aplidine with temsirolimus at said doses are provided.

TABLE XXXXI

| | % Inhibition | | | Expected | Actual | Potenti- | Degree of |
|---|---|---|---|---|---|---|---|
| Day | G2 | G4 | G6 | Response | Response | ation | Response |
| 7 | −1.2 | −2.1 | −1.8 | −3.3 | 1.5 | No | — |
| 10 | 4.5 | 50.1 | 53.6 | 54.6 | −1.0 | Yes | Less than |
| | | | | | | | additive |
| 13 | 26.8 | 62.9 | 67.8 | 89.7 | −21.9 | Yes | Less than |
| | | | | | | | additive |
| 16 | 22.6 | 65.9 | 76.7 | 88.5 | −11.8 | Yes | Less than |
| | | | | | | | additive |
| 20 | 11.5 | 69.1 | 76.1 | 80.6 | −4.5 | Yes | Less than |
| | | | | | | | additive |
| 23 | 15.7 | 68.6 | 77.5 | 84.3 | −6.8 | Yes | Less than |
| | | | | | | | additive |
| 27 | 13.6 | 57.4 | 74.5 | 71.0 | 3.5 | Yes | Additive |
| 29 | 3.1 | 42.2 | 70.2 | 45.3 | 24.9 | Yes | Greater |
| | | | | | | | than |
| | | | | | | | additive |
| 31 | 4.0 | 39.7 | 64.9 | 43.7 | 21.2 | Yes | Geater |
| | | | | | | | than |
| | | | | | | | additive |

Aplidine was inactive when was administered as single agent (monotherapy) against NCI-H-460 human NSCLC cell line. In addition, treatment with temsirolimus as single agent at doses of 10 and 20 mg/kg/day showed activity against NCI-H-460 cell line meeting the NCI criteria for activity. Additionally, these treatments did not cause a significant decline in mean body weight, and all mice gained weight by the end of the study.

However, when aplidine, at a dose of 0.060 mg/kg/day, was combined with temsirolimus at both doses of 10 and 20 mg/kg/day, a potentiation of activity was observed, resulting in a greater than additive potentiation of tumor growth inhibition at the end of the study. In addition, the combination of aplidine with temsirolimus caused an acceptable decline in body weight, and all mice gained weight by the end of the study.

### Example 6

### In Vivo Study of Aplidine in Combination with Sorafenib in a Human Hepatoma Xenograft

Female athymic nude mice (Harlan Sprague Dawley, Madison, Wis.) were utilized for all experiments. Mice were implanted with a cell suspension when mice were 6-8 weeks of age. Animals were housed in ventilated rack caging with food and water ad libitum. The mice were acclimated for at least 5 days prior to tumor implantation. The Vehicle Control group contained 15 mice and the treated groups had each 9 mice/group.

The tumor model used in this study was HepG2, which is a human hepatoma cell line obtained from the ATCC (Manassas, Va.). HepG2 cells were grown in MEM medium, 10% FBS, 1.5 g/l sodium bicarbonate, 0.1 mM non-essential amino acids, 1.0 mM sodium pyruvate and 2 mM L-glutamine. Cells from in vitro passage 4-20 were implanted SC into study mice: 5×10⁶ cells/mouse in 0.2 ml 50% Matrigel/50% medium without antibiotics or serum, using a 23 G needle and 1 cc syringe. Bacterial cultures were performed on aliquots of the cells prepared for implantation. All cultures were negative for bacterial contamination at both 24 and 48 hours post-implant.

Tumor measurements were determined as disclosed in Example 1. When tumor growth was within an average size range of 130±44 mg (mean±SD), mice were randomized into the treatment and control groups based on tumor weight by using LabCat® In Life module V 8.0 SP1 tumor tracking and measurement software.

The treatment of HepG2-tumor-bearing mice was initiated on DPI (Day Post Implantation) 19. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Sorafenib was provided in the form of a 200 mg reddish colored tablet containing sorafenib in the form of its tosylate salt. The sorafenib solution was made by solving the tablet in Cremophor EL/ethanol (50/50) to a concentration of 50 mg/ml. Then the solution was diluted in water for infusion (wfi) to a final proportion of CEW of (12.5, 12.5, 75). The sorafenib solution in CEW was diluted in wfi to the dosing formulation concentrations.

Study groups and treatment regimens for the three xenograft models are listed in table XXXXII.

TABLE XXXXII

| | N of | | | | Sched- | Test |
|---|---|---|---|---|---|---|
| Group | Animals | Dose | | Route | ule | material |
| G1 | 15 | 10 | ml/kg/day | IV | A | 0.18% Placebo |
| (Control | | | | | | in Saline |
| group) | | 10 | ml/kg/day | PO | B | CEW (12.5/ |
| | | | | | | 12.5/75) |
| G2 | 9 | 0.060 | mg/kg/day | IP | C | aplidine |
| G3 | 9 | 60 | mg/kg/day | PO | B | sorafenib |
| G4 | 9 | 30 | mg/kg/day | PO | B | sorafenib |
| G5 | 9 | 0.060 | mg/kg/day | IP | C | aplidine |
| | | 60 | mg/kg/day | PO | B | sorafenib |
| G6 | 9 | 0.060 | mg/kg/day | IP | C | aplidine |
| | | 30 | mg/kg/day | PO | B | sorafenib |

IP: Intraperitoneal administration; PO: Oral administration; IV: Intravenous administration
A: DPI 19, 26 and 33; B: DPI 19-34; C: DPI 19-27
Placebo: 500 mg sucrose + 34 mg potassium phosphate + phosphoric acid q.s. pH 3.8-4.4

Tumor size measurements were recorded twice weekly from the treatment initiation until the termination of the study. Tumor growth inhibition was assessed comparing the mean tumor weight between the two agents in combination (aplidine and sorafenib) against sorafenib mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 1.

Body weight effects were determined by comparing each mouse body weight measurement with the initial body weight (first day of treatment). The NCI activity criterion of body weight loss >20% was used to gauge compound toxicity.

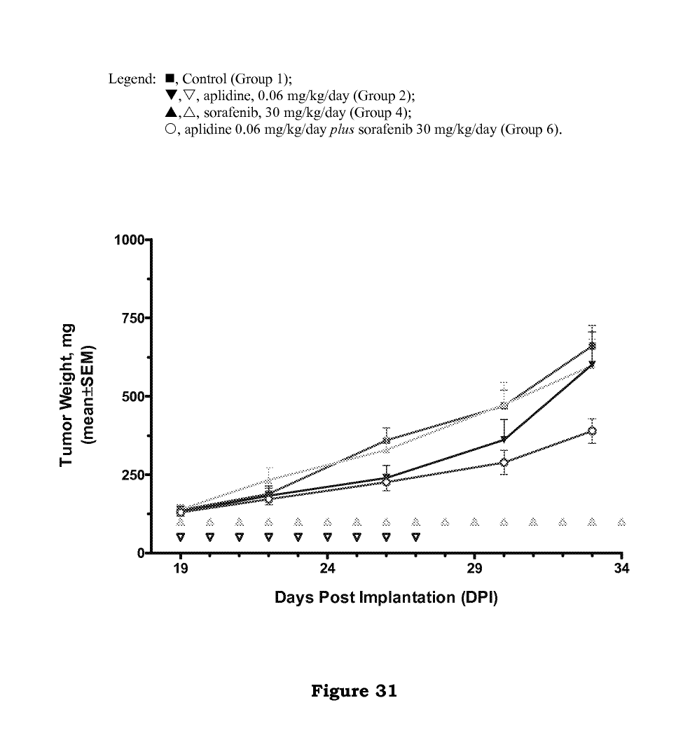

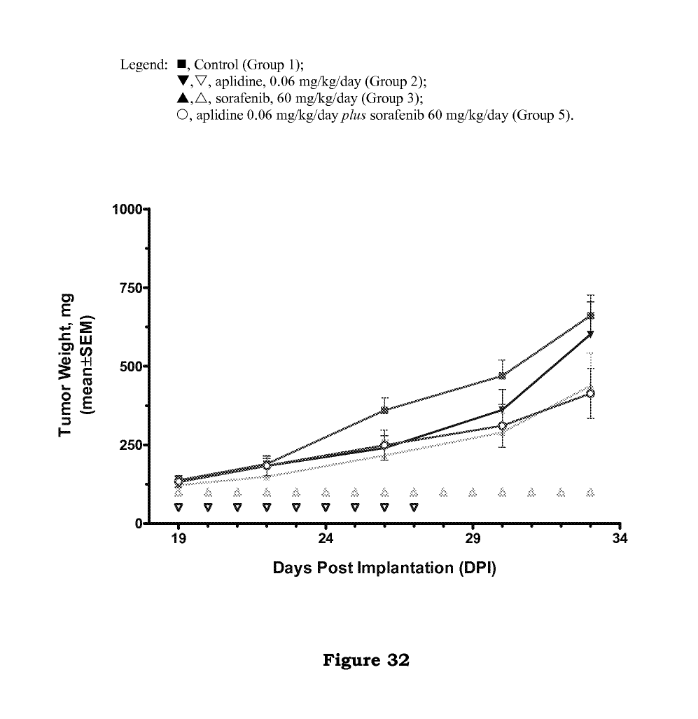

Table XXXXIII reports the % T/C values obtained with each of the treatments and FIGS. 31 and 32 show the tumor weight evolution (mean±SEM) of HepG2 tumors in mice treated with control (vehicle), aplidine, sorafenib, or aplidine plus sorafenib at different doses.

TABLE XXXXIII

| | % T/C on day | | | | |
|---|---|---|---|---|---|
| Group | 19 | 22 | 26 | 30 | 33 |
| G1 | — | — | — | — | — |
| (Control | | | | | |
| group) | | | | | |
| G2 | 95.9 | 97.0 | 66.7 | 76.9 | 91.0 |
| G3 | 88.8 | 78.7 | 60.1 | 61.8 | 66.2 |
| G4 | 101.4 | 123.5 | 91.3 | 100.9 | 90.4 |
| G5 | 97.1 | 97.2 | 69.1 | 66.1 | 62.5 |
| G6 | 94.6 | 90.9 | 62.8 | 61.6 | 58.9 |

Table XXXXIV shows the % of tumor growth inhibition of aplidine and sorafenib administered as single agents and in combination against HepG2 human hepatoma xenograft at a dose of 0.06 mg/kg/day of aplidine and 60 mg/kg/day of sorafenib. Additionally, the potentiation and the degree of additivity of the combination of aplidine with sorafenib at said doses are provided.

TABLE XXXXIV

| | % Inhibition | | | Expected | Actual | Potenti- | Degree of |
|---|---|---|---|---|---|---|---|
| Day | G2 | G3 | G5 | Response | Response | ation | Response |
| 19 | 4.1 | 11.2 | 2.9 | 15.3 | −12.4 | No | — |
| 22 | 3.0 | 21.3 | 2.8 | 24.3 | −21.5 | No | — |
| 26 | 33.3 | 39.9 | 30.9 | 73.2 | −42.3 | No | — |
| 30 | 23.1 | 38.2 | 33.9 | 61.3 | −27.4 | No | — |
| 33 | 9.0 | 33.8 | 37.5 | 42.8 | −5.3 | Yes | Less than |
| | | | | | | | additive |

Table XXXXV shows the % of tumor growth inhibition of aplidine and sorafenib administered as single agents and in combination against HepG2 human hepatoma xenograft at a dose of 0.06 mg/kg/day of aplidine and 30 mg/kg/day of sorafenib. Additionally, the potentiation and the degree of additivity of the combination of aplidine with sorafenib at said doses are provided.

TABLE XXXXV

| | % Inhibition | | | Expected | Actual | Potenti- | Degree of |
|---|---|---|---|---|---|---|---|
| Day | G2 | G4 | G6 | Response | Response | ation | Response |
| 19 | 4.1 | −1.4 | 5.4 | 2.7 | 2.7 | Yes | Additive |
| 22 | 3.0 | −23.5 | 9.1 | −20.5 | 29.6 | Yes | Greater |
| | | | | | | | than |
| | | | | | | | additive |
| 26 | 33.3 | 8.7 | 37.2 | 42.0 | −4.8 | Yes | Additive |
| 30 | 23.1 | −0.9 | 38.4 | 22.2 | 16.2 | Yes | Greater |
| | | | | | | | than |
| | | | | | | | additive |
| 33 | 9.0 | 9.6 | 41.1 | 18.6 | 22.5 | Yes | Greater |
| | | | | | | | than |
| | | | | | | | additive |

When aplidine and sorafenib were administered as single agents (monotherapy) against HepG2 human hepatoma cell line, they were inactive. Additionally, these treatments did not cause a significant decline in mean body weight.

However, when aplidine, at a dose of 0.060 mg/kg/day, was combined with sorafenib at a dose of 30 mg/kg/day, a potentiation of activity was observed, resulting in a greater than additive potentiation of tumor growth inhibition. In addition, the combination of aplidine with sorafenib caused an acceptable decline in body weight.

### Example 7

### In Vivo Study of Aplidine in Combination with Sorafenib in a Human Melanoma Xenograft

Female athymic nude mice (Harlan Sprague Dawley, Madison, Wis.) were utilized for all experiments. Mice were implanted with a tumor fragment when mice were 6-8 weeks of age. Animals were housed in ventilated rack caging with food and water ad libitum. The mice were acclimated for at least 5 days prior to tumor implantation. The Vehicle Control group contained 15 mice and the treated groups had each 10 mice/group.

The tumor model used in this study was LOX-IMVI, which is a human melanoma cell line obtained from the Department of Developmental Therapeutics, National Cancer Institute (NCI). Animals were implanted subcutaneously (SC) on the right flank, using a 13 gauge trocar, with 2 mm³ of tissue tumor fragments of LOX-IMVI, from an in vivo transplantable line passage 1, using sterile Earle's Balanced Salt solution as a wetting agent. Bacterial cultures taken at the implantation time were negative for contamination at both 24 and 48 hours post-implant.

Tumor measurements were determined as disclosed in Example 1. When tumor growth was within an average size range of 130±44 mg (mean±SD), mice were randomized into the treatment and control groups based on tumor weight by using LabCat® In Life module V 8.0 SP1 tumor tracking and measurement software.

The treatment of LOX-IMVI-tumor-bearing mice was initiated on DPI (Day Post Implantation) 11. Body weights were recorded on treatment days and when tumor sizes were measured. In those days wherein the two drugs were administered the same day, the combination therapy groups were treated by co-administering the two drugs at the same time, with no attempt to sequence the treatments.

Aplidine was provided in the form of vials of lyophilized aplidine powder which was reconstituted in Cremophor EL/ethanol/water (CEW) 15/15/70 to a concentration of 0.5 mg/ml. The aplidine solution in CEW was diluted in 0.9% Saline to the dosing formulation concentrations, being the final proportion of CEW of 0.18/0.18/0.84.

Sorafenib was provided in the form of a 200 mg reddish colored tablet containing sorafenib in the form of its tosylate salt. The sorafenib solution was made by solving the tablet in Cremophor EL/ethanol (50/50) to a concentration of 50 mg/ml. Then the solution was diluted in water for infusion (wfi) to a final proportion of CEW of (12.5, 12.5, 75). The sorafenib solution in CEW was diluted in wfi to the dosing formulation concentrations.

Study groups and treatment regimens for the three xenograft models are listed in table XXXXVI.

TABLE XXXXVI

| | N of | | | | Sched- | Test |
|---|---|---|---|---|---|---|
| Group | Animals | Dose | | Route | ule | material |
| G1 | 15 | 10 | ml/kg/day | IP | A | CEW (0.18/ |
| (Control | | | | | | 0.18/0.84) |
| group) | | 10 | ml/kg/day | PO | B | CEW (12.5/ |
| | | | | | | 12.5/75) |
| G2 | 10 | 0.060 | mg/kg/day | IP | A | aplidine |
| G3 | 10 | 60 | mg/kg/day | PO | B | sorafenib |
| G4 | 10 | 30 | mg/kg/day | PO | B | sorafenib |
| G5 | 10 | 0.060 | mg/kg/day | IP | A | aplidine |
| | | 60 | mg/kg/day | PO | B | sorafenib |
| G6 | 10 | 0.060 | mg/kg/day | IP | A | aplidine |
| | | 30 | mg/kg/day | PO | B | sorafenib |

IP: Intraperitoneal administration; PO: Oral administration
A: DPI 11-19; B: DPI 11-25

Tumor size measurements were recorded twice weekly from the treatment initiation until the termination of the study. Tumor growth inhibition was assessed comparing the mean tumor weight between the two agents in combination (aplidine and sorafenib) against sorafenib mean tumor weight at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 1.

Body weight effects were determined by comparing each mouse body weight measurement with the initial body weight (first day of treatment). The NCI activity criterion of body weight loss >20% was used to gauge compound toxicity.

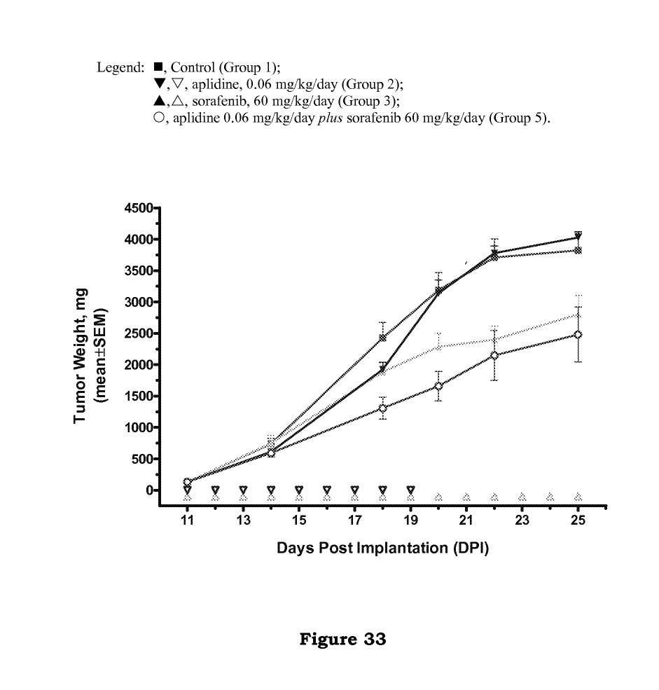

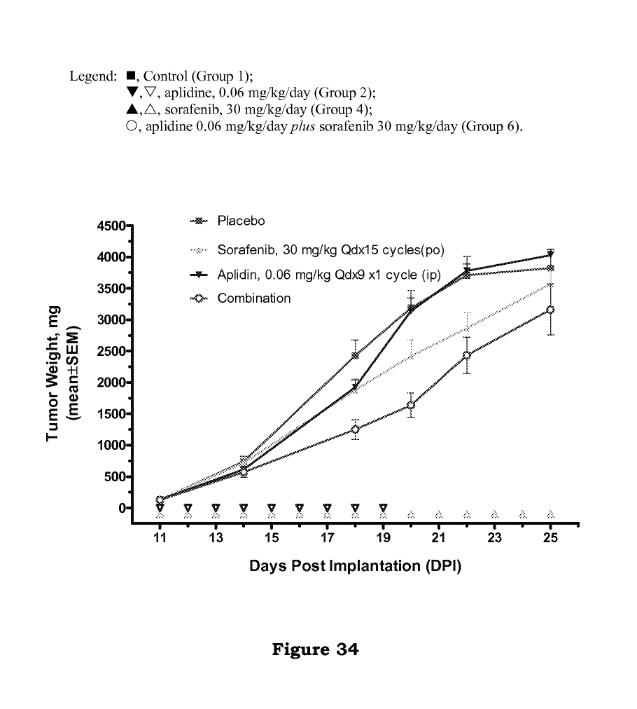

Table XXXXVII reports the % T/C values obtained with each of the treatments and FIGS. 33 and 34 show the tumor weight evolution (mean±SEM) of LOX-IMVI tumors in mice treated with control (vehicle), aplidine, sorafenib, or aplidine plus sorafenib at different doses.

TABLE XXXXVII

% T/C on day

| Group | 11 | 14 | 18 | 20 | 22 | 25 |
|---|---|---|---|---|---|---|
| G1 | — | — | — | — | — | — |
| (Control | | | | | | |
| group) | | | | | | |
| G2 | 102.9 | 82.9 | 79.3 | 98.5 | 102.0 | 105.4 |
| G3 | 98.6 | 101.2 | 77.7 | 71.7 | 64.8 | 73.3 |
| G4 | 102.1 | 96.9 | 77.4 | 75.9 | 77.3 | 93.4 |
| G5 | 100.9 | 80.0 | 53.9 | 52.0 | 57.9 | 64.9 |
| G6 | 97.3 | 76.7 | 51.5 | 51.3 | 65.6 | 82.7 |

Table XXXXVIII shows the % of tumor growth inhibition of aplidine and sorafenib administered as single agents and in combination against LOX-IMVI human melanoma xenograft at a dose of 0.06 mg/kg/day of aplidine and 60 mg/kg/day of sorafenib. Additionally, the potentiation and the degree of additivity of the combination of aplidine with sorafenib at said doses are provided.

TABLE XXXXVIII

| | % Inhibition | | | Expected | Actual | Potenti- | Degree of |
|---|---|---|---|---|---|---|---|
| Day | G2 | G3 | G5 | Response | Response | ation | Response |
| 11 | −2.9 | 1.4 | −0.9 | −1.5 | 0.6 | No | — |
| 14 | 17.1 | −1.2 | 20.0 | 15.9 | 4.1 | Yes | Additive |
| 18 | 20.7 | 22.3 | 46.1 | 43.0 | 3.1 | Yes | Additive |
| 20 | 1.5 | 28.3 | 48.0 | 29.8 | 18.2 | Yes | Greater |
| | | | | | | | than |
| | | | | | | | additive |
| 22 | −2.0 | 35.2 | 42.1 | 33.2 | 8.9 | Yes | Greater |
| | | | | | | | than |
| | | | | | | | additive |
| 25 | −5.4 | 26.7 | 35.1 | 21.3 | 13.8 | Yes | Greater |
| | | | | | | | than |
| | | | | | | | additive |

Table XXXXIX shows the % of tumor growth inhibition of aplidine and sorafenib administered as single agents and in combination against LOX-IMVI human melanoma xenograft at a dose of 0.06 mg/kg/day of aplidine and 30 mg/kg/day of sorafenib. Additionally, the potentiation and the degree of additivity of the combination of aplidine with sorafenib at said doses are provided.

TABLE XXXXIX

| | % Inhibition | | | Expected | Actual | Potenti- | Degree of |
|---|---|---|---|---|---|---|---|
| Day | G2 | G4 | G6 | Response | Response | ation | Response |
| 11 | −2.9 | −2.1 | 2.7 | −5.0 | 7.7 | Yes | Additive |
| 14 | 17.1 | 3.1 | 23.3 | 20.2 | 3.1 | Yes | Additive |
| 18 | 20.7 | 22.6 | 48.5 | 43.3 | 5.2 | Yes | Additive |
| 20 | 1.5 | 24.1 | 48.7 | 25.6 | 23.1 | Yes | Greater |
| | | | | | | | than |
| | | | | | | | additive |
| 22 | −2.0 | 22.7 | 34.4 | 20.7 | 13.7 | Yes | Greater |
| | | | | | | | than |
| | | | | | | | additive |
| 25 | −5.4 | 6.6 | 17.3 | 1.2 | 16.1 | Yes | Greater |
| | | | | | | | than |
| | | | | | | | additive |

When aplidine and sorafenib were administered as single agents (monotherapy) against LOX-IMVI human melanoma cell line, they were inactive. Additionally, these treatments did not cause a significant decline in mean body weight, and all mice gained weight by the end of the study.

However, when aplidine, at a dose of 0.060 mg/kg/day, was combined with sorafenib at both doses of 30 and 60 mg/kg/day, a potentiation of activity was observed, resulting in a greater than additive potentiation of tumor growth inhibition. In addition, the combination of aplidine with sorafenib caused an acceptable decline in body weight, and all mice gained weight by the end of the study.

1. A method of treating cancer comprising administering to a patient in need of such treatment a therapeutically effective amount of aplidine, or a pharmaceutically acceptable salt thereof, and a therapeutically effective amount of another anticancer drug selected from sorafenib, temsirolimus, and sunitinib, or a pharmaceutically acceptable salt thereof.

2. A method of potentiating the therapeutic efficacy of an anticancer drug selected from sorafenib, temsirolimus, and sunitinib in the treatment of cancer, which comprises administering, to a patient in need thereof, said anticancer drug and a therapeutically effective amount of aplidine, or a pharmaceutically acceptable salt thereof.

3-15. (canceled)

16. The method according to claim 1, wherein the cancer to be treated is selected from renal carcinoma, hepatocarcinoma, melanoma, breast cancer, lung cancer, pancreatic cancer, neuroblastoma, and gastrointestinal stromal tumor (GIST).

17. The method according to claim 2, wherein the cancer to be treated is selected from renal carcinoma, hepatocarcinoma, melanoma, breast cancer, lung cancer, pancreatic cancer, neuroblastoma, and gastrointestinal stromal tumor (GIST).

18. The method according to claim 16, wherein aplidine, or a pharmaceutically acceptable salt thereof, and the other anticancer drag selected from sorafenib, temsirolimus, and sunitinib, or a pharmaceutically acceptable salt thereof, form part of the same composition.

19. The method according to claim 16, wherein aplidine, or a pharmaceutically acceptable salt thereof, and the other anticancer drug selected from sorafenib, temsirolimus, and sunitinib, or a pharmaceutically acceptable salt thereof, are provided as separate compositions for administration at the same time or at different times.

20. The method according to claim 19, wherein aplidine, or a pharmaceutically acceptable salt thereof, and the other anticancer drug selected from sorafenib, temsirolimus, and sunitinib, or a pharmaceutically acceptable salt thereof, are provided as separate compositions for administration at different times.

21. The method according to claim 20, wherein the anticancer drug combined with aplidine is sorafenib or a pharmaceutically acceptable salt thereof.

22. The method according to claim 20, wherein the anticancer drug combined with aplidine is temsirolimus or a pharmaceutically acceptable salt thereof.

23. The method according to claim 20, wherein the anticancer drug combined with aplidine is sunitinib or a pharmaceutically acceptable salt thereof.

24. The method according to claim 17, wherein aplidine, or a pharmaceutically acceptable salt thereof, and the other anticancer drug selected from sorafenib, temsirolimus, and sunitinib, or a pharmaceutically acceptable salt thereof, form part of the same composition.

25. The method according to claim 17, wherein aplidine, or a pharmaceutically acceptable salt thereof, and the other anticancer drug selected from sorafenib, temsirolimus, and sunitinib, or a pharmaceutically acceptable salt thereof, are provided as separate compositions for administration at the same time or at different times.

26. The method according to claim 25, wherein aplidine, or a pharmaceutically acceptable salt thereof, and the other anticancer drug selected from sorafenib, temsirolimus, and sunitinib, or a pharmaceutically acceptable salt thereof, are provided as separate compositions for administration at different times.

27. The method according to claim 26, wherein the anticancer drug combined with aplidine is sorafenib or a pharmaceutically acceptable salt thereof.

28. The method according to claim 26, wherein the anticancer drug combined with aplidine is temsirolimus or a pharmaceutically acceptable salt thereof.

29. The method according to claim 26, wherein the anticancer drug combined with aplidine is sunitinib or a pharmaceutically acceptable salt thereof.

30. A kit for administering aplidine, or a pharmaceutically acceptable salt thereof, in combination with another anticancer drug selected from sorafenib, temsirolimus, and sunitinib, or a pharmaceutically acceptable salt thereof in the treatment of cancer, comprising a dosage form of aplidine, or a pharmaceutically acceptable salt thereof, and/or a dosage form of the other anticancer drug selected from sorafenib, temsirolimus, and sunitinib, or a pharmaceutically acceptable salt thereof, and printed instructions for administering both drugs in combination.
